(19) United States
(12) Patent Application Publication
(10) Pub. No.: US 2010/0234409 A1
(43) Pub. Date: Sep. 16, 2010

(54) Amino cyclopentyl heterocyclic and carbocyclic modulators of chemokine receptor activity

(76) Inventors: Lihu Yang, Edison, NJ (US); Songnian Lin, Monroe, NJ (US); Gregori Morriello, Randolph, NJ (US); Liangqin Guo, Edison, NJ (US); Changyou Zhou, Plainsboro, NJ (US)

Correspondence Address: MERCK, P O BOX 2000, RAHWAY, NJ, 07065-0907, US

(21) Appl. No.: 11/596,937

(22) PCT Filed: May 20, 2005
(86) PCT No.: PCT/US05/17836

§ 371 Date: Nov. 17, 2006

Related U.S. Application Data
(60) Provisional application No. 60/573,625, filed on May 21, 2004.

(51) Int. Cl. A61K 31/438; C07D 401/10; C07D 211/32; C07D 235/04; C07D 215/04; A61K 31/445; A61K 31/454; A61K 31/4184; A61P 37/02; A61P 19/02

(52) U.S. Cl. 514/278; 546/17; 546/199; 548/304.7; 546/16; 514/322; 514/315; 514/394

(57) ABSTRACT

Compounds of the formulae (I) and (II): (wherein Q, X, E, G¹, G², R², R³, R⁴, R⁵, R⁶ and Z are as defined herein) which are modulators of chemokine receptor activity and are useful in the prevention or treatment of certain inflammatory and immunoregulatory disorders and diseases, allergic diseases, atopic conditions including allergic rhinitis, dermatitis, conjunctivitis, and asthma, as well as autoimmune pathologies such as rheumatoid arthritis and atherosclerosis. The invention is also directed to pharmaceutical compositions comprising these compounds and the use of these compounds and compositions in the prevention or treatment of such diseases in which chemokine receptors are involved.

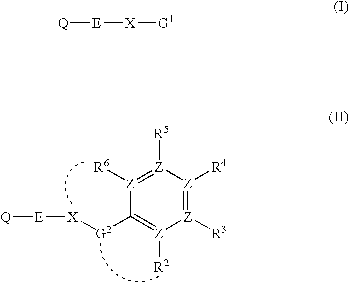

### BACKGROUND OF THE INVENTION

The chemokines are a family of small (70-120 amino acids), proinflammatory cytokines, with potent chemotactic activities. Chemokines are chemotactic cytokines that are released by a wide variety of cells to attract various cells, such as monocytes, macrophages, T cells, eosinophils, basophils and neutrophils to sites of inflammation (reviewed in Schall, Cytokine, 3, 165-183 (1991) and Murphy, Rev. Immun., 12, 593-633 (1994)). These molecules were originally defined by four conserved cysteines and divided into two subfamilies based on the arrangement of the first cysteine pair. In the CXC-chemokine family, which includes IL-8, GROα, NAP-2 and IP-10, these two cysteines are separated by a single amino acid, while in the CC-chemokine family, which includes RANTES, MCP-1, MCP-2, MCP-3, MT-1α, MIP-1β and eotaxin, these two residues are adjacent.

The α-chemokines, such as interleukin-8 (IL-8), neutrophil-activating protein-2 (NAP-2) and melanoma growth stimulatory activity protein (MGSA) are chemotactic primarily for neutrophils, whereas β-chemokines, such as RANTES, MIP-1α, MIP-1β, monocyte chemotactic protein-1 (MCP-1), MCP-2, MCP-3 and eotaxin are chemotactic for macrophages, monocytes, T-cells, eosinophils and basophils (Deng, et al., Nature, 381, 661-666 (1996)).

The chemokines are secreted by a wide variety of cell types and bind to specific G-protein coupled receptors (GPCRs) (reviewed in Horuk, Trends Pharm. Sci., 15, 159-165 (1994)) present on leukocytes and other cells. These chemokine receptors form a sub-family of GPCRs, which, at present, consists of fifteen characterized members and a number of orphans. Unlike receptors for promiscuous chemoattractants such as C5a, fMLP, PAF, and LTB4, chemokine receptors are more selectively expressed on subsets of leukocytes. Thus, generation of specific chemokines provides a mechanism for recruitment of particular leukocyte subsets.

On binding their cognate ligands, chemokine receptors transduce an intracellular signal though the associated trimeric G protein, resulting in a rapid increase in intracellular calcium concentration. There are at least seven human chemokine receptors that bind or respond to (3-chemokines with the following characteristic pattern: CCR-1 (or “CKR-1” or “CC-CKR-1”) [MIP-1α, MIP-1β, MCP-3, RANTES] (Ben-Barruch, et al., J. Biol. Chem., 270, 22123-22128 (1995); Beote, et al, Cell, 72, 415-425 (1993)); CCR-2A and CCR-2B (or “CKR-2A”/“CKR-2A” or “CC-CKR-2A”/“CC-CKR-2A”) [MCP-1, MCP-2, MCP-3, MCP-4]; CCR-3 (or “CKR-3” or “CC-CKR-3”) [Eotaxin, Eotaxin 2, RANTES, MCP-2, MCP-3] (Rollins, et al., Blood, 90, 908-928 (1997)); CCR-4 (or “CKR-4” or “CC-CKR-4”) [MIP-1α RANTES, MCP-1] (Rollins, et al., Blood, 90, 908-928 (1997)); CCR-5 (or “CKR-5” or “CC-CKR-5”) [MIP-1α RANTES, MIP-1β] (Sanson, et al., Biochemistry, 35, 3362-3367 (1996)); and the Duffy blood-group antigen [RANTES, MCP-1] (Chaudhun, et al., J. Biol. Chem., 269, 7835-7838 (1994)). The β-chemokines include eotaxin, MIP (“macrophage inflammatory protein”), MCP (“monocyte chemoattractant protein”) and RANTES (“regulation-upon-activation, normal T expressed and secreted”) among other chemokines.

Chemokine receptors, such as CCR-1, CCR-2, CCR-2A, CCR-2B, CCR-3, CCR-4, CCR-5, CXCR-3, CXCR-4, have been implicated as being important mediators of inflammatory and immunoregulatory disorders and diseases, including asthma, rhinitis and allergic diseases, as well as autoimmune pathologies such as rheumatoid arthritis and atherosclerosis. Humans who are homozygous for the 32-basepair deletion in the CCR-5 gene appear to have less susceptibility to rheumatoid arthritis (Gomez, et al., Arthritis & Rheumatism, 42, 989-992 (1999)). A review of the role of eosinophils in allergic inflammation is provided by Kita, H., et al., J. Exp. Med. 183, 2421-2426 (1996). A general review of the role of chemokines in allergic inflammation is provided by Lustger, A. D., New England J. Med., 338(7), 426-445 (1998).

A subset of chemokines are potent chemoattractants for monocytes and macrophages. The best characterized of these is MCP-1 (monocyte chemoattractant protein-1), whose primary receptor is CCR2. MCP-1 is produced in a variety of cell types in response to inflammatory stimuli in various species, including rodents and humans, and stimulates chemotaxis in monocytes and a subset of lymphocytes. In particular, MCP-1 production correlates with monocyte and macrophage infiltration at inflammatory sites. Deletion of either MCP-1 or CCR2 by homologous recombination in mice results in marked attenuation of monocyte recruitment in response to thioglycollate injection and Listeria monocytogenes infection (Lu et al., J. Exp. Med., 187, 601-608 (1998); Kurihara et al. J. Exp. Med., 186, 1757-1762 (1997); Boring et al. J. Clin. Invest., 100, 2552-2561 (1997); Kuziel et al. Proc. Natl. Acad. Sci., 94, 12053-12058 (1997)). Furthermore, these animals show reduced monocyte infiltration into granulomatous lesions induced by the injection of schistosomal or mycobacterial antigens (Boring et al. J. Clin. Invest., 100, 2552-2561 (1997); Warmington et al. Am J. Path., 154, 1407-1416 (1999)). These data suggest that MCP-1-induced CCR2 activation plays a major role in monocyte recruitment to inflammatory sites, and that antagonism of this activity will produce a sufficient suppression of the immune response to produce therapeutic benefits in immunoinflammatory and autoimmune diseases.

Accordingly, agents which modulate chemokine receptors such as the CCR-2 receptor would be useful in such disorders and diseases.

In addition, the recruitment of monocytes to inflammatory lesions in the vascular wall is a major component of the pathogenesis of atherogenic plaque formation. MCP-1 is produced and secreted by endothelial cells and intimal smooth muscle cells after injury to the vascular wall in hypercholesterolemic conditions. Monocytes recruited to the site of injury infiltrate the vascular wall and differentiate to foam cells in response to the released MCP-1. Several groups have now demonstrated that aortic lesion size, macrophage content and necrosis are attenuated in MCP-1−/− or CCR2−/− mice backcrossed to APO-E−/−, LDL-R−/− or Apo B transgenic mice maintained on high fat diets (Boring et al. Nature, 394, 894-897 (1998); Gosling et al. J. Clin. Invest., 103, 773-778 (1999)). Thus, CCR2 antagonists may inhibit atherosclerotic lesion formation and pathological progression by impairing monocyte recruitment and differentiation in the arterial wall.

### SUMMARY OF THE INVENTION

The present invention is directed to compounds of the formulae I and II:

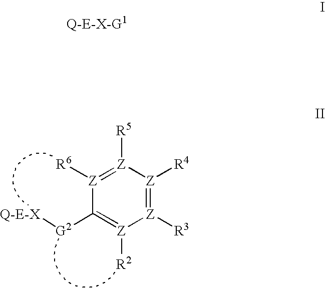

(wherein Q, X, E, G¹, G², R², R³, R⁴, R⁵, R⁶ and Z are as defined herein) which are modulators of chemokine receptor activity and are useful in the prevention or treatment of certain inflammatory and immunoregulatory disorders and diseases, allergic diseases, atopic conditions including allergic rhinitis, dermatitis, conjunctivitis, and asthma, as well as autoimmune pathologies such as rheumatoid arthritis and atherosclerosis. The invention is also directed to pharmaceutical compositions comprising these compounds and the use of these compounds and compositions in the prevention or treatment of such diseases in which chemokine receptors are involved.

### DETAILED DESCRIPTION OF THE INVENTION

The present invention is reacted to compounds of the formulae I and II:

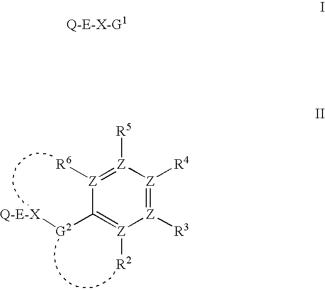

wherein:

### Q is:

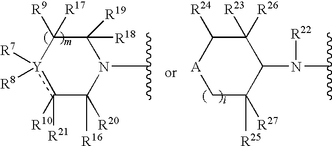

A is selected from: —O—, —NR¹²—, —S—, —SO—, —SO₂—, —CR¹²R¹²—, —NSO₂R¹⁴—, —NCOR¹³—, —CR¹²COR¹¹—, —CR¹²OCOR¹³— and —CO—;

### E is:

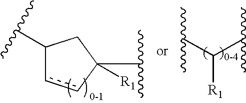

G¹ is selected from: —N(R³¹)—CO—N(R³⁰)(R²⁹), —N(R³¹)—SO₂R³², —N(R³¹)—COR³², —CON(R²⁹)(R³⁰), —C₁₋₆alkyl unsubstituted or substituted with 1-6 fluoro, and —C₃₋₆cycloalkyl unsubstituted or substituted with 1-6 fluoro,
where R²⁹ and R³⁰ are independently selected from: hydrogen, C₁₋₆alkyl, C₁₋₆alkyl substituted with 1-6 fluoro, C₁₋₆cycloalkyl, aryl, aryl-C₁₋₆alkyl, heterocycle and heterocycle-C₁₋₆alkyl, or R²⁹ and R³⁰ join to form a C₃₋₆ membered ring;
where R³¹ and R³² are independently selected from: hydrogen, C₁₋₆alkyl, C₁₋₆cycloalkyl, C₁₋₆alkyl substituted with 1-6 fluoro, aryl and heterocycle, or R³¹ and R³² join to form a C₃₋₆ membered ring;
   G² is selected from (where either end of the group is joined to X and the other end is joined to the aromatic ring): a single bond, —(CR¹¹R¹¹)₁₋₄—, —N(R¹²)SO₂—, —N(R¹²)SO₂N(R¹²)—, —N(R¹²)CO—, —C(R¹¹)(R¹¹)CO—, —C(R¹¹)(R¹¹)OCO—, —CO—, —C(R¹¹)(R¹¹)SO₂—, —OCO—, —SO₂—, or G² is CR¹¹ or N and is joined to R² forming a fused carbocyclic or heterocyclic ring;
   X is a 5-7 membered saturated, partially unsaturated or unsaturated carbocyclic or heterocyclic ring, wherein:
when said ring is heterocyclic it contains 1-4 heteroatoms independently selected from O, N and S,
said ring is unsubstituted or substituted with 1-4 R²⁸,
R²⁸ is independently selected from: halo, hydroxy, —O—C1-3alkyl unsubstituted or substituted with 1-6 fluoro, C1-3alkyl unsubstituted or substituted with 1-6 fluoro, —O—C3-5cycloalkyl unsubstituted or substituted with 1-6 fluoro, —COR11, —SO2R14, —NR¹²COR¹³, —NR¹²SO₂R¹⁴, -phenyl unsubstituted or substituted with 1-3 fluoro or trifluoromethyl, and —CN, and
said ring is optionally bonded to R⁶ to form a fused or spiro ring system (as shown by the curving dashed line in formula II);

### Y is C, N, O, S or SO₂;

Z is independently selected from C and N, where no more than two of Z are N;
R¹ is selected from: hydrogen, —SO₂R¹⁴, —C₀₋₃alkyl-S(O)R¹⁴, —SO₂NR¹²R¹², —C₁₋₆alkyl, —C₀₋₆alkyl-O—C₁₋₆alkyl, —C₀₋₆alkyl-S—C₁₋₆alkyl, —(C₀₋₆alkyl)-(C₃₋₇cycloalkyl)-(C₀₋₆alkyl), hydroxy, heterocycle, —CN, —NR¹²R¹², —NR¹²COR¹³, —NR¹²SO₂R¹⁴, —COR¹¹, —CONR¹²R¹², and phenyl,
wherein said alkyl and the cycloalkyl are unsubstituted or substituted with 1-7 substituents where the substituents are independently selected from: halo, hydroxy, —O—C₁₋₃alkyl, trifluoromethyl, C₁₋₃alkyl, —O—C₁₋₅cycloalkyl, —COR¹¹, —SO₂R¹⁴, —NHCOCH₃, —NHSO₂CH₃, -heterocycle, ═O and —CN, and
wherein said phenyl and heterocycle are unsubstituted or substituted with 1-3 substituents independently selected from halo, hydroxy, C₁₋₃alkyl, C₁₋₃alkoxy and trifluoromethyl;
   R³, R⁴, and R⁵ are independently selected from B¹ when Z is C, and are independently selected from B² when Z is N;
   R² is independently selected from B¹ when Z is C, and is independently selected from B² when Z is N, or R² is a link to G² wherein said link is a bond or is a chain 1-4 atoms in length where said atoms are independantly selected from O, N, C and S and where said atoms are independantly joined by single or double bonds, said link forming a fused carbocyclic or heterocyclic ring;
   R⁶ is independently selected from B¹ when Z is C, and is independently selected from B² when Z is N, or R⁶ is a link to any atom on X, wherein said link is a bond or is a chain 1-3 atoms in length where said atoms are independantly selected from O, N, C and S and where said atoms are independantly joined by single or double bonds, said link forming a fused carbocyclic or heterocyclic ring;
   B¹ is selected from: C₁₋₆alkyl unsubstituted or substituted with 1-6 fluoro, hydroxyl, or both, —O—C₁₋₆alkyl unsubstituted or substituted with 1-6 fluoro, —CO—C₁₋₆alkyl unsubstituted or substituted with 1-6 fluoro, —S—C₁₋₆alkyl unsubstituted or substituted with 1-6 fluoro, -pyridyl unsubstituted or substituted with one or more substituents selected from the group consisting of: halo, trifluoromethyl, C₁₋₄alkyl and COR¹¹, fluoro, chloro, bromo, —C₄₋₆cycloalkyl, —O—C₄₋₆cycloalkyl, phenyl unsubstituted or substituted with one or more substituents selected from halo, trifluoromethyl, C₁₋₄alkyl and COR¹¹, —O-phenyl unsubstituted or substituted with one or more substituents selected from halo, trifluoromethyl, C₁₋₄alkyl and COR¹¹, —C₃₋₆cycloalkyl unsubstituted or substituted with 1-6 fluoro, —O—C₃₋₆cycloalkyl unsubstituted or substituted with 1-6 fluoro, -heterocycle, —CN, —COR¹¹ and hydrogen;
   B² is absent or is O (to form an N-oxide);
   R⁷ is selected from: hydrogen, (C₀₋₆alkyl)-phenyl, (C₀₋₆alkyl)-heterocycle, (C₀₋₆alkyl)-C₃₋₇cycloalkyl, (C₀₋₆alkyl)-COR¹¹, (C₀₋₆alkyl)-(alkene)-COR¹¹, (C₀₋₆alkyl)-SO₃H, (C₀₋₆alkyl)-W-C₀₋₄alkyl, (C₀₋₆alkyl)-CONR¹²-pheny and (C₀₋₆alkyl)-CONR¹⁵—V—COR¹¹ when Y is N or C, or R⁷ is absent when Y is O, S or SO₂, where
V is C₁₋₆alkyl or phenyl,
W is a single bond, —O—, —S—, —SO—, —SO₂—, —CO—, —CO₂—, —CONR¹²— or —NR¹²—,
R¹⁵ is hydrogen or C₁₋₄alkyl, or R¹⁵ is joined via a 1-5 carbon chain linked to one of the carbons of V, forming a ring,
said C₀₋₆alkyl is unsubstituted or substituted with 1-5 substituents independently selected from halo, hydroxy, —C₀₋₆alkyl, —O—C₁₋₃alkyl, trifluoromethyl and —C₀₋₂alkyl-phenyl,
said phenyl, heterocycle, cycloalkyl and C₀₋₄alkyl are unsubstituted or substituted with 1-5 substituents independently selected from halo, trifluoromethyl, hydroxy, C₁₋₆alkyl, —O—C₁₋₃alkyl, —C₀₋₃—COR¹¹, CN, —NR¹²R¹², —CONR¹²R¹² and —C₀₋₃-heterocycle, or said phenyl or heterocycle may be fused to another heterocycle where said another heterocycle is unsubstituted or substituted with 1-2 substituents independently selected from hydroxy, halo, —COR¹¹, and —C₁₋₄alkyl, and
said alkene is unsubstituted or substituted with 1-3 substituents independently selected from halo, trifluoromethyl, C₁₋₃alkyl, phenyl and heterocycle;
   R⁸ is selected from hydrogen, hydroxy, C₁₋₆alkyl, C₁₋₆alkyl-hydroxy, —O—C₁₋₃alkyl, —COR¹¹, —CONR¹²R¹² and —CN when Y is N or C, or R⁸ is absent when Y is O, S, SO₂ or N or when a double bond joins the carbons to which R⁷ and R¹⁰ are attached;
   or R⁷ and R⁸ are joined to form a ring selected from: 1H-indene, 2,3-dihydro-1H-indene, 2,3-dihydro-benzofuran, 1,3-dihydro-isobenzofuran, 2,3-dihydro-benzothiofuran, 1,3-dihydro-isobenzothiofuran, 6H-cyclopenta[d]isoxazol-3-ol, cyclopentane and cyclohexane,
where said ring is unsubstituted or substituted with 1-5 substituents independently selected from: halo, trifluoromethyl, hydroxy, C₁₋₃alkyl, —O—C₁₋₃alkyl, —C₀₋₃—COR¹¹, —CN, —NR¹²R¹², —CONR¹²R¹² and —C₀₋₃-heterocycle;
   R⁹ and R¹⁰ are independently selected from: hydrogen, hydroxy, C₁₋₆alkyl, C₁₋₆alkyl-COR¹¹, C₁₋₆alkyl-hydroxy, —O—C₁₋₃alkyl, halo and ═O (connected to the ring via a double bond);
   or R⁷ and R⁹, or R⁸ and R¹⁰, together form a ring which is phenyl or heterocycle, wherein said ring is unsubstituted or substituted with 1-7 substituents independently selected from halo, trifluoromethyl, hydroxy, C₁₋₃alkyl, —O—C₁₋₃alkyl, —COR¹¹, —CN, —NR¹²R¹² and —CONR¹²R¹²;
   R¹¹ is independently selected from: hydroxy, hydrogen, C₁₋₆ alkyl, —O—C₁₋₆alkyl, benzyl, phenyl and C₃₋₆cycloalkyl, where said alkyl, phenyl, benzyl and cycloalkyl are unsubstituted or substituted with 1-3 substituents independently selected from halo, hydroxy, C₁₋₃alkyl, C₁₋₃alkoxy, —CO₂H, —CO₂—C₁₋₆ alkyl and trifluoromethyl;
   R¹² is selected from: hydrogen, C₁₋₆ alkyl, benzyl, phenyl and C₃₋₆ cycloalkyl, where said alkyl, phenyl, benzyl, and cycloalkyl are unsubstituted or substituted with 1-3 substituents independently selected from halo, hydroxy, C₁₋₃alkyl, C₁₋₃alkoxy, —CO₂H, —CO₂—C₁₋₆ alkyl, and trifluoromethyl;
   R¹³ is selected from: hydrogen, C₁₋₆ alkyl, —O—C₁₋₆alkyl, benzyl, phenyl and C₃₋₆cycloalkyl, where said alkyl, phenyl, benzyl and cycloalkyl are unsubstituted or substituted with 1-3 substituents independently selected from halo, hydroxy, C₁₋₃alkyl, C₁₋₃alkoxy, —CO₂H, —CO₂—C₁₋₆ alkyl and trifluoromethyl;
   R¹⁴ is selected from: hydroxy, C₁₋₆ alkyl, —O—C₁₋₆alkyl, benzyl, phenyl and C₃₋₆ cycloalkyl, where said alkyl, phenyl, benzyl, and cycloalkyl are unsubstituted or substituted with 1-3 substituents independently selected from halo, hydroxy, C₁₋₃alkyl, C₁₋₃alkoxy, —CO₂H, —CO₂—C₁₋₆ alkyl, and trifluoromethyl;
   R¹⁶ and R¹⁸ are independently selected from: hydroxy, C₁₋₆alkyl, C₁₋₆alkyl-COR¹¹, C₁₋₆alkyl-hydroxy, —O—C₁₋₃alkyl, halo and hydrogen, where said alkyl is unsubstituted or substituted with 1-6 substituents independantly chosen from fluoro and hydroxyl;
   or R¹⁶ and R¹⁸ together are —C₁₋₄alkyl-, —C₀₋₂alkyl-O—C₁₋₃alkyl- or —C₁₋₃alkyl-O—C₀₋₂alkyl-, forming a bridge, where said alkyl groups are unsubstituted or substituted with 1-2 substituents selected from oxy (where the oxygen is joined to the bridge via a double bond), fluoro, hydroxy, methoxy, methyl and trifluoromethyl;
   R¹⁷, R¹⁹, R²⁰ and R²¹ are independently selected from: hydrogen, hydroxy, C₁₋₆alkyl-COR¹¹, C₁₋₆alkyl-hydroxy, —O—C₁₋₃alkyl, trifluoromethyl and halo;
   R²² is hydrogen or C₁₋₆alkyl unsubstituted or substituted with 1-3 substituents independently selected from halo, hydroxy, —CO₂H, —CO₂C₁₋₆alkyl and —O—C₁₋₃alkyl;
   R²³ is selected from: C₁₋₆alkyl unsubstituted or substituted with 1-6 substituents selected from fluoro, C₁₋₃alkoxy, hydroxyl and —COR¹¹, fluoro, —O—C₁₋₃alkyl unsubstituted or substituted with 1-3 fluoro, C₃₋₆ cycloalkyl, —O—C₃₋₆cycloalkyl, hydroxy, —COR¹¹, —OCOR¹³, and ═O (where the oxygen is connected to the ring via a double bond), or R²² and R²³ together are C₂₋₄alkyl or C₀₋₂alkyl-O—C₁₋₃alkyl, forming a 5-7 membered ring;
   R²⁴ is selected from: hydrogen, C₁₋₆alkyl unsubstituted or substituted with 1-6 substituents selected from fluoro, C₁₋₃alkoxy, hydroxyl and —COR¹¹, COR¹¹, hydroxyl and —O—C₁₋₆alkyl unsubstituted or substituted with 1-6 substituents selected from fluoro, C₁₋₃alkoxy, hydroxyl and —COR¹¹, or R²³ and R²⁴ together are C₁₋₄alkyl or C₀₋₃alkyl-O—C₀₋₃alkyl, forming a 3-6 membered ring;
   R²⁵ is selected from: hydrogen, C₁₋₆alkyl unsubstituted or substituted with 1-6 fluoro, fluoro, —O—C₃₋₆cycloalkyl and —O—C₁₋₃alkyl unsubstituted or substituted with 1-6 fluoro,
   or R²³ and R²⁵ together are C₂₋₃alkyl, forming a 5-6 membered ring, where said alkyl is unsubstituted or substituted with 1-3 substituents independently selected from halo, hydroxy, —COR¹¹, C₁₋₃alkyl, and C₁₋₃alkoxy,
   or R²³ and R²⁵ together are C₁₋₂alkyl-O—C₁₋₂alkyl, forming a 6-8 membered ring, where said alkyls are unsubstituted or substituted with 1-3 substituents independently selected from halo, hydroxy, —COR¹¹, C₁₋₃alkyl and C₁₋₃alkoxy,
   or R²³ and R²⁵ together are —O—C₁₋₂alkyl-O—, forming a 6-7 membered ring, where said alkyl is unsubstituted or substituted with 1-3 substituents independently selected from halo, hydroxy, —COR¹¹, C₁₋₃alkyl and C₁₋₃alkoxy;
   R²⁶ is selected from: C₁₋₆alkyl unsubstituted or substituted with 1-6 substituents selected from fluoro, C₁₋₃alkoxy, hydroxyl and —COR¹¹, fluoro, —O—C₁₋₃alkyl unsubstituted or substituted with 1-3 fluoro, C₃₋₆ cycloalkyl, —O—C₃₋₆cycloalkyl, hydroxyl and —COR¹¹, or R²⁶ is absent if R²³ is connected to the Q ring via double bond (as in the case where R²³ is ═O),
   or R²⁶ and R²³ together form a bridgeselected from —C₂₋₅alkyl-, —O—C₂₋₅alkyl-, —O—C₂₋₅alkyl-O—, and —C₁₋₃alkyl-O—C₁₋₃alkyl-, where said alkyls are unsubstituted or substituted with 1-6 fluoro;
   R²⁷ is selected from: hydrogen, C₁₋₆alkyl unsubstituted or substituted with 1-6 fluoro, fluoro, —O—C₃₋₆cycloalkyl, and —O—C₁₋₃alkyl unsubstituted or substituted with 1-6 fluoro;
   m, i, and n are independently selected from 0, 1 and 2;
   the dashed line represents an optional bond;
   and pharmaceutically acceptable salts thereof and individual diastereomers thereof.

Embodiments of the invention include those of formula Ia

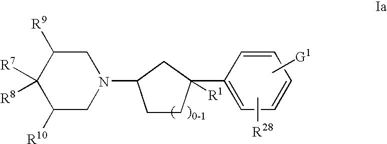

wherein R¹, R⁷, R⁸, R⁹, R¹⁰, R²⁸, and G¹ are defined herein, and pharmaceutically acceptable salts and individual diastereomers thereof.

Embodiments of the invention include those of formula Ib:

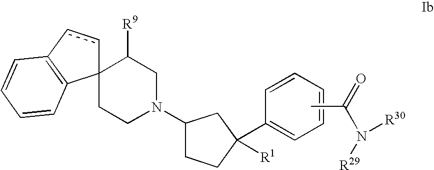

wherein R¹, R⁹, R²⁹, R³⁰, and the dashed line are defined herein, and pharmaceutically acceptable salts and individual diastereomers thereof.

Embodiments of the invention include those of formula Ic:

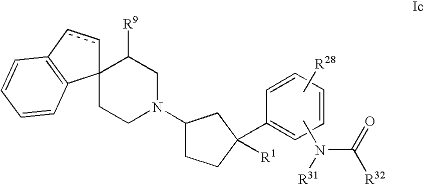

wherein R¹, R⁹, R²⁸, R³¹, R³² and the dashed line are defined herein, and pharmaceutically acceptable salts and individual diastereomers thereof.

Embodiments of the invention include those of formula Id:

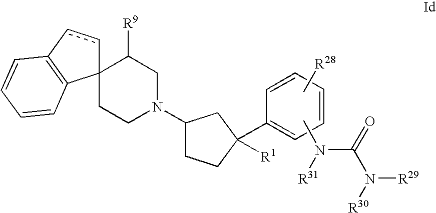

wherein R¹, R⁹, R²⁹, R³⁰, R³¹, R³², R²⁸ and the dashed line are defined herein, and pharmaceutically acceptable salts and individual diastereomers thereof.

Embodiments of the invention include those of formula Ie:

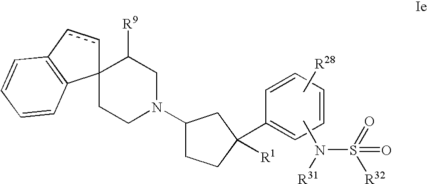

wherein R¹, R⁹, R³¹, R³², R²⁸ and the dashed line are defined herein, and pharmaceutically acceptable salts and individual diastereomers thereof.

Embodiments of the invention include those of formula IIa:

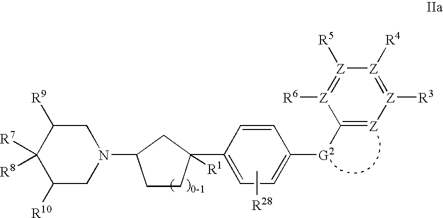

wherein R¹, R³, R⁴, R⁵, R⁶, R⁷, R⁸, R⁹, R¹⁰, R²⁸, G², and Z are defined herein, and pharmaceutically acceptable salts and individual diastereomers thereof.

Embodiments of the invention include those of formula IIb

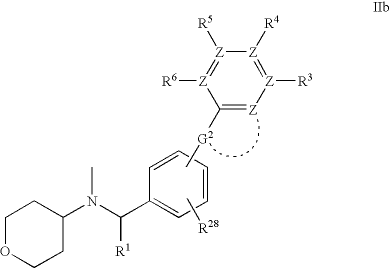

wherein R¹, R³, R⁴, R⁵, R⁶, R²⁸, and G² are defined herein.
and pharmaceutically acceptable salts and individual diastereomers thereof.

Embodiments of the invention include those of formula IIc:

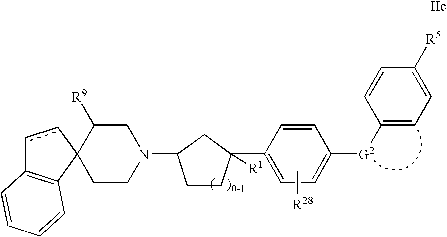

wherein R¹, R⁵, R⁹, R²⁸, G² and the dashed line are defined herein, and pharmaceutically acceptable salts and individual diastereomers thereof.

Embodiments of the invention include those of formula IId:

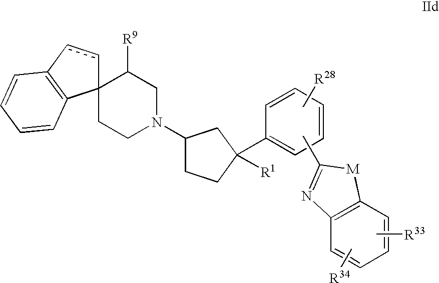

wherein
R¹, R⁹, R²⁸ and the dashed line are defined herein,
M is selected from O, S and NR¹²;
R³³ and R³⁴ are independently selected from hydrogen, halo, trifluoromethyl, O—C₁₋₆alkyl and O—C₁₋₆alkyl substituted with 1-6 fluoro, and pharmaceutically acceptable salts and individual diastereomers thereof.

Embodiments of the invention include those of formula IIe:

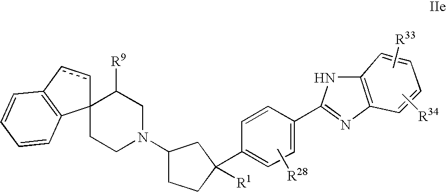

wherein R¹, R⁹, R³³, R³⁴, R²⁸ and the dashed line are defined herein, and pharmaceutically acceptable salts and individual diastereomers thereof.

Additional embodiments of the present invention include those wherein R²⁸ is selected from H, F, Cl, Br, Me and CF₃, and in particular those wherein R²⁸ is selected from H, Me and CF₃.

Additional embodiments of the present invention include wherein Y is C.

Additional embodiments of the present invention include those wherein A is O.

Additional embodiments of the present invention include those X is phenyl.

Additional embodiments of the present invention include those wherein R¹ is selected from hydrogen, —C₁₋₆alkyl unsubstituted or substituted with 1-6 substituents independently selected from halo, hydroxy, —O—C₁₋₃alkyl and trifluoromethyl, —C₀₋₆alkyl-O—C₁₋₆alkyl-unsubstituted or substituted with 1-6 substituents independently selected from halo and trifluoromethyl, unsubstituted or substituted with 1-6 substituents independently selected from halo and trifluoromethyl, —(C₃₋₅cycloalkyl)-(C₀₋₆alkyl) unsubstituted or substituted with 1-7 substituents independently selected from halo, hydroxy, —O—C₁₋₃alkyl and trifluoromethyl. In particular, embodiments incluse thoses wherein R¹ is selected from hydrogen, C₁₋₆alkyl, C₁₋₆alkyl-hydroxy and C₁₋₆alkyl substituted with 1-6 fluoro, specifically wherein R¹ is selected from hydrogen, methyl, hydroxymethyl and trifluoromethyl.

Additional embodiments of the present invention include those wherein when Z is N, R² is absent, and those wherein when Z is C, R² is hydrogen or is linked to G² as described herein.

Further embodiments of the present invention include those wherein if Z is N, R³ is absent, and those wherein if Z is C, R³ is hydrogen.

Further embodiments of the present invention include those wherein if the Z bonded to R⁴ is N, R⁴ is absent.

Further embodiments of the present invention include those wherein if the Z bonded to R⁴ is C, R⁴ is hydrogen.

Further embodiments of the present invention include those wherein, if the Z bonded to R⁵ is N, R⁵ is absent.

Further embodiments of the present invention include those wherein if the Z bonded to R⁶ is N, R⁶ is absent.

Further embodiments of the present invention include those wherein if the Z bonded to R⁶ is C, R⁶ is hydrogen.

Further embodiments of the present invention include those wherein R⁷ is selected from phenyl, heterocycle, C₃₋₇cycloalkyl, C₁₋₆alkyl, —COR¹¹ and —CONH—V—COR¹¹, where V is C₁₋₆alkyl or phenyl, and where said phenyl, heterocycle, C₃₋₇cycloalkyl and C₁₋₆alkyl is unsubstituted or substituted with 1-5 substituents independently selected from: halo, trifluoromethyl, hydroxy, —O—C₁₋₃alkyl, —COR¹¹, —CN, -heterocycle and —CONR¹²R¹².

Further embodiments of the present invention include those wherein R⁸ is selected from: hydrogen, hydroxy, —CN and —F.

Further embodiments of the present invention include those wherein R⁷ and R⁸ are joined together to form a ring which is selected from: 1H-indene and 2,3-dihydro-1H-indene, where said ring is unsubstituted or substituted with 1-3 substituents independently selected from: halo, hydroxy, C₁₋₃alkyl, —COR¹¹ and -heterocycle.

Further embodiments of the present invention include those wherein R⁹ and R¹⁰ are independently selected from: hydrogen, hydroxy, —CH₃, —O—CH₃ and ═O (where R⁹ and/or R¹⁰ are joined to the ring via a double bond).

Further embodiments of the present invention include those wherein R⁹ is hydrogen or methyl.

Further embodiments of the present invention include those wherein one or more of R¹⁰, R¹⁶, R¹⁷, R¹⁸, R¹⁹, R²⁰, R²¹, R²³, R²⁴, R²⁵, R²⁶ and R²⁷ is hydrogen.

Further embodiments of the present invention include those wherein R²² is methyl.

Representative compounds of the present invention include those described in the Examples, below, and pharmaceutically acceptable salts and individual diastereomers thereof.

The compounds of the instant invention where E is the cyclopentyl ring have at least two asymmetric centers at the 1- and 3-positions of the cycloalkyl ring. Additional asymmetric centers may be present depending upon the nature of the various substituents on the molecule. Each such asymmetric center will independently produce two optical isomers and, it is intended that all of the possible optical isomers and diastereomers in mixtures and as pure or partially purified compounds are included within the ambit of this invention. The absolute configurations of the more preferred compounds of this orientation where the substituents on the cycloalkyl ring (amide and amine units) are cis, as depicted:

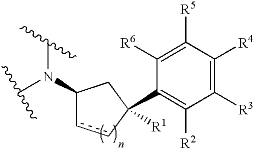

The absolute configurations of the most preferred compounds of this invention are those of the orientation as depicted:

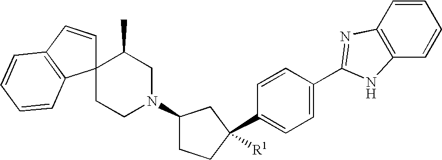

wherein the carbon bearing the amine substituent is designated as being of the (R) absolute configuration and the carbon bearing the amide subunit can be designated as being of either the (S) or (R) absolute configuration depending on the priority for R¹. For example if R is isopropyl then the absolute stereochemistry at the carbon bearing the amide subunit would be (S) since the amide and amine units are preferred to have the cis arrangement on the cyclopentyl ring.

The independent syntheses of diastereomers and enantiomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

As appreciated by those of skill in the art, halo or halogen as used herein are intended to include chloro, fluoro, bromo and iodo.

As used herein, “alkyl” is intended to mean linear, branched and cyclic carbon structures having no double or triple bonds. C₁₋₈, as in C₁₋₈alkyl, is defined to identify the group as having 1, 2, 3, 4, 5, 6, 7 or 8 carbons in a linear or branched arrangement, such that C₁₋₈alkyl specifically includes methyl, ethyl, n-propyl, iso-propyl, n-butyl, iso-butyl, tert-butyl, pentyl, hexyl, heptyl and octyl. More broadly, C_{a-b}alkyl (where a and b represent whole numbers) is defined to identify the group as having a through b carbons in a linear or branched arrangement. C₀, as in C₀alkyl is defined to identify the presence of a direct covalent bond. “Cycloalkyl” is an alkyl, part or all of which forms a ring of three or more atoms.

The term “heterocycle” as used herein is intended to include the following groups: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof.

The term “ring” is employed herein to refer to the formation or existence of a cyclic structure of any type, including free standing rings, fused rings, and bridges formed on existing rings. Rings may be non-aromatic or aromatic. Moreover, the existence or formation of a ring structure is at times herein disclosed wherein multiple substituents are defined “together”, as in “ . . . R⁸ and R⁹ together are C₁₋₄alkyl . . . ”. In this case a ring is necessarily formed regardless of whether the term “ring” is employed.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention can be prepared from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media such as ether, ethyl acetate, ethanol, isopropanol, or acetonitrile are employed. Suitable salts are found, e.g. in Remington's Pharmaceutical Sciences, 17th ed., Mack Publishing Company, Easton, Pa., 1985, p. 1418.

Specific compounds within the present invention include a compound which selected from the group consisting of those compounds described in the Examples, and pharmaceutically acceptable salts thereof and individual diastereomers and enantiomers thereof.

The subject compounds are useful in a method of modulating chemokine receptor activity in a patient in need of such modulation comprising the administration of an effective amount of the compound.

The present invention is directed to the use of the foregoing compounds as modulators of chemokine receptor activity. In particular, these compounds are useful as modulators of the chemokine receptors, in particular CCR-2.

The utility of the compounds in accordance with the present invention as modulators of chemokine receptor activity may be demonstrated by methodology known in the art, such as the assay for chemokine binding as disclosed by Van Riper, et al., J. Exp. Med., 177, 851-856 (1993) which may be readily adapted for measurement of CCR-2 binding.

Receptor affinity in a CCR-2 binding assay was determined by measuring inhibition of ¹²⁵I-MCP-1 to the endogenous CCR-2 receptor on various cell types including monocytes, THP-1 cells, or after heterologous expression of the cloned receptor in eukaryotic cells. The cells were suspended in binding buffer (50 mM HEPES, pH 7.2, 5 mM MgCl₂, 1 mM CaCl₂, and 0.50% BSA or 0.5% human serum) and added to test compound or DMSO and ¹²⁵I-MCP-1 at room temperature for 1 h to allow binding. The cells were then collected on GFB filters, washed with 25 mM HEPES buffer containing 500 mM NaCl and cell bound ¹²⁵I-MCP-1 was quantified.

In a chemotaxis assay chemotaxis was performed using T cell depleted PBMC isolated from venous whole or leukophoresed blood and purified by Ficoll-Hypaque centrifugation followed by rosetting with neuraminidase-treated sheep erythrocytes. Once isolated, the cells were washed with HBSS containing 0.1 mg/ml BSA and suspended at 1×10⁷ cells/ml. Cells were fluorescently labeled in the dark with 2 μM Calcien-AM (Molecular Probes), for 30 min at 37° C. Labeled cells were washed twice and suspended at 5×10⁶ cells/ml in RPMI 1640 with L-glutamine (without phenol red) containing 0.1 mg/ml BSA. MCP-1 (Peprotech) at 10 ng/ml diluted in same medium or medium alone were added to the bottom wells (27 μl). Monocytes (150,000 cells) were added to the topside of the filter (30 μl) following a 15 min preincubation with DMSO or with various concentrations of test compound. An equal concentration of test compound or DMSO was added to the bottom well to prevent dilution by diffusion. Following a 60 min incubation at 37° C., 5% CO₂, the filter was removed and the topside was washed with HBSS containing 0.1 mg/ml BSA to remove cells that had not migrated into the filter. Spontaneous migration (chemokinesis) was determined in the absence of chemoattractant.

In particular, the compounds of the following examples had activity in binding to the CCR-2 receptor in the aforementioned assays, generally with an IC₅₀ of less than about 1 μM. Such a result is indicative of the intrinsic activity of the compounds in use as modulators of chemokine receptor activity.

Mammalian chemokine receptors provide a target for interfering with or promoting eosinophil and/or leukocyte function in a mammal, such as a human. Compounds which inhibit or promote chemokine receptor function, are particularly useful for modulating eosinophil and/or leukocyte function for therapeutic purposes. Accordingly, compounds which inhibit or promote chemokine receptor function would be useful in treating, preventing, ameliorating, controlling or reducing the risk of a wide variety of inflammatory and immunoregulatory disorders and diseases, allergic diseases, atopic conditions including allergic rhinitis, dermatitis, conjunctivitis, and asthma, as well as autoimmune pathologies such as rheumatoid arthritis and atherosclerosis, and further, chronic obstructive pulmonary disease, and multiple schlerosis.

For example, an instant compound which inhibits one or more functions of a mammalian chemokine receptor (e.g., a human chemokine receptor) may be administered to inhibit (i.e., reduce or prevent) inflammation. As a result, one or more inflammatory processes, such as leukocyte emigration, chemotaxis, exocytosis (e.g., of enzymes, histamine) or inflammatory mediator release, is inhibited.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent or murine species can be treated. However, the method can also be practiced in other species, such as avian species (e.g., chickens).

Diseases and conditions associated with inflammation and infection can be treated using the compounds of the present invention. In a certain embodiment, the disease or condition is one in which the actions of leukocytes are to be inhibited or promoted, in order to modulate the inflammatory response.

Diseases or conditions of humans or other species which can be treated with inhibitors of chemokine receptor function, include, but are not limited to: inflammatory or allergic diseases and conditions, including respiratory allergic diseases such as asthma, particularly bronchial asthma, allergic rhinitis, hypersensitivity lung diseases, hypersensitivity pneumonitis, eosinophilic pneumonias (e.g., Loeffler's syndrome, chronic eosinophilic pneumonia), delayed-type hypersentitivity, interstitial lung diseases (ILD) (e.g., idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis); systemic anaphylaxis or hypersensitivity responses, drug allergies (e.g., to penicillin, cephalosporins), insect sting allergies; autoimmune diseases, such as rheumatoid arthritis, psoriatic arthritis, multiple sclerosis, systemic lupus erythematosus, myasthenia gravis, juvenile onset diabetes; glomerulonephritis, autoimmune thyroiditis, Behcet's disease; graft rejection (e.g., in transplantation), including allograft rejection or graft-versus-host disease; inflammatory bowel diseases, such as Crohn's disease and ulcerative colitis; spondyloarthropathies; scleroderma; psoriasis (including T-cell mediated psoriasis) and inflammatory dermatoses such an dermatitis, eczema, atopic dermatitis, allergic contact dermatitis, urticaria; vasculitis (e.g., necrotizing, cutaneous, and hypersensitivity vasculitis); eosinphilic myositis, eosinophilic fasciitis; and cancers, including cancers with leukocyte infiltration of the skin or organs and other cancers. Inhibitors of chemokine receptor function may also be useful in the treatment and prevention of stroke (Hughes et al., Journal of Cerebral Blood Flow & Metabolism, 22:308-317, 2002, and Takami et al., Journal of Cerebral Blood Flow & Metabolism, 22:780-784, 2002), neurodegenerative conditions including but not limited to Alzheimer's disease, amyotrophic lateral sclerosis (ALS) and Parkinson's disease, obesity, type II diabetes, neuropathic and inflammatory pain, and Guillain Barre syndrome. Other diseases or conditions in which undesirable inflammatory responses are to be inhibited can be treated, including, but not limited to, reperfusion injury, atherosclerosis, certain hematologic malignancies, cytokine-induced toxicity (e.g., septic shock, endotoxic shock), polymyositis, dermatomyositis and chronic obstructive pulmonary disease.

Diseases or conditions of humans or other species, which can be treated with modulators of chemokine receptor function, include or involve but are not limited to: immunosuppression, such as that in individuals with immunodeficiency syndromes such as AIDS or other viral infections, individuals undergoing radiation therapy, chemotherapy, therapy for autoimmune disease or drug therapy (e.g., corticosteroid therapy), which causes immunosuppression; immunosuppression due to congenital deficiency in receptor function or other causes; and infections diseases, such as parasitic diseases, including, but not limited to helminth infections, such as nematodes (round worms), (Trichuriasis, Enterobiasis, Ascariasis, Hookworm, Strongyloidiasis, Trichinosis, filariasis), trematodes (flukes) (Schistosomiasis, Clonorchiasis), cestodes (tape worms) (Echinococcosis, Taeniasis saginata, Cysticercosis), visceral worms, visceral larva migraines (e.g., Toxocara), eosinophilic gastroenteritis (e.g., Anisaki sp., Phocanema sp.), and cutaneous larva migraines (Ancylostona braziliense, Ancylostoma caninum).

In addition, treatment of the aforementioned inflammatory, allergic, infectious and autoimmune diseases can also be contemplated for agonists of chemokine receptor function if one contemplates the delivery of sufficient compound to cause the loss of receptor expression on cells through the induction of chemokine receptor internalization or delivery of compound in a manner that results in the misdirection of the migration of cells.

The compounds of the present invention are accordingly useful in treating, preventing, ameliorating, controlling or reducing the risk of a wide variety of inflammatory and immunoregulatory disorders and diseases, allergic conditions, atopic conditions, as well as autoimmune pathologies. In a specific embodiment, the present invention is directed to the use of the subject compounds for treating, preventing, ameliorating, controlling or reducing the risk of autoimmune diseases, such as rheumatoid arthritis, psoriatic arthritis and multiple schlerosis.

In another aspect, the instant invention may be used to evaluate putative specific agonists or antagonists of chemokine receptors, including CCR-2. Accordingly, the present invention is directed to the use of these compounds in the preparation and execution of screening assays for compounds that modulate the activity of chemokine receptors. For example, the compounds of this invention are useful for isolating receptor mutants, which are excellent screening tools for more potent compounds. Furthermore, the compounds of this invention are useful in establishing or determining the binding site of other compounds to chemokine receptors, e.g., by competitive inhibition. The compounds of the instant invention are also useful for the evaluation of putative specific modulators of the chemokine receptors, including CCR-2. As appreciated in the art, thorough evaluation of specific agonists and antagonists of the above chemokine receptors has been hampered by the lack of availability of non-peptidyl (metabolically resistant) compounds with high binding affinity for these receptors. Thus the compounds of this invention are commercial products to be sold for these purposes.

The present invention is further directed to a method for the manufacture of a medicament for modulating chemokine receptor activity in humans and animals comprising combining a compound of the present invention with a pharmaceutical carrier or diluent.

The present invention is further directed to the use of the present compounds in treating, preventing, ameliorating, controlling or reducing the risk of infection by a retrovirus, in particular, herpes virus or the human immunodeficiency virus (HIV) and the treatment of, and delaying of the onset of consequent pathological conditions such as AIDS. Treating AIDS or preventing or treating infection by HIV is defined as including, but not limited to, treating a wide range of states of HIV infection: AIDS, ARC (AIDS related complex), both symptomatic and asymptomatic, and actual or potential exposure to HIV. For example, the compounds of this invention are useful in treating infection by HIV after suspected past exposure to HIV by, e.g., blood transfusion, organ transplant, exchange of body fluids, bites, accidental needle stick, or exposure to patient blood during surgery.

In a further aspect of the present invention, a subject compound may be used in a method of inhibiting the binding of a chemokine to a chemokine receptor, such as CCR-2, of a target cell, which comprises contacting the target cell with an amount of the compound which is effective at inhibiting the binding of the chemokine to the chemokine receptor.

The subject treated in the methods above is a mammal, for instance a human being, male or female, in whom modulation of chemokine receptor activity is desired. “Modulation” as used herein is intended to encompass antagonism, agonism, partial antagonism, inverse agonism and/or partial agonism. In an aspect of the present invention, modulation refers to antagonism of chemokine receptor activity. The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention to the individual in need of treatment.

As used herein, the term “treatment” refers both to the treatment and to the prevention or prophylactic therapy of the aforementioned conditions.

Combined therapy to modulate chemokine receptor activity for thereby treating, preventing, ameliorating, controlling or reducing the risk of inflammatory and immunoregulatory disorders and diseases, including asthma and allergic diseases, as well as autoimmune pathologies such as rheumatoid arthritis and multiple sclerosis, and those pathologies noted above is illustrated by the combination of the compounds of this invention and other compounds which are known for such utilities.

For example, in treating, preventing, ameliorating, controlling or reducing the risk of inflammation, the present compounds may be used in conjunction with an antiinflammatory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal antiinflammatory agent, or a cytokine-suppressing antiinflammatory agent, for example with a compound such as acetaminophen, aspirin, codeine, biological TNF sequestrants, fentanyl, ibuprofen, indomethacin, ketorolac, morphine, naproxen, phenacetin, piroxicam, a steroidal analgesic, sufentanyl, sunlindac, tenidap, and the like. Similarly, the instant compounds may be administered with a pain reliever; a potentiator such as caffeine, an H2-antagonist, simethicone, aluminum or magnesium hydroxide; a decongestant such as phenylephrine, phenylpropanolamine, pseudophedrine, oxymetazoline, ephinephrine, naphazoline, xylometazoline, propylhexedrine, or levo-desoxy-ephedrine; an antiitussive such as codeine, hydrocodone, caramiphen, carbetapentane, or dextramethorphan; a diuretic; and a sedating or non-sedating antihistamine.

Likewise, compounds of the present invention may be used in combination with other drugs that are used in the treatment/prevention/suppression or amelioration of the diseases or conditions for which compounds of the present invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention may be used. Accordingly, the pharmaceutical compositions of the present invention include those that also contain one or more other active ingredients, in addition to a compound of the present invention.

Examples of other active ingredients that may be combined with CCR2 antagonists, such as the CCR2 antagonists compounds of the present invention, either administered separately or in the same pharmaceutical compositions, include, but are not limited to: (a) VLA-4 antagonists such as those described in U.S. Pat. No. 5,510,332, WO95/15973, WO96/01644, WO96/06108, WO96/20216, WO96/22966, WO96/31206, WO96/40781, WO97/03094, WO97/02289, WO 98/42656, WO98/53814, WO98/53817, WO98/53818, WO98/54207, and WO98/58902; (b) steroids such as beclomethasone, methylprednisolone, betamethasone, prednisone, dexamethasone, and hydrocortisone; (c) immunosuppressants such as cyclosporin, tacrolimus, rapamycin, EDG receptor agonists including FTY-720, and other FK-506 type immunosuppressants; (d) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, desloratadine, cetirizine, fexofenadine, descarboethoxyloratadine, and the like; (e) non-steroidal anti-asthmatics such as β2-agonists (terbutaline, metaproterenol, fenoterol, isoetharine, albuterol, bitolterol, and pirbuterol), theophylline, cromolyn sodium, atropine, ipratropium bromide, leukotriene antagonists (zafirlukast, montelukast, pranlukast, iralukast, pobilukast, SKB-106,203), leukotriene biosynthesis inhibitors (zileuton, BAY-1005); (f) non-steroidal antiinflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); (g) cyclooxygenase-2 (COX-2) inhibitors; (h) inhibitors of phosphodiesterase type IV (PDE-IV); (i) other antagonists of the chemokine receptors, especially CCR-1, CCR-2, CCR-3, CXCR-3, CXCR-4 and CCR-5; (j) cholesterol lowering agents such as HMG-CoA reductase inhibitors (lovastatin, simvastatin and pravastatin, fluvastatin, atorvastatin, rosuvastatin, and other statins), sequestrants (cholestyramine and colestipol), cholesterol absorption inhibitors (ezetimibe), nicotinic acid, fenofibric acid derivatives (gemfibrozil, clofibrat, fenofibrate and benzafibrate), and probucol; (k) anti-diabetic agents such as insulin, sulfonylureas, biguanides (metformin), α-glucosidase inhibitors (acarbose) and glitazones (troglitazone and pioglitazone); (l) preparations of interferon beta (interferon beta-1α, interferon beta-1β); (m) preparations of glatiramer acetate; (n) preparations of CTLA4Ig; (o) preparations of hydroxychloroquine, (p) Copaxone® and (q) other compounds such as 5-aminosalicylic acid and prodrugs thereof, antimetabolites such as azathioprine, 6-mercaptopurine and methotrexate, leflunomide, teriflunomide, and cytotoxic and other cancer chemotherapeutic agents.

The weight ratio of the compound of the present invention to the second active ingredient may be varied and will depend upon the effective dose of each ingredient. Generally, an effective dose of each will be used. Thus, for example, when a compound of the present invention is combined with an NSAID the weight ratio of the compound of the present invention to the NSAID will generally range from about 1000:1 to about 1:1000, or from about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles.)

The pharmaceutical composition and method of the present invention may further comprise other therapeutically active compounds as noted herein which are usually applied in the treatment of the above mentioned pathological conditions.

In treating, preventing, ameliorating, controlling or reducing the risk of conditions which require chemokine receptor modulation an appropriate dosage level will generally be about 0.0001 to 500 mg per kg patient body weight per day which can be administered in single or multiple doses. In certain embodiments the dosage level will be about 0.0005 to about 400 mg/kg per day; or from about 0.005 to about 300 mg/kg per day; or from about 0.01 to about 250 mg/kg per day, or from about 0.05 to about 100 mg/kg per day, or from about 0.5 to about 50 mg/kg per day. Within this range the dosage may be 0.0001 to 0.005, 0.005 to 0.05, 0.05 to 0.5, 0.5 to 5 or 5 to 50 mg/kg per day. For oral administration, the compositions may be provided in the form of tablets containing 0.01 to 1000 milligrams of the active ingredient, or 0.1 to 500, 1.0 to 400, or 2.0 to 300, or 3.0 to 200, particularly 0.01, 0.05, 0.1, 1, 4, 5, 10, 15, 20, 25, 30, 50, 75, 100, 125, 150, 175, 200, 250, 300, 400, 500, 600, 750, 800, 900, and 1000 milligrams of the active ingredient for the symptomatic adjustment of the dosage to the patient to be treated. The compounds may be administered on a regimen of 1 to 4 times per day, or once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Several methods for preparing the compounds of this invention are illustrated in the following Schemes and Examples. Starting materials are made by known procedures or as illustrated.

One of the principal routes used for preparation of compounds within the scope of the instant invention which bear a 1,1,3-trisubstituted cyclopentane framework is depicted in Scheme 1.

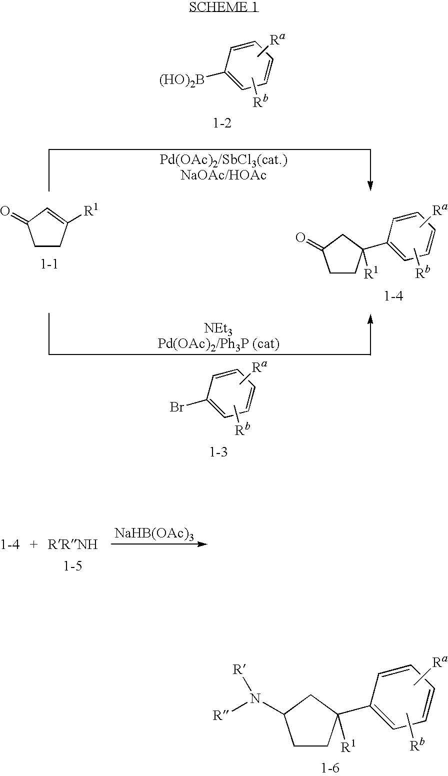

According to this route, 3-oxocyclopentylbenzene (1-4) which is synthesized by treatment of cyclopentenone (1-1) with functionalized benzene boronic acid (1-2) in the catalysis of palladium acetate and antimony chloride or with substituted bromobenzene (1-3) in the catalysis of palladium acetate/triphenyl phosphine in neat triethyl amine (Ref. H. A. Dieck & R. F. Heck, J. Am. Chem. Soc. 1974, 99, 1133). The resulting ketone then undergoes reductive amination in a presence of a reducing agent such as sodium triacetoxyborohydride or sodium cyanoborohydride to give the aminocyclopentyl benzene (1-6) which can be further converted into other chemokine modulators according to the procedures depicted in the following schemes.

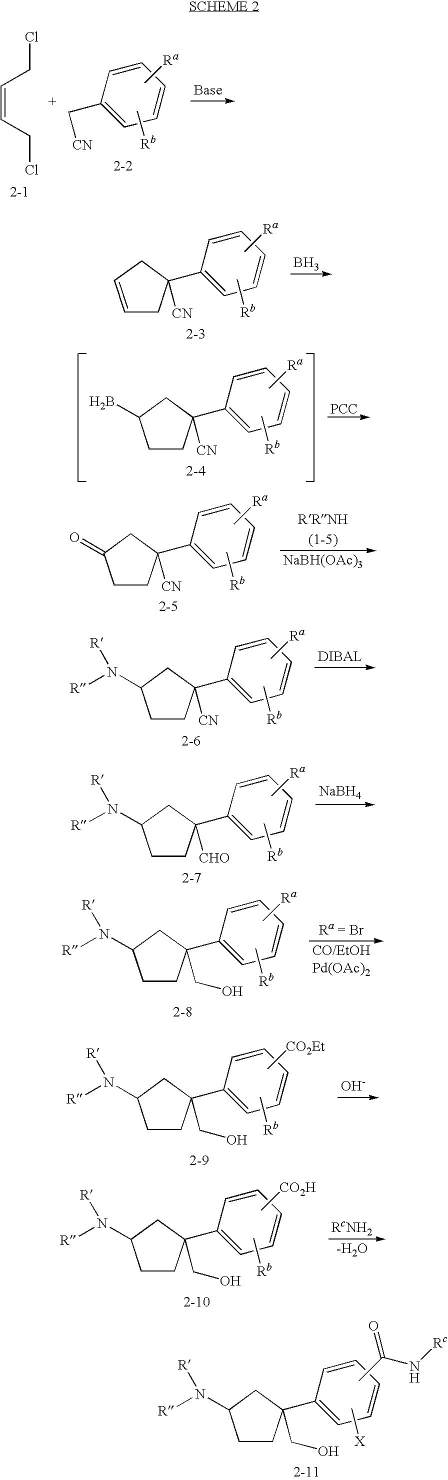

The cyclopentane core structures can also be prepared according to the Scheme 2. Treating a mixture of cis-1,4-dichloorobut-2-ene (2-1) and the substituted phenyl acetonitrile (2-2) in a mixture of DMF/DMPU gives the cyclopentene (2-3). Sequential reduction of the double bond with borane-THF and oxidation of the resulting intermediate (2-4) with PCC in one pot affords the corresponding cyclopentanone (2-5). The following reductive alkylation gives the aminocyclopentylbenzene (2-6) whose cyano group is further converted into the aldehyde (2-7). The alcohol (2-8) is prepared from the aldehyde (2-7) by reduction with sodium borihydride. The bromo atom in (2-8) can be converted into the ester (2-9) by heating it with palladium acetate in the atmosphere of carbon monoxide. Standard saponification of (2-9) and the following coupling of the resulting amino acid (2-10) with the amine afford the final chemokine modulator (2-11).

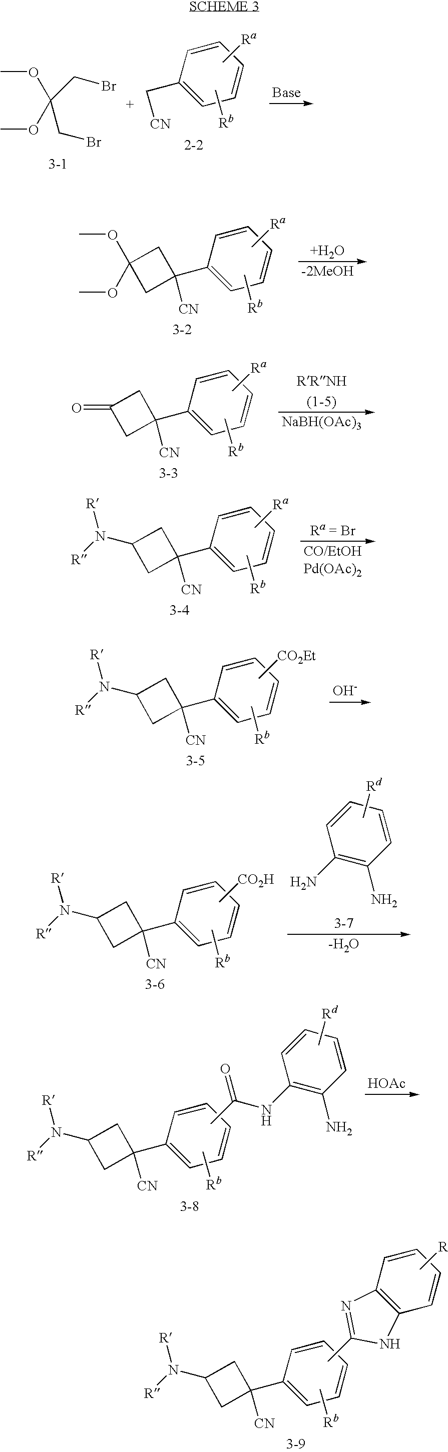

To prepare cyclobutane modulator, the double alkylation of substituted acetonitrile (2-2) with dimethyl-1,3-dibromo-acetal (3-1) is carried out using sodium hydride as a base. The following acidic hydrolysis of the resulting ketal (3-2) gives the corresponding cyclobutanone (3-3). After reduction amination, the aminocyclobutylbenzene (3-4) is obtained. Further derivatization of the bromobenzene (3-4) via a palladium chemistry results in the formation of the ester (3-5). After hydrolysis, EDC-initiated coupling of the acid (3-7) and acetic acid treatment of the coupling intermediate (3-8), the final imidazole modulator (3-9) is synthesized.

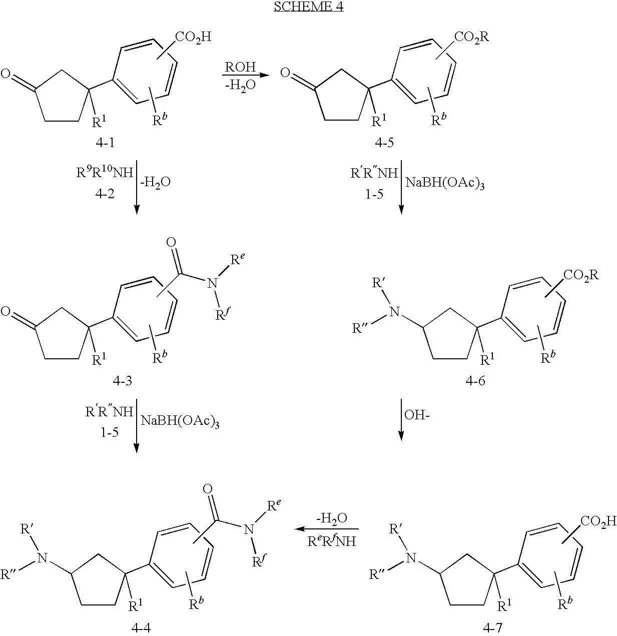

Two alternative routes can be used to prepare aminocyclopentyl benzene chemokine modulator. In the first route, the keto acid (4-1) is converted into the ketoamide (4-3) by EDC-initiated coupling with the amine (4-2). The following reductive amination affords the modulator (4-4). The second route starts from the keto ester which can be prepared by either procedure in Scheme 1 or by esterifying the keto acid. The resulted keto ester (4-5) is then converted into the amino ester (4-6). After hydrolysis, the amino acid (4-7) is obtained. The standard EDC-initiated coupling gives the final modulator (4-4).

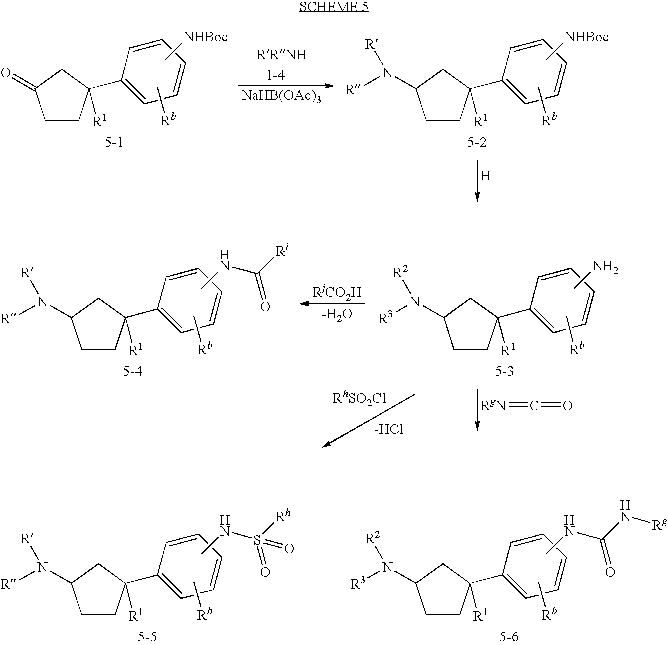

The carbamate (5-1), which is prepared according to the Scheme 1, undergoes reductive amination to give the amine (5-2). After removal of the protecting group, the aminocyclopentylaniline (5-3) is obtained. The (5-3) can be converted into various derivatives such as modulators (5-4), (5-5) and (5-6) based on standard chemistry.

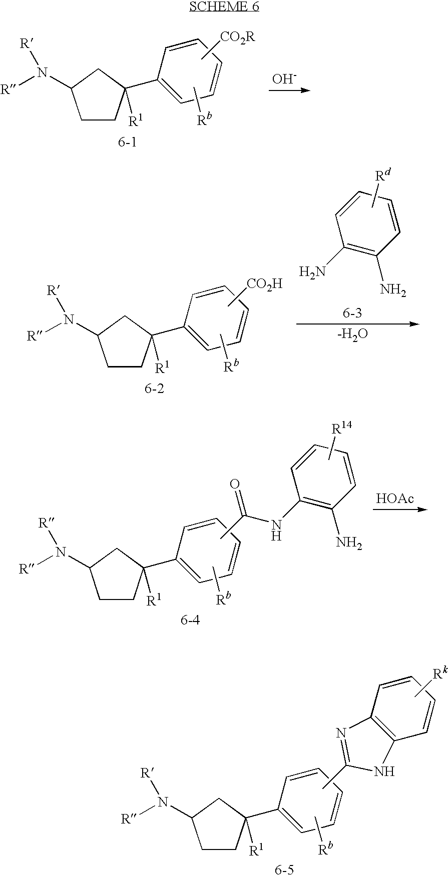

The chemokine modulators (6-5) is also prepared starting from the previously prepared amino ester (6-1). The amino ester (6-2) is hydrolyzed into the amino acid (6-2) which undergoes the coupling and cyclization to give the chemokine modulators (6-4) and (6-5).

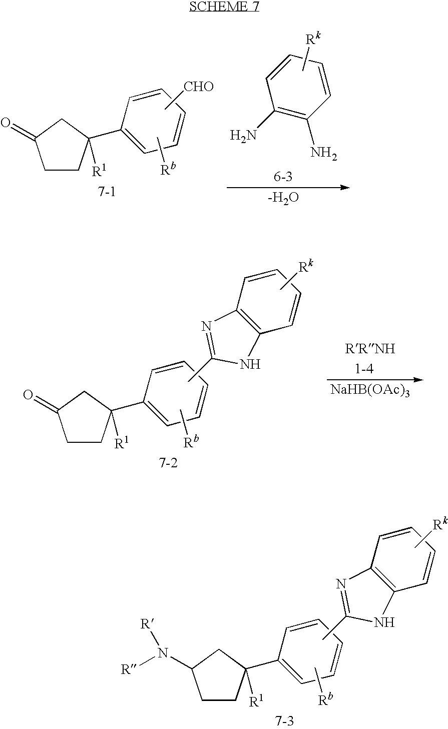

An alternative route is also developed starting from the ketoaldehyde (7-1), which is prepared according to the procedure in the Scheme 1. Direct oxidative condensation of the aldehyde with diamino benzene gives the keto imidazole (7-2). Various chemokine modulators (7-3) can be readily prepared by reductive amination.

Scheme 8 shows a route to non cylopentyl chemokine modulators. According to this route, amine 8-1 is first reductively alkylated with a tetrahydropyranone in the presence of a suitable base such as triethylamine and a reducing agent such as sodium triacetoxyborohydride or sodium cyanoborohydride in a suitable solvent such as DCM or methanol respectively. The resulting amine can then be further reductively alkylated with formaldehyde to give the amino-ester 8-2. Saponification of the ester functionality can be achieved with a base such as sodium hydroxide in a suitable aqueous solvent mixture. Condensation of the resulting acid (8-3) with a diamine of the form 8-4, in the presence of EDC, DMAP, and a abse such as triethylamine in DCM, followed by prolonged heating with acetic acid give chemokine modulators of the formula 8-5.

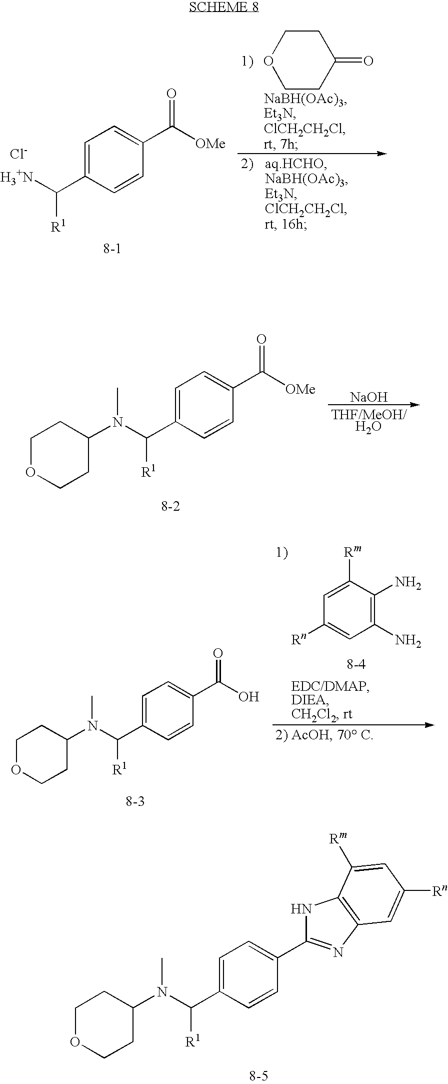

In cases where R^{m} or Rⁿ is a suitable electrophile, such as an aryl halide or triflate (X′), the chemokine modulator can be further modified to yield a new chemokine modulator such as 8-6, via a transition metal catalyzed cross coupling reaction. This route is shown in Scheme 9.

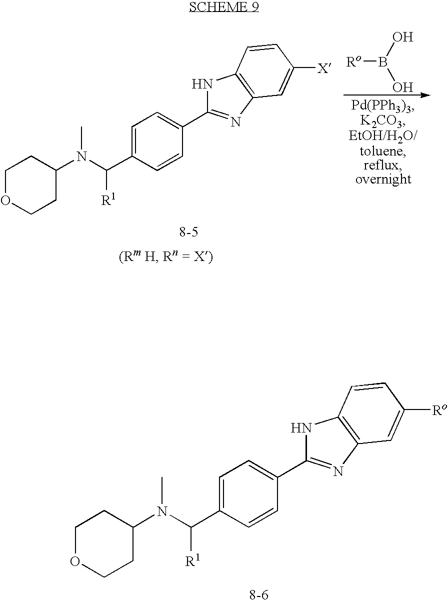

An alternate method for the preparation of non-cyclopentyl chemokine modulators is shown in Scheme 10. According to this route, the condensation reaction between the diamine (8-4) and the boc protected amino acid (8-7) gives the imidazole 8-8. Removal of the Boc protecting group can be accomplished using HCl to give the amine 8-9. Successive reductive alkylations of the amine with the pyranone and formalydehyde then gives chemokine modulator 8-10.

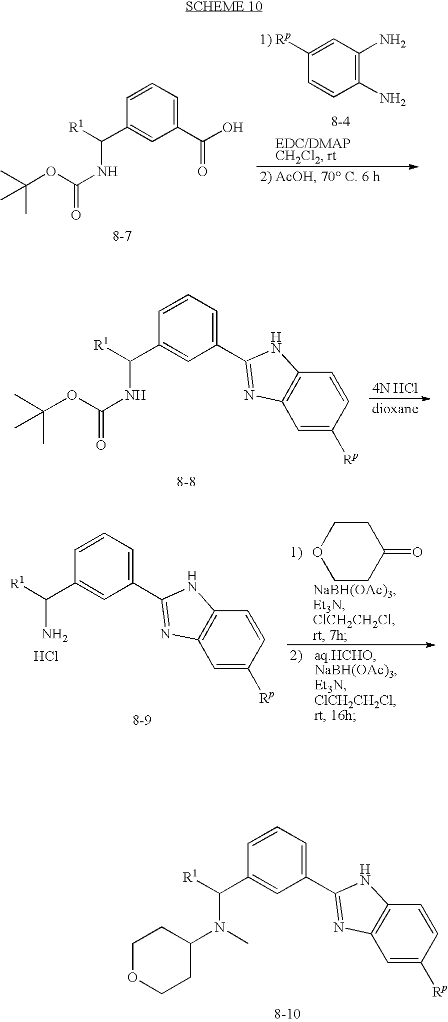

In some cases the order of carrying out the foregoing reaction schemes may be varied to facilitate the reaction or to avoid unwanted reaction products. The following examples are provided for the purpose of further illustration only and are not intended to be limitations on the disclosed invention.

Concentration of solutions was generally carried out on a rotary evaporator under reduced pressure. Flash chromatography was carried out on silica gel (230-400 mesh). NMR spectra were obtained in CDCl₃ solution unless otherwise noted. Coupling constants (J) are in hertz (Hz). Abbreviations: diethyl ether (ether), triethylamine (TEA), N,N-diisopropylethylamine (DMA) saturated aqueous (sat'd), room temperature (rt), hour(s) (h), minute(s) (min).

The following are representative Procedures for the preparation of the compounds used in the following Examples or which can be substituted for the compounds used in the following Examples which may not be commercially available.

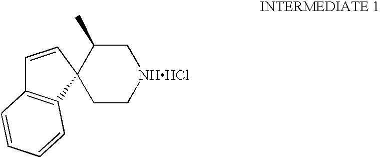

### Step A:

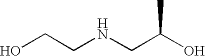

To a cooled (0° C.) solution of ethanolamine (41.8 g, 0.685 mol) in water (90 mL) was added neat (R)-propylene oxide (4.97 g, 85.6 mmol), dropwise. After 1 h at 0° C. the reaction was allowed to rise to rt and stirred overnight. The reaction mixture was concentrated at ˜80° C. in vacuo to remove the water and most of the ethanolamine, to give 11.79 g of crude product, containing some residual ethanolamine. This material was used without further purification in Step B.

### Step B:

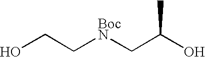

The diol prepared in Step A (11.8 g crude [˜86% pure], ca. 83 mmol) was dissolved in DCM (150 mL) and treated with Boc₂O (23.4 g, 107 mmol) in DCM (75 mL) over 15 min. The reaction mixture was stirred over the weekend, concentrated, and purified by MPLC, eluting with 5% MeOH/EtOAc to provide 14.8 g (81%) of product.

### Step C:

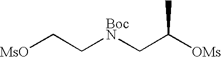

To a solution of the Boc-protected diol prepared in Step B (13.2 g, 60.3 mmol) and triethylamine (21.0 mL, 15.3 g, 151 mmol) in DCM (150 mL) at 0° C. was added dropwise methanesulfonyl chloride (9.56 mL, 14.1 g, 125 mmol). The reaction mixture was then stirred for 1.5 h, diluted with more DCM (100 mL) and washed with 3N HCl (250 mL). The aqueous layer was extracted again with DCM (200 mL), and the organic layers were combined and washed with 1N HCl (250 mL), saturated NaHCO₃ solution (250 mL), and brine (250 mL). The organic layer was dried over MgSO₄, filtered, and concentrated to give 22.8 g of crude bis-mesylate, which was used immediately. If not used immediately the bis-mesylate underwent decomposition.

### Step D:

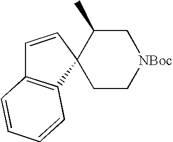

Indene (7.03 mL, 7.00 g, 60.3 mmol) was added dropwise over 4 min to a 1.0 M THF solution of LHMDS (127 mL, 127 mmol) at 0° C. After stirring for an additional 30 min, this solution was transferred via cannula to a solution of bis-mesylate (22.6 g, 60.3 mmol), prepared as described in Step C above, in THF (75 mL) at 0° C. The mixture was stirred for 2 h, warmed to rt and stirred overnight. The reaction mixture was partially concentrated and then partitioned between ethyl acetate and water. The organic layer was extracted again with ethyl acetate and the organic layers were combined. The organic phase was then washed with brine, dried over MgSO₄, filtered and concentrated to give 17.3 g of crude product. Purification by MPLC, eluting with 15% ethyl acetate/hexane, afforded 9.51 g (53%) of piperidine as a ˜3:1 mixture of trans to cis (determined by H NMR). The mixture was crystallized from hot hexane to give 6 g (33%) of pure trans isomer (>20:1 by H NMR).

H NMR (CDCl₃, 400 MHz): δ 7.29 (dt, J=6.4, 1.6 Hz, 1H), 7.20 (m, 3H), 6.83 (d, J=6.0 Hz, 1H), 6.67 (d, J=5.6 Hz, 1H), 4.20 (br s, 2H), 2.97 (br t, J=3.2 Hz, 1H), 2.69 (br t, J=2.4 Hz, 1H), 2.16 (m, 1H), 2.07 (dt, J=4.4, 13.2 Hz, 1H), 1.49 (s, 9H), 1.25 (m, 1H), 0.31 (d, J=6.8 Hz, 3H).

### Step E:

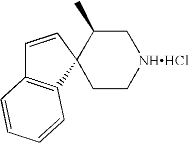

The Boc-piperidine prepared in Step D (4.35 g, 14.5 mmol) was dissolved in an anhydrous 4 N HCl solution in dioxane and stirred at rt for 1 h. The reaction mixture was then concentrated to afford 3.81 g of product.

EI-MS calc. for C14H17N: 199; Found: 200 (M)⁺.

### Example 1

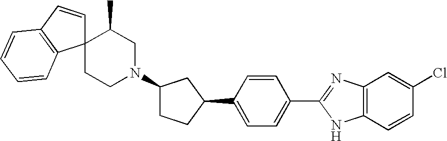

### Step A:

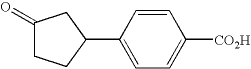

A mixture of cyclopentenone (6.5 g, 80 mmol), 4-carboxybenzene boric acid (15.0 g, 90 mmol), sodium acetate (16.4 g, 200 mmol), palladium acetate (2.30 g, 10 mmol), antimony trichloride (2.30 g, 10 mmol) in acetic acid (250 ml) was stirred over two days. The dark solid was removed by filtration and the filtrate was evaporated to remove acetic acid under reduced pressure. To the residue was added water (200 mL) and ethyl acetate (400 ml), stirred for 30 min. The organic phase was separated and washed with brine (200 ml), dried over anhydrous sodium sulfate, filtered and evaporated. The residue was chromatographed on silica gel (eluted with 25% ethyl acetate in hexane) to afford the title compound as a yellow solid (1.2 g). ¹H-NMR (CDCl₃, 300 MHz): δ 7.99 (d, J=8.3 Hz, 2H), 7.41 (d, J=8.2, 2H), 3.30 (m, 1H), 2.60 (m, 1H), 2.40 (m, 4H), 2.00 (m, 1H).

### Step B:

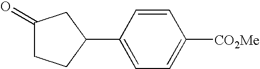

The acid (1.02 g, 5 mmol) from Step A immediately above, iodomethane (0.62 ml, 10 mmol) and potassium carbonate (1.38 g, 10 mmol) in DMF (20 ml) was stirred at RT overnight. The reaction mixture was diluted with 50 ml of water, extracted with 20% ethyl acetate/hexane (2×50 ml). The combined organic layers were washed with water (50 ml) and brine (50 ml), dried over anhydrous sodium sulfate, filtered and evaporated to afford a yellow solid (1.0 g). ¹H-NMR (CDCl₃, 300 MHz): δ 7.98 (d, J=8.4 Hz, 2H), 7.31 (d, J=8.4, 2H), 3.89 (s, 3H), 3.43 (m, 1H), 2.62 (dd, 1H), 2.40 (m, 4H), 2.00 (m, 1H).

### Step C:

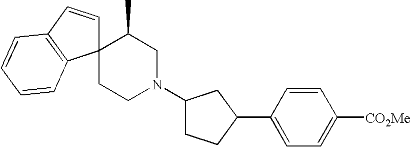

The cyclopentanone from Step B immediately above (220 mg, 1 mmol) was combined in DCM (20 mL) with 3-methylspiroindenepiperidine Intermediate 1 (236 mg, 1 mmol), triethylamine (195 mg, 1.5 mmol), sodium tiacetoxyborohydride (633 mg, 3 mmol), and molecular sieves (4A, 2.0 g). The resulting mixture was stirred at room temperature for 24 h. The reaction mixture was then filtered through a celite plug, washing with ethyl acetate. The filtrate was washed with saturated NaHCO₃ solution, then with brine, dried over anhydrous MgSO₄, filtered, and concentrated. Purification by preparative TLC (silica, 0.1/0.9/99 of NH₄OH/methanol/DCM) gave 280 mg of the title product.

ESI-MS calc. for C27H31NO2: 401; Found: 402 (M+H).

### Step D:

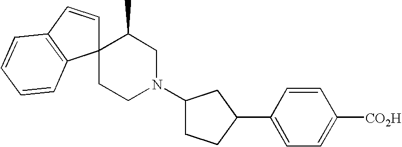

The ester from Step C immediately above (280 mg, 0.7 mmol) was combined in a mixture of THF and water (20 ml, 2:1 v/v) with lithium hydroxide monohydrate (82 mg, 2 mmol). The resulting mixture was stirred at room temperature for 16 h. The reaction mixture was condensed and purified by preparative TLC (silica, 1:9 of methanol/DCM) to give 200 mg of the title product amino acid.

ESI-MS calc. for C26H29N2O: 387; Found: 388 (M+H).

### Step E:

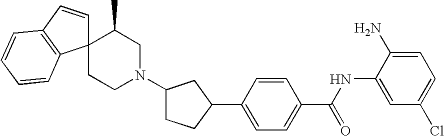

The amino acid from Step D immediately above (38.7 mg, 0.1 mmol) was combined in DCM (2 ml) with 4-chloro-1,2-phenylenediamine (28 mg, 0.2 mmol), EDAC (38 mg, 0.2 mmol) and DMAP (5 mg). The resulting mixture was stirred at room temperature for 16 h, loaded on preparative TLC (silica), developed with 0.1/0.9/99 of NH₄OH/methanol/DCM to give 25 mg of the title product aminoamide.

ESI-MS calc. for C32H34ClN3O: 511; Found: 512 (M+H).

### Step F:

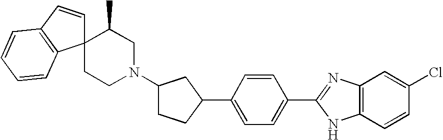

The aminoamide from Step E immediately above (20 mg) in 0.5 ml of acetic acid was heated at 60° C. overnight. The acetic acid was removed under reduced pressure and the residue was purified on preparative TLC (silica, 1/9/90 of NH₄OH/methanol/DCM) to give 12 mg of the title product aminoimidazole as a mixture of 4 diastereomers. Four respective single enantiomers were obtained by chiral HPLC (OD column, 10% ethanol/hexane).

ESI-MS calc. for C32H32ClN3: 493; Found: 494 (M+H).

### Example 2

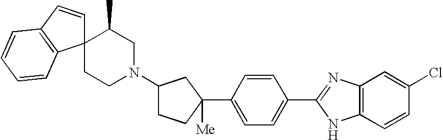

### Step A:

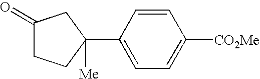

A mixture of 3-methyl-cyclopentenone (7.7 g, 80 mmol), 4-carboxybenzene boric acid (15.0 g, 90 mmol), sodium acetate (16.4 g, 200 mmol), palladium acetate (2.30 g, 10 mmol), antimony trichloride (2.30 g, 10 mmol) in acetic acid (250 ml) was stirred over two days. The dark solid was removed by filtration and the filtrate was evaporated to remove acetic acid under reduced pressure. To the residue was added water (200 mL) and ethyl acetate (400 ml), stirred for 30 min. The organic phase was separated and washed with brine (200 ml), dried over anhydrous sodium sulfate, filtered and evaporated. The residue was mixed with potassium carbonate (40 g) and iodomethane (10 ml) in DMF (100 ml), stirred overnight, diluted with water, extracted with 20% ethyl acetate/hexane, dried over sodium sulfate, evaporated. Purification on FC (10% ethyl acetate/hexane) gave the title compound (0.42 g). ¹H-NMR (CDCl₃, 300 MHz): δ 7.92 (d, J=8.5 Hz, 2H), 7.32 (d, J=8.5, 2H), 3.88 (s, 2H), 3.83 (s, 3H), 3.42 (m, 4H), 1.35 (s, 2H).

### Step B:

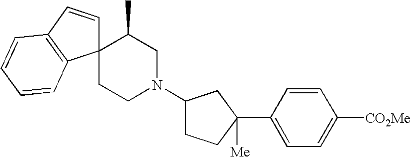

The cyclopentanone from Step A immediately above (233 mg, 1 mmol) was combined in DCM (20 mL) with 3-methylspiroindenepiperidine Intermediate 1 (236 mg, 1 mmol), triethylamine (195 mg, 1.5 mmol), sodium tiacetoxyborohydride (633 mg, 3 mmol), and molecular sieves (4A, 2.0 g). The resulting mixture was stirred at room temperature for 24 h. The reaction mixture was then filtered through a celite plug, washing with ethyl acetate. The filtrate was washed with saturated NaHCO₃ solution, then with brine, dried over anhydrous MgSO₄, filtered, and concentrated. Purification by preparative TLC (silica, 0.1/0.9/99 of NH₄OH/methanol/DCM) gave 120 mg of the title product. ESI-MS calc. for C28H33NO2: 415; Found: 416 (M+H).

### Step C:

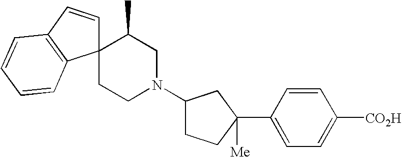

The ester from Step B immediately above (120 mg) was combined in a mixture of THF and water (20 ml, 2:1 v/v) with lithium hydroxide monohydrate (41 mg, 1 mmol). The resulting mixture was stirred at room temperature for 16 h. The reaction mixture was condensed and purified by preparative TLC (silica, 1:9 of methanol/DCM) to give 120 mg of the title product amino acid as a white solid.

ESI-MS calc. for C27H31N2O: 401; Found: 402 (M+H).

### Step D:

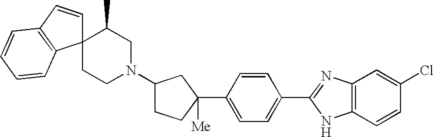

The amino acid from Step C immediately above (120 mg, 0.3 mmol) was combined in DCM (2 ml) with 4-chloro-1,2-phenylenediamine (142 mg, 1.0 mmol), EDAC (191 mg, 1.0 mmol) and DMAP (5 mg). The resulting mixture was stirred at room temperature for 16 h, loaded on preparative TLC (silica), developed with 0.1/0.9/99 of NH₄OH/methanol/DCM to give 110 mg of the corresponding amide. This material was heated with acetic acid (0.5 ml) at 60° C. overnight, evaporated to remove acetic acid. The residue was purified on preparative TLC (silica, 1/9/90 of NH₄OH/methanol/DCM) to give 80 mg of the title compound as a brown solid.

ESI-MS calc. for C33H34ClN3: 508; Found: 509 (M+H).

### Example 3

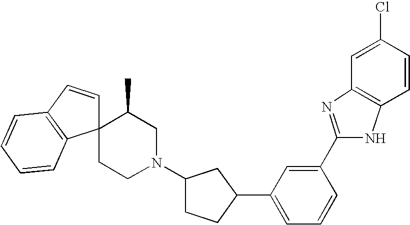

### Step A:

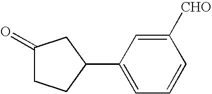

A mixture of cyclopentenone (10.0 g, 120 mmol), 3-formylbenzene boric acid (15.0 g, 100 mmol), sodium acetate (16.4 g, 200 mmol), palladium acetate (2.30 g, 10 mmol), antimony trichloride (2.30 g, 10 mmol) in acetic acid (500 ml) was stirred over two days. The dark solid was removed by filtration and the filtrate was evaporated to remove acetic acid under reduced pressure. To the residue was added water (200 mL) and ethyl acetate (400 ml), stirred for 30 min. The organic phase was separated and washed with brine (200 ml), dried over anhydrous sodium sulfate, filtered and evaporated. The residue was chromatographed on silica gel (eluted with 25% ethyl acetate in hexane) to afford the title compound as a yellow oil (5.2 g). ¹H-NMR (CDCl₃, 300 MHz): δ 9.96 (s, 1H), 7.75 (m, 3H), 7.49 (m, 3H), 3.44 (m, 1H), 2.68 (m, 1H), 2.42 (m, 2H), 2.30 (m, 2H), 2.00 (m, 1H).

### Step B:

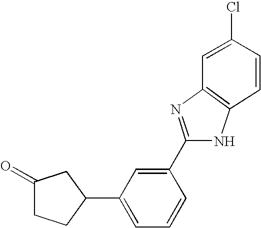

The aldehyde (150 mg, 0.8 mmol) from Step A immediately above was heated at 65° C. for 20 min with sodium bisulfite (200 mg) in methanol (10 ml), then 3-chloro-1,2-phenylenediamine (120 mg, 0.8 mmol) was added. The mixture was stirred at 65° C. for one hour, diluted with ethyl acetate (50 ml), washed with sat. aq. sodium bicarbonate, water and then brine, dried over anhydrous sodium sulfate, evaporated, purified on preparative TLC (50% ethyl acetate/hexane) to give 156 mg of the title compound as a yellow gummy solid.

ESI-MS calc. for C18H15ClN2O: 310; Found: 311 (M+H).

### Step C:

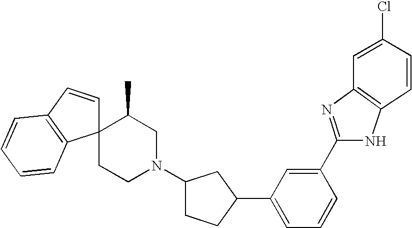

The ketone from Step B immediately above (155 mg, 0.5 mmol) was combined in DCM (20 mL) with 3-methylspiroindenepiperidine Intermediate 1 (140 mg, 0.5 mmol), triethylamine (129 mg, 1.0 mmol), sodium tiacetoxyborohydride (411 mg, 2.0 mmol), and molecular sieves (4A, 2.0 g). The resulting mixture was stirred at room temperature for 24 h. The reaction mixture was then filtered through a celite plug, washing with ethyl acetate. The filtrate was washed with saturated NaHCO₃ solution, then with brine, dried over anhydrous MgSO₄, filtered, and concentrated. Purification by preparative TLC (silica, 0.1/0.9/99 of NH₄OH/methanol/DCM) gave 90 mg of the title product.

ESI-MS calc. for C32H32ClN3: 494; Found: 495 (M+H).

### Example 4

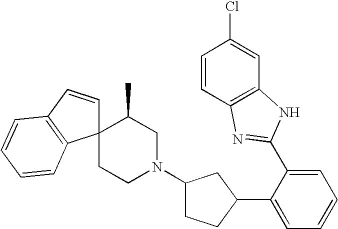

### Step A:

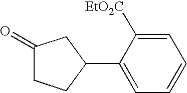

To a thick wall pressure tube was added ethyl 2-bromobenzoate (5.0 g, 21.8 mmol), cyclopentenone (5.5 ml, 61.5 mmol), triethyl amine (4.56 ml, 32.7 mmol), palladium acetate (48.9 mg, 0.218 mmol) and triphenyl phosphine (114.4 mg, 0.436). The tube was capped and stirred in 100° C. oil bath for 30 h. TLC showed the reaction was almost complete. The entire mixture was loaded on silica gel column without any workup, eluted with 30% ethyl acetate in hexane to afford 1.101 g of the title compound (second major spot on TLC). ¹H-NMR (CDCl₃, 300 MHz): δ 7.85 (m, 1H), 7.49 (m, 1H), 7.38 (m, 1H), 7.28 (m, 1H), 4.35 (q, J=7.14, 2H), 4.25 (m, 1H), 2.70 (m, 1H), 2.25-2.50 (m, 4H), 2.00 (m, 1H), 1.40 (t, J=7.14, 3H).

### Step B:

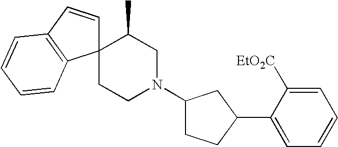

The cyclopentanone from Step A immediately above (900 mg, 3.873 mmol) was combined in DCM (50 mL) with 3-methylspiroindenepiperidine Intermediate 1 (1.096 g, 4.648 mmol), DIEA (0.816 ml, 4.684 mmol), sodium tiacetoxyborohydride (3.284 g, 15.49 mmol), and molecular sieves (4A, 5.0 g). The resulting mixture was stirred at room temperature for 24 h. The reaction mixture was then filtered through a celite plug, washing with methanol. The filtrates were concentrated and purified on FC (silica, 10% in DCM) to give 1.517 g (66%) of the title product as an oil.

ESI-MS calc. for C28H33NO2: 415; Found: 416 (M+H).

### Step C:

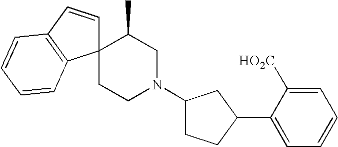

The ester from Step C immediately above (1.51 g, 3.64 mmol) was combined in a mixture of dioxane (10 ml), ethanol (5 ml) and water (5 ml) with lithium hydroxide monohydrate (0.917 g, 21.83 mmol). The resulting mixture was stirred at 70° C. for 15 h. The reaction mixture was condensed to dryness and purified on FC (silica, 20% methanol/DCM) to give two fractions (cis: faster isomer: 412.7 mg+trans: slower isomer: 391.1 mg) of the title product amino acid. Both fractions showed the same LC-MS data.

ESI-MS calc. for C26H29NO2: 387; Found: 388 (M+H).

### Step D:

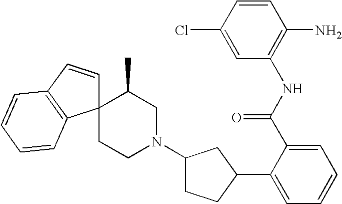

The cis amino acid from Step C immediately above (faster isomer, 50 mg, 0.129 mmol) was combined in DCM (2 ml) with 4-chloro-1,2-phenylenediamine (55.2 mg, 0.387 mmol), EDAC (123.6 mg, 0.647 mmol) and DMAP (3.1 mg). The resulting mixture was stirred at room temperature for 16 h, condensed and loaded on preparative TLC (silica), developed with 0.1/0.9/99 of NH₄OH/methanol/DCM to give the title product aminoamide.

ESI-MS calc. for C32H34ClN3O: 511; Found: 512 (M+H).

### Step F:

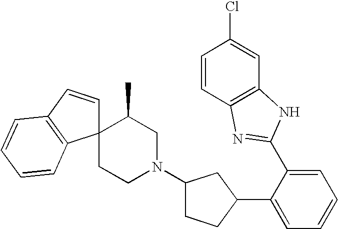

The entire aminoamide from Step E immediately above in 3 ml of acetic acid was heated at 100° C. for two days. The acetic acid was removed under reduced pressure and the residue was purified on preparative TLC (silica, 100% ethyl acetate) to give 16.6 mg of the title product aminoimidazole as a mixture of 2 cis diastereomers.

ESI-MS calc. for C32H32ClN3: 493; Found: 494 (M+H).

The similar procedure starting from trans amino acid from Step C immediately above (slower isomer, 50 mg, 0.129 mmol) gave the title aminoimidazole as a mixture of 2 trans diastereomers.

ESI-MS calc. for C32H32ClN3: 493; Found: 494 (M+H).

### Example 5

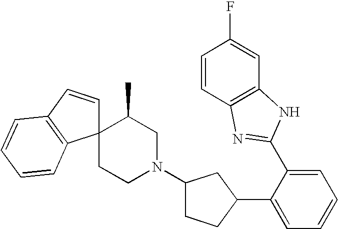

### Step A:

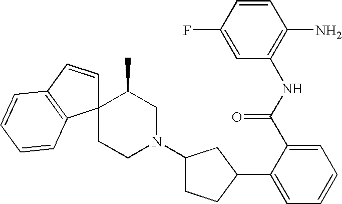

The cis amino acid from Step C of Example 4 (faster isomer, 48.8 mg, 0.129 mmol) was combined in DCM (2 ml) with 4-fluoro-1,2-phenylenediamine (55.2 mg, 0.387 mmol), EDAC (123.6 mg, 0.647 mmol) and DMAP (3.1 mg). The resulting mixture was stirred at room temperature for 16 h, condensed and loaded on preparative TLC (silica), developed with 0.1/0.9/99 of NH₄OH/methanol/DCM to give the title product aminoamide.

ESI-MS calc. for C32H34FN3O: 495; Found: 496 (M+H).

### Step B

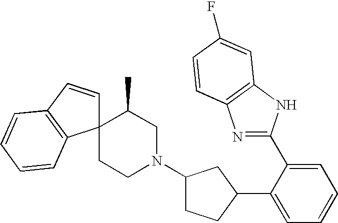

The entire aminoamide from Step A immediately above in 3 ml of acetic acid was heated at 100° C. for two days. The acetic acid was removed under reduced pressure and the residue was purified on preparative TLC (silica, 100% ethyl acetate) to give 16.6 mg of the title product aminoimidazole as a mixture of 2 cis diastereomers.

ESI-MS calc. for C32H32FN3: 477; Found: 478 (M+H).

The similar procedure starting from trans amino acid from Step C of Example 4 (slower isomer, 50 mg, 0.129 mmol) gave the title aminoimidazole as a mixture of 2 trans diastereomers. ESI-MS calc. for C32H32FN3: 477; Found: 478 (M+H).

### Example 6

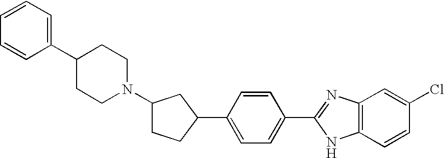

### Step A:

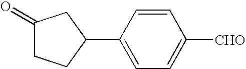

A mixture of cyclopentenone (10.0 g, 120 mmol), 4-formylbenzene boric acid (15.0 g, 100 mmol), sodium acetate (16.4 g, 200 mmol), palladium acetate (4.60 g, 20 mmol), antimony trichloride (4.60 g, 20 mmol) in acetic acid (500 ml) was stirred over two days. The dark solid was removed by filtration and the filtrate was evaporated to remove acetic acid under reduced pressure. To the residue was added water (200 mL) and ethyl acetate (400 ml), stirred for 30 min. The organic phase was separated and washed with brine (200 ml), dried over anhydrous sodium sulfate, filtered and evaporated. The residue was chromatographed on silica gel (eluted with 30% ethyl acetate in hexane) to afford the title compound as a yellow oil (8.7 g).

### Step B:

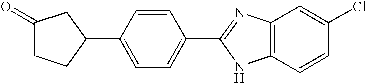

The aldehyde (3.0 g, 16 mmol) from Step A immediately above was heated at 65° C. for 60 min with sodium bisulfite (4.0 g) in methanol (100 ml), then 3-chloro-1,2-phenylenediamine (2.4 mg, 16 mmol) was added. The mixture was stirred at 65° C. for one hour, diluted with ethyl acetate (50 ml), washed with sat. aq. sodium bicarbonate, water and then brine, dried over anhydrous sodium sulfate, evaporated give 5.3 g of the title compound as a yellow solid.

ESI-MS calc. for C18H15ClN2O: 310; Found: 311 (M+H).

### Step C:

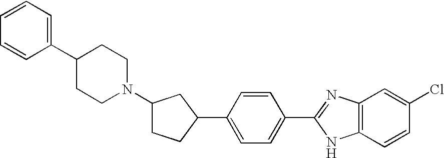

The ketone from Step B immediately above (155 mg, 0.5 mmol) was combined in DCM (20 mL) with 3-methylspiroindenepiperidine Intermediate 1 (140 mg, 0.5 mmol), triethylamine (129 mg, 1.0 mmol), sodium tiacetoxyborohydride (411 mg, 2.0 mmol), and molecular sieves (4A, 2.0 g). The resulting mixture was stirred at room temperature for 24 h. The reaction mixture was then filtered through a celite plug, washing with ethyl acetate. The filtrate was washed with saturated NaHCO₃ solution, then with brine, dried over anhydrous MgSO₄, filtered, and concentrated. Purification by preparative TLC (silica, 0.1/0.9/99 of NH₄OH/methanol/DCM) gave 74 mg of the title product.

ESI-MS calc. for C29H30ClN3: 457; Found: 458 (M+H).

### Example 7

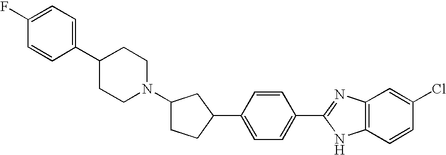

The ketone from Step B of Example 6 (155 mg, 0.5 mmol) was combined in DCM (20 mL) with 4-fluorophenylpiperidine hydrochloride (220 mg, 1.0 mmol), triethylamine (260 mg, 2.0 mmol), sodium tiacetoxyborohydride (411 mg, 2.0 mmol), and molecular sieves (4A, 1.0 g). The resulting mixture was stirred at room temperature for 24 h. The reaction mixture was then filtered through a celite plug, washing with ethyl acetate. The filtrate was washed with saturated NaHCO₃ solution, then with brine, dried over anhydrous MgSO₄, filtered, and concentrated. Purification by preparative TLC (silica, 0.1/0.9/99 of NH₄OH/methanol/DCM) gave 54 mg of the title product.

ESI-MS calc. for C29H29ClFN3: 474; Found: 475 (M+H).

### Example 8

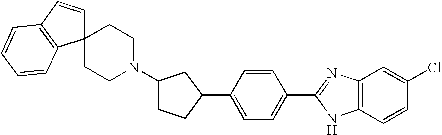

The ketone from Step B of Example 6 (155 mg, 0.5 mmol) was combined in DCM (20 mL) with spiroindene hydrochloride (120 mg, 0.5 mmol), triethylamine (129 mg, 1.0 mmol), sodium tiacetoxyborohydride (411 mg, 2.0 mmol), and molecular sieves (4A, 1.0 g). The resulting mixture was stirred at room temperature for 24 h. The reaction mixture was then filtered through a celite plug, washing with ethyl acetate. The filtrate was washed with saturated NaHCO₃ solution, then with brine, dried over anhydrous MgSO₄, filtered, and concentrated. Purification by preparative TLC (silica, 0.1/0.9/99 of NH₄OH/methanol/DCM) gave 77 mg of the title product.

ESI-MS calc. for C31H30ClN3: 479; Found: 480 (M+H).

### Example 9

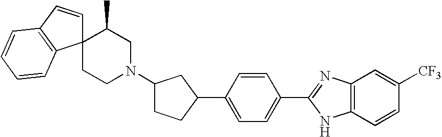

The amino acid from Step C of Example 1 (100 mg, 0.25 mmol) was combined in DCM (2 ml) with 4-trifluoromethyl-1,2-phenylenediamine (88 mg, 0.5 mmol), EDAC (191 mg, 1.0 mmol) and DMAP (5 mg). The resulting mixture was stirred at room temperature for 16 h, loaded on preparative TLC (silica), developed with 0.1/0.9/99 of NH₄OH/methanol/DCM to give the corresponding amide. This material was heated with acetic acid (0.5 ml) at 60° C. overnight, evaporated to remove acetic acid. The residue was purified on preparative TLC (silica, 1/9/90 of NH₄OH/methanol/DCM) to give 59 mg of the title compound aminoimidazole as a mixture of 4 diastereomers. Four respective single enantiomers were obtained by chiral HPLC (OD column, 10% ethanol/hexane).

ESI-MS calc. for C33H32F3N3: 527; Found: 528 (M+H).

### Example 10

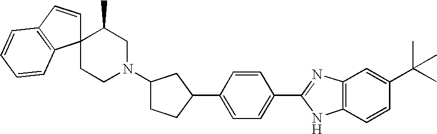

The amino acid from Step C of Example 1 (38.7 mg, 0.1 mmol) was combined in DCM (1 ml) with 4-tert-butyl-1,2-phenylenediamine (164 mg, 0.5 mmol), EDAC (95 mg, 0.5 mmol) and DMAP (5 mg). The resulting mixture was stirred at room temperature for 16 h, loaded on preparative TLC (silica), developed with 0.1/0.9/99 of NH₄OH/methanol/DCM to give the corresponding amide. This material was heated with acetic acid (0.5 ml) at 60° C. overnight, evaporated to remove acetic acid. The residue was purified on preparative TLC (silica, 1/9/90 of NH₄OH/methanol/DCM) to give 23 mg of the title compound aminoimidazole as a mixture of 4 diastereomers.

ESI-MS calc. for C36H41N3: 515; Found: 516 (M+H).

### Example 11

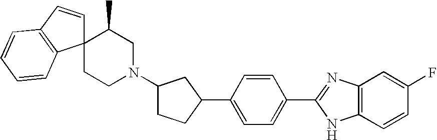

The amino acid from Step C of Example 1 (100 mg, 0.25 mmol) was combined in DCM (2 ml) with 4-fluoro-1,2-phenylenediamine (80 mg, 0.5 mmol), EDAC (191 mg, 1.0 mmol) and DMAP (5 mg). The resulting mixture was stirred at room temperature for 16 h, loaded on preparative TLC (silica), developed with 0.1/0.9/99 of NH₄OH/methanol/DCM to give the corresponding amide. This material was heated with acetic acid (0.5 ml) at 60° C. overnight, evaporated to remove acetic acid. The residue was purified on preparative TLC (silica, 1/9/90 of NH₄OH/methanol/DCM) to give 50 mg of the title compound aminoimidazole as a mixture of 4 diastereomers. Four respective single enantiomers were obtained by chiral HPLC (OD column, 10% ethanol/hexane).

ESI-MS calc. for C32H32FN3: 477; Found: 478 (M+H).

### Example 12

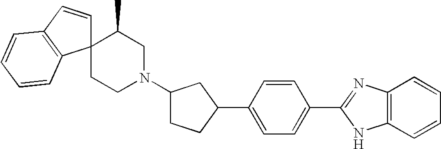

The amino acid from Step C of Example 1 (100 mg, 0.25 mmol) was combined in DCM (2 ml) with 1,2-phenylenediamine (108 mg, 1.0 mmol), EDAC (191 mg, 1.0 mmol) and DMAP (5 mg). The resulting mixture was stirred at room temperature for 16 h, loaded on preparative TLC (silica), developed with 0.1/0.9/99 of NH₄OH/methanol/DCM to give the corresponding amide. This material was heated with acetic acid (0.5 ml) at 60° C. overnight, evaporated to remove acetic acid. The residue was purified on preparative TLC (silica, 1/9/90 of NH₄OH/methanol/DCM) to give 52 mg of the title compound aminoimidazole as a mixture of 4 diastereomers. Four respective single enantiomers were obtained by chiral HPLC (OD column, 10% ethanol/hexane).

ESI-MS calc. for C32H33N3: 459; Found: 460 (M+H).

### Example 13

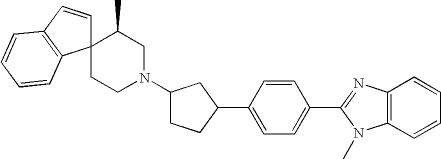

The amino acid from Step C of Example 1 (38.7 mg, 0.1 mmol) was combined in DCM (1 ml) with 1-amino-2-methylaminobenzene dihydrochloride (195 mg, 1.0 mmol), triethylamine (260 mg, 2 mmol), EDAC (191 mg, mmol) and DMAP (5 mg). The resulting mixture was stirred at room temperature for 16 h, loaded on preparative TLC (silica), developed with 0.1/0.9/99 of NH₄OH/methanol/DCM to give the corresponding amide. This material was heated with acetic acid (0.5 ml) at 60° C. overnight, evaporated to remove acetic acid. The residue was purified on preparative TLC (silica, 1/9/90 of NH₄OH/methanol/DCM) to give 32 mg of the title compound aminoimidazole as a mixture of 4 diastereomers.

ESI-MS calc. for C33H35N3: 473; Found: 474 (M+H).

### Example 14

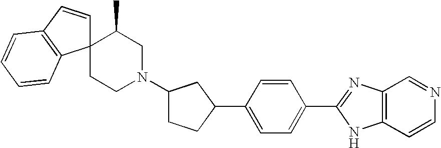

The amino acid from Step C of Example 1 (100 mg, 0.258 mmol) was combined in DCM (3 ml) with 3,4-diaminopyridine (84.5 mg, 0.76 mmol), triethylamine (260 mg, 2 mmol), EDAC (99 mg, mmol) and DMAP (3 mg). The resulting mixture was stirred at room temperature for 16 h, loaded on preparative TLC (silica), developed with 0.1/0.9/99 of NH₄OH/methanol/DCM to give 5 mg of the corresponding amide. This material was heated with acetic acid (0.5 ml) at 60° C. overnight, evaporated to remove acetic acid. The residue was purified on preparative TLC (silica, 1/9/90 of NH₄OH/methanol/DCM) to give 2.5 mg of the title compound aminoimidazole as a mixture of 4 diastereomers.

ESI-MS calc. for C31H32N4: 460; Found: 461 (M+H).

### Example 15

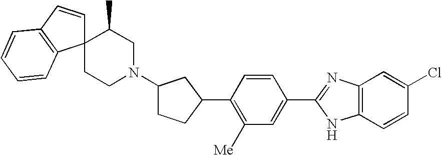

### Step A:

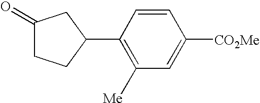

To a thick wall pressure tube was added methyl 4-bromo-3-methyl benzoate (5.0 g, 21.8 mmol), cyclopentenone (5.479 ml, 61.5 mmol), triethyl amine (4.65 ml, 32.7 mmol), palladium acetate (48.9 mg, 0.218 mmol) and triphenyl phosphine (114.4 mg, 0.436). The tube was capped and stirred in 100° C. oil bath for 30 h. TLC showed the reaction was almost complete. The entire mixture was loaded on silica gel column without any workup, eluted with 30% ethyl acetate in hexane to afford 2.16 g of the title compound (second major spot on TLC). ¹H-NMR (CDCl₃, 300 MHz): δ 7.85 (m, 2H), 7.26 (m, 1H), 3.89 (s, 3H), 3.62 (m, 1H), 2.65 (m, 1H), 2.40 (s, 3H), 2.25-2.50 (m, 4H), 2.00 (m, 1H).

### Step B:

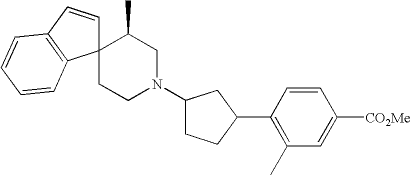

The cyclopentanone from Step A immediately above (1.0 g, 4.3 mmol) was combined in DCM (50 mL) with 3-methylspiroindenepiperidine Intermediate 1 (1.217 g, 5.16 mmol), DIEA (0.899 ml, 5.16 mmol), sodium tiacetoxyborohydride (2.735 g, 12.9 mmol), and molecular sieves (4A, 5.0 g). The resulting mixture was stirred at room temperature for 24 h. The reaction mixture was then filtered through a celite plug, washing with methanol. The filtrates were concentrated and purified on FC (silica, 100%

ethyl acetate) to give 1.1846 g (66%) of the title product.

ESI-MS calc. for C28H33NO2: 415; Found: 416 (M+H).

### Step C:

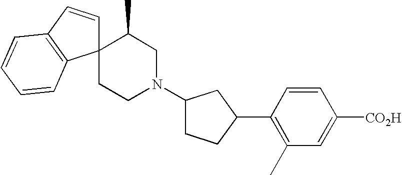

The ester from Step C immediately above (1.10 g, 2.647 mmol) was combined in a mixture of dioxane (20 ml), ethanol (5 ml) and water (10 ml) with lithium hydroxide monohydrate (0.667 g, 2 mmol). The resulting mixture was stirred at 60° C. for 4 h. The reaction mixture was condensed to dryness and purified on FC (silica, 50% methanol/DCM) to give 1.07 g (100%) of the title product amino acid.

ESI-MS calc. for C27H31NO2: 401; Found: 402 (M+H).

### Step D:

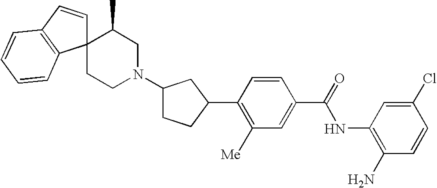

The amino acid from Step C immediately above (100 mg, 0.249 mmol) was combined in DCM (2 ml) with 4-chloro-1,2-phenylenediamine (106.5 mg, 0.747 mmol), EDAC (238.7 mg, 1.245 mmol) and DMAP (7 mg). The resulting mixture was stirred at room temperature for 16 h, condensed and loaded on preparative TLC (silica), developed with 0.1/0.9/99 of NH₄OH/methanol/DCM to give 153.2 mg of the title product aminoamide

ESI-MS calc. for C33H36ClN3O: 526; Found: 527 (M+H).

### Step F:

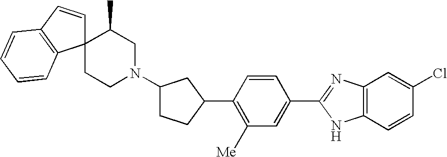

The aminoamide from Step E immediately above (130 mg, 0.249 mmol) in 3 ml of acetic acid was heated at 60° C. overnight. The acetic acid was removed under reduced pressure and the residue was purified on preparative TLC (silica, 1/9/90 of NH₄OH/methanol/DCM) to give 64.2 mg of the title product aminoimidazole as a mixture of 4 diastereomers.

ESI-MS calc. for C33H34ClN3: 507; Found: 508 (M+H).

### Example 16

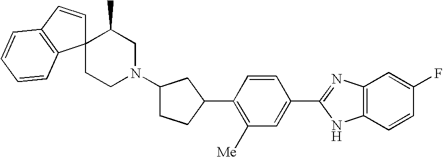

### Step A:

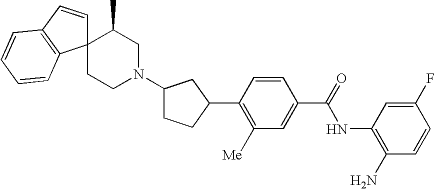

The amino acid from Step C of Example 15 (100 mg, 0.249 mmol) was combined in DCM (2 ml) with 4-fluoro-1,2-phenylenediamine (94.2 mg, 0.747 mmol), EDAC (238.7 mg, 1.245 mmol) and DMAP (7 mg). The resulting mixture was stirred at room temperature for 16 h, condensed and loaded on preparative TLC (silica), developed with 0.1/0.9/99 of NH₄OH/methanol/DCM to give 147.5 mg of the title product aminoamide.

ESI-MS calc. for C33H36FN3O: 509; Found: 510 (M+H).

### Step B:

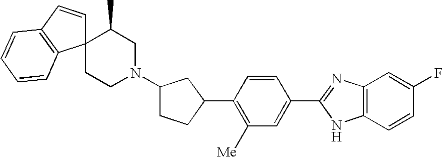

The aminoamide from Step A immediately above (127 mg, 0.249 mmol) in 3 ml of acetic acid was heated at 60° C. overnight. The acetic acid was removed under reduced pressure and the residue was purified on preparative TLC (silica, 1/9/90 of NH₄OH/methanol/DCM) to give 55.9 mg of the title product aminoimidazole as a mixture of 4 diastereomers.

ESI-MS calc. for C33H34FN3: 491; Found: 492 (M+H).

### Example 17

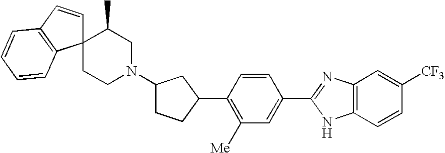

### Step A:

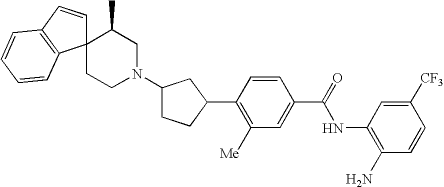

The amino acid from Step C of Example 15 (100 mg, 0.249 mmol) was combined in DCM (2 ml) with 4-trifluoromethyl-1,2-phenylenediamine (131.6 mg, 0.747 mmol), EDAC (238.7 mg, 1.245 mmol) and DMAP (7 mg). The resulting mixture was stirred at room temperature for 16 h, condensed and loaded on preparative TLC (silica), developed with 0.1/0.9/99 of NH₄OH/methanol/DCM to give 147.5 mg of the title product aminoamide.

ESI-MS calc. for C34H36F3N3O: 559; Found: 560 (M+H).

### Step B:

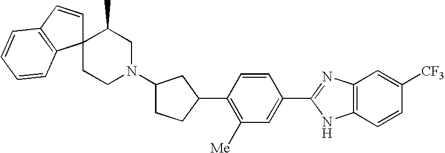

The aminoamide from Step A immediately above (139 mg, 0.249 mmol) in 3 ml of acetic acid was heated at 60° C. overnight. The acetic acid was removed under reduced pressure and the residue was purified on preparative TLC (silica, 1/9/90 of NH₄OH/methanol/DCM) to give 63.9 mg of the title product aminoimidazole as a mixture of 4 diastereomers.

ESI-MS calc. for C34H34F3N3: 541; Found: 542 (M+H).

### Example 18

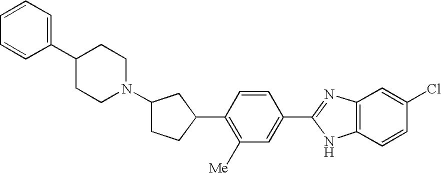

### Step A:

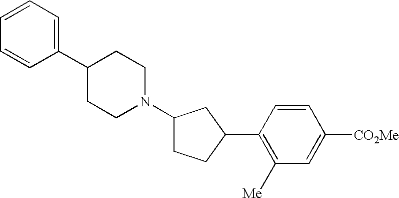

The cyclopentanone from Step A of Example 15 (1.0 g, 4.3 mmol) was combined in DCM (50 mL) with 4-phenylpiperidine hydrochloride (1.02 g, 5.16 mmol), DIEA (0.899 ml, 5.16 mmol), sodium triacetoxyborohydride (2.735 g, 12.9 mmol), and molecular sieves (4A, 5.0 g). The resulting mixture was stirred at room temperature for 24 h. The reaction mixture was then filtered through a celite plug, washing with methanol. The filtrates were concentrated and purified on FC (silica, 100% ethyl acetate) to give 1.323 g (81%) of the title product.

ESI-MS calc. for C25H31NO2: 377; Found: 378 (M+H).

### Step B:

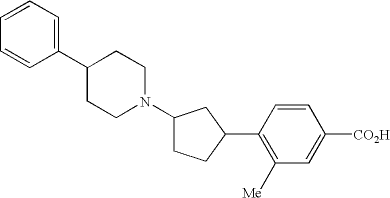

The ester from Step A immediately above (1.30 g, 3.444 mmol) was combined in a mixture of dioxane (15 ml), ethanol (5 ml) and water (5 ml) with lithium hydroxide monohydrate (0.579 g, 2 mmol). The resulting mixture was stirred at 60° C. for 4 h. The reaction mixture was condensed to dryness and purified on FC (silica, 50% methanol/DCM) to give 1.347 g (100%) of the title product amino acid.

ESI-MS calc. for C24H29NO2: 363; Found: 364 (M+H).

### Step C:

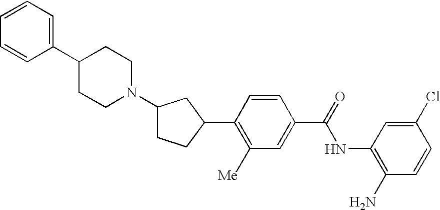

The amino acid from Step B immediately above (100 mg, 0.275 mmol) was combined in DCM (2 ml) with 4-chloro-1,2-phenylenediamine (117.6 mg, 0.825 mmol), EDAC (263.6 mg, 1.375 mmol) and DMAP (7 mg). The resulting mixture was stirred at room temperature for 16 h, condensed and loaded on preparative TLC (silica), developed with 0.1/0.9/99 of NH₄OH/methanol/DCM to give 81.5 mg of the title product aminoamide.

ESI-MS calc. for C30H34ClN3O: 487; Found: 488 (M+H).

### Step D:

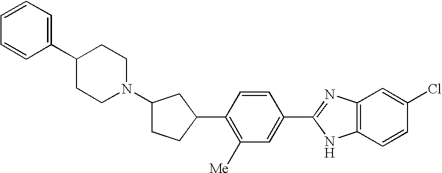

The aminoamide from Step C immediately above (81 mg) in 3 ml of acetic acid was heated at 60° C. overnight. The acetic acid was removed under reduced pressure and the residue was purified on preparative TLC (silica, 1/9/90 of NH₄OH/methanol/DCM) to give 28.8 mg of the title product aminoimidazole as a mixture of 4 diastereomers.

ESI-MS calc. for C30H32ClN3: 469; Found: 470 (M+H).

### Example 19

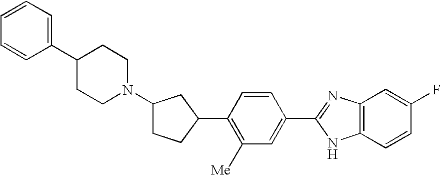

### Step A:

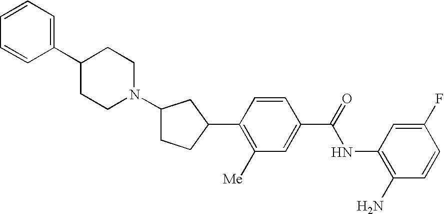

The amino acid from Step B of Example 18 (100 mg, 0.275 mmol) was combined in DCM (2 ml) with 4-fluoro-1,2-phenylenediamine (104.1 mg, 0.825 mmol), EDAC (263.6 mg, 1.375 mmol) and DMAP (7 mg). The resulting mixture was stirred at room temperature for 16 h, condensed and loaded on preparative TLC (silica), developed with 0.1/0.9/99 of NH₄OH/methanol/DCM to give 77.3 mg of the title product aminoamide.

ESI-MS calc. for C30H34FN3O: 471; Found: 472 (M+H).

### Step B:

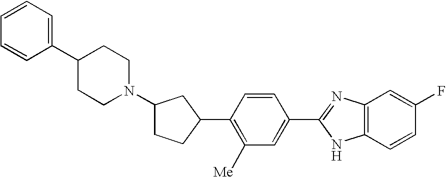

The aminoamide from Step A immediately above (77 mg, 0.164) in 3 ml of acetic acid was heated at 60° C. overnight. The acetic acid was removed under reduced pressure and the residue was purified on preparative TLC (silica, 1/9/90 of NH₄OH/methanol/DCM) to give 31.6 mg of the title product aminoimidazole as a mixture of 4 diastereomers.

ESI-MS calc. for C30H32FN3: 453; Found: 454 (M+H).

### Example 20

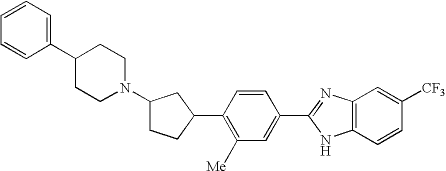

### Step A:

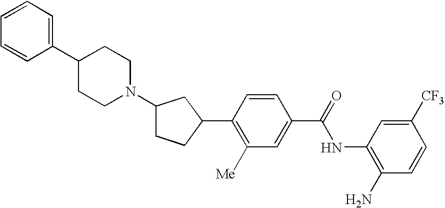

The amino acid from Step B of Example 18 (100 mg, 0.275 mmol) was combined in DCM (2 ml) with 4-fluoro-1,2-phenylenediamine (145.3 mg, 0.825 mmol), EDAC (263.6 mg, 1.375 mmol) and DMAP (7 mg). The resulting mixture was stirred at room temperature for 16 h, condensed and loaded on preparative TLC (silica), developed with 0.1/0.9/99 of NH₄OH/methanol/DCM to give 79.5 mg of the title product aminoamide.

ESI-MS calc. for C31H34F3N3O: 521; Found: 522 (M+H).

### Step B:

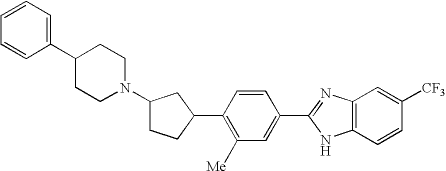

The aminoamide from Step A immediately above (77 mg, 0.164) in 3 ml of acetic acid was heated at 60° C. overnight. The acetic acid was removed under reduced pressure and the residue was purified on preparative TLC (silica, 1/9/90 of NH₄OH/methanol/DCM) to give 31.6 mg of the title product aminoimidazole as a mixture of 4 diastereomers.

ESI-MS calc. for C31H32F3N3: 503; Found: 504 (M+H).

### Example 21

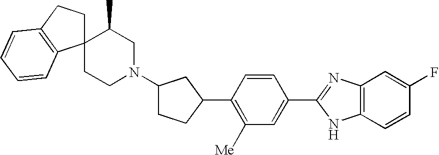

The example 16 (25 mg, 0.0443 mmol) was dissolved in EtOH (5 ml) and 10% Pd/C (5 mg) was added. Hydrogenation was carried out with a hydrogen balloon for 2 h, the catalyst was removed by filtration and the filtrate was concentrated in vacuum to give 21.5 mg of the title product.

ESI-MS calc. for C33H36FN3: 493; Found: 494 (M+H).

### Example 22

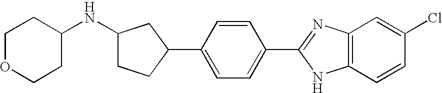

The ketone from Step B of Example 6 (155 mg, 0.5 mmol) was combined in DCM (20 mL) with 4-tetrahydropyranyl amine hydrochloride (136 mg, 1.0 mmol), triethylamine (129 mg, 1.0 mmol), sodium tiacetoxyborohydride (411 mg, 2.0 mmol), and molecular sieves (4A, 2.0 g). The resulting mixture was stirred at room temperature for 24 h. The reaction mixture was then filtered through a celite plug, washing with ethyl acetate. The filtrate was washed with saturated NaHCO₃ solution, then with brine, dried over anhydrous MgSO₄, filtered, and concentrated. Purification by preparative TLC (silica, 0.1/0.9/99 of NH₄OH/methanol/DCM) gave 74 mg of the title product.

ESI-MS calc. for C23H6ClN3O: 395; Found: 396 (M+H).

### Example 23

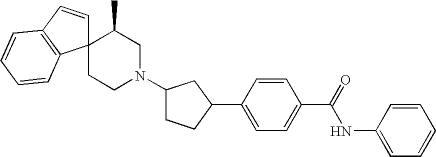

The amino acid from Step D of Example 1 (20 mg, 0.0514 mmol) was combined in DCM (0.5 ml) with aniline (70 mg, 0.753 mmol), EDAC (70 mg, 0.366 mmol) and DMAP (5 mg). The resulting mixture was stirred at room temperature for 16 h, loaded on preparative TLC (silica) and developed with 100% ethyl acetate to give 24 mg of the title product aminoamide.

ESI-MS calc. for C32H34N2O: 462; Found: 463 (M+H).

### Example 24

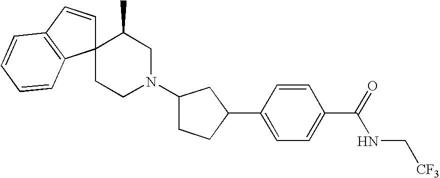

The amino acid from Step D of Example 1 (50 mg, 0.13 mmol) was combined in DCM (0.5 ml) with 2,2,2-trifluoroethylamine hydrochloride (35.2 mg, 0.39 mmol), EDAC (74.2 mg, 0.366 mmol). The resulting mixture was stirred at room temperature for 16 h, loaded on preparative TLC (silica) and developed with 10% MeOH in DCM to give 28.7 mg of the title product.

ESI-MS calc. for C28H31F3N2O: 468; Found: 469 (M+H).

### Example 25

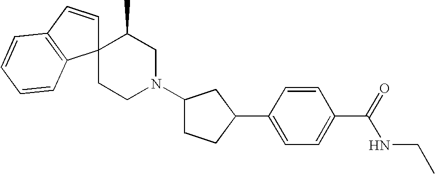

The amino acid from Step D of Example 1 (50 mg, 0.13 mmol) was combined in DCM (0.5 ml) with ethylamine hydrochloride (21 mg, 0.39 mmol), EDAC (74.2 mg, 0.366 mmol). The resulting mixture was stirred at room temperature for 16 h, loaded on preparative TLC (silica) and developed with 10% MeOH in DCM to give 28.7 mg of the title product.

ESI-MS calc. for C28H34N2O: 414; Found: 415 (M+H).

### Example 26

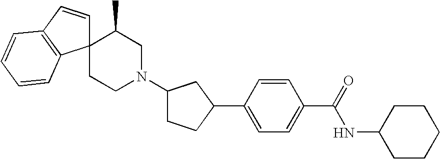

The amino acid from Step D of Example 1 (38.7 mg, 0.1 mmol) was combined in DCM (1.0 ml) with cyclohexylamine (20 mg, 0.2 mmol), EDAC (100 mg, 0.52 mmol) and DMAP (5 mg). The resulting mixture was stirred at room temperature for 16 h, loaded on preparative TLC (silica) and developed with 100% ethyl acetate to give 37 mg of the title product aminoamide.

ESI-MS calc. for C32H40N2O: 468; Found: 469 (M+H).

### Example 27

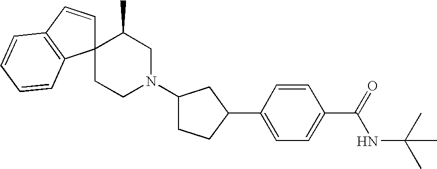

The amino acid from Step D of Example 1 (38.7 mg, 0.1 mmol) was combined in DCM (1.0 ml) with tert-butylamine (36.5 mg, 0.5 mmol), EDAC (191 mg, 1.0 mmol) and 4N HCl in dioxane (0.1 ml). The resulting mixture was stirred at room temperature for 16 h, loaded on preparative TLC (silica) and developed with 10% MeOH in DCM to give 15 mg of the title product aminoamide.

ESI-MS calc. for C30H38N2O: 442; Found: 443 (M+H).

### Example 28

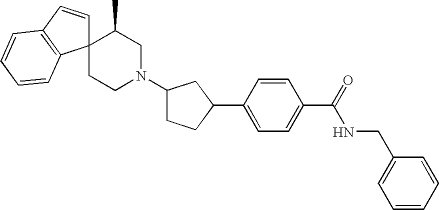

The amino acid from Step D of Example 1 (50 mg, 0.13 mmol) was combined in DCM (1.0 ml) with benzylamine (28.4 mg, 0.26 mmol), EDAC (74.2 mg, 0.26 mmol) and 4N HCl in dioxane (0.065 ml, 0.26 mmol). The resulting mixture was stirred at room temperature for 16 h, loaded on preparative TLC (silica) and developed with 10% MeOH in DCM to give 32 mg of the title product.

ESI-MS calc. for C33H36N2O: 476; Found: 477 (M+H).

### Example 29

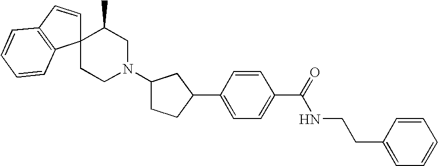

The amino acid from Step D of Example 1 (50 mg, 0.13 mmol) was combined in DCM (1.0 ml) with phenylethylamine (32.0 mg, 0.26 mmol), EDAC (74.2 mg, 0.26 mmol) and 4N HCl in dioxane (0.065 ml, 0.26 mmol). The resulting mixture was stirred at room temperature for 16 h, loaded on preparative TLC (silica) and developed with 10% MeOH in DCM to give 29.8 mg of the title product.

ESI-MS calc. for C34H38N2O: 490; Found: 491 (M+H).

### Example 30

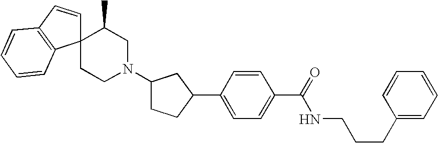

The amino acid from Step D of Example 1 (50 mg, 0.13 mmol) was combined in DCM (1.0 ml) with phenylpropylamine (35.2 mg, 0.26 mmol), EDAC (74.2 mg, 0.26 mmol) and 4N HCl in dioxane (0.065 ml, 0.26 mmol). The resulting mixture was stirred at room temperature for 16 h, loaded on preparative TLC (silica) and developed with 10% MeOH in DCM to give 36.0 mg of the title product.

ESI-MS calc. for C35H40N2O: 504; Found: 505 (M+H).

### Example 31

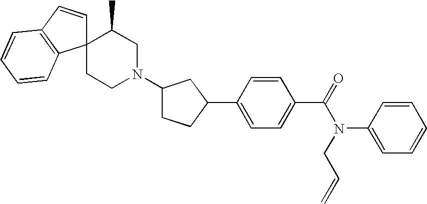

The amino acid from Step D of Example 1 (50 mg, 0.13 mmol) was combined in DCM (1.0 ml) with N-allylaniline (34.6 mg, 0.26 mmol), EDAC (74.2 mg, 0.26 mmol) and 4N HCl in dioxane (0.065 ml, 0.26 mmol). The resulting mixture was stirred at room temperature for 16 h, loaded on preparative TLC (silica) and developed with 10% MeOH in DCM to give 54.0 mg of the title product.

ESI-MS calc. for C35H38N2O: 502; Found: 503 (M+H).

### Example 32

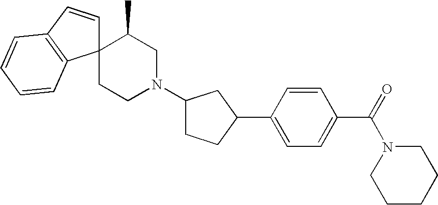

The amino acid from Step D of Example 1 (50 mg, 0.13 mmol) was combined in DCM (1.0 ml) with piperidine (0.026 ml, 0.26 mmol), EDAC (74.2 mg, 0.26 mmol) and 4N HCl in dioxane (0.065 ml, 0.26 mmol). The resulting mixture was stirred at room temperature for 16 h, loaded on preparative TLC (silica) and developed with 10% MeOH in DCM to give 54.0 mg of the title product.

ESI-MS calc. for C31H38N₂O: 454; Found: 455 (M+H).

### Example 33

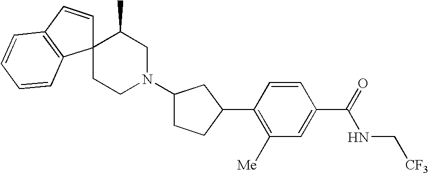

The amino acid from Step C of Example 15 (50 mg, 0.1245 mmol) was combined in DCM (1.0 ml) with 2,2,2-trifluoroethylamine hydrochloride (33.7 mg, 0.249 mmol), EDAC (119.3 mg, 0.623 mmol). The resulting mixture was stirred at room temperature for 16 h, loaded on preparative TLC (silica) and developed with 7% MeOH in DCM to give 49.7 mg of the title product.

ESI-MS calc. for C29H33F3N2O: 482; Found: 483 (M+H).

### Example 34

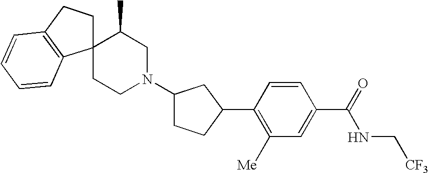

The example 33 (25 mg, 0.048 mmol) was dissolved in EtOH (5 ml) and 10% Pd/C (5 mg) was added. Hydrogenation was carried out with a hydrogen balloon for 2 h, the catalyst was removed by filtration and the filtrate was concentrated in vacuum to give 26.9 mg of the title product.

ESI-MS calc. for C29H35F3N2O: 484; Found: 485 (M+H).

### Example 35

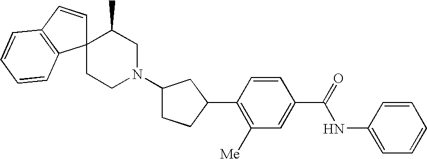

The amino acid from Step C of Example 15 (50 mg, 0.1245 mmol) was combined in DCM (1.0 ml) with aniline (0.024 ml, 0.249 mmol), EDAC (119.3 mg, 0.623 mmol). The resulting mixture was stirred at room temperature for 16 h, loaded on preparative TLC (silica) and developed with 7% MeOH in DCM to give 57.5 mg of the title product.

ESI-MS calc. for C33H36N2O: 476; Found: 477 (M+H).

### Example 36

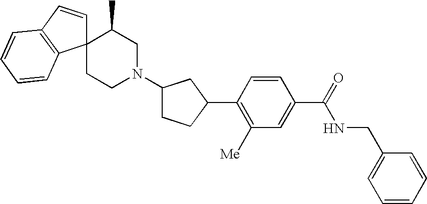

The amino acid from Step C of Example 15 (50 mg, 0.1245 mmol) was combined in DCM (1.0 ml) with benzyl amine (0.027 ml, 0.249 mmol), EDAC (119.3 mg, 0.623 mmol). The resulting mixture was stirred at room temperature for 16 h, loaded on preparative TLC (silica) and developed with 10% MeOH in DCM to give 52 mg of the title product.

ESI-MS calc. for C34H38N2O: 490; Found: 491 (M+H).

### Example 37

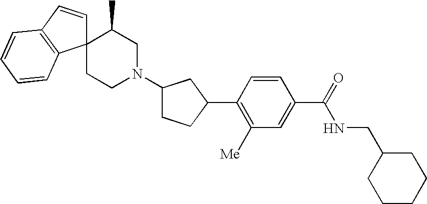

The amino acid from Step C of Example 15 (50 mg, 0.1245 mmol) was combined in DCM (1.0 ml) with c-hexyl methylamine (0.032 ml, 0.249 mmol), EDAC (119.3 mg, 0.623 mmol). The resulting mixture was stirred at room temperature for 16 h, loaded on preparative TLC (silica) and developed with 10% MeOH in DCM to give 57 mg of the title product.

ESI-MS calc. for C34H44N2O: 496; Found: 497 (M+H).

### Example 38

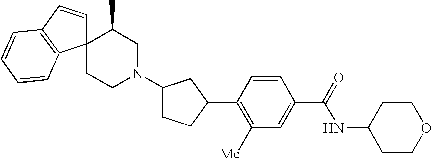

The amino acid from Step C of Example 15 (30 mg, 0.0747 mmol) was combined in DCM (1.0 ml) with tetrahydropyranyl amine hydrochloride (20.5 mg, 0.149 mmol), EDAC (57 mg, 0.299 mmol) and DMAP (2 mg). The resulting mixture was stirred at room temperature for 16 h, loaded on preparative TLC (silica) and developed with 10% MeOH in DCM to give 27 mg of the title product.

ESI-MS calc. for C32H40N2O2: 484; Found: 485 (M+H).

### Example 39

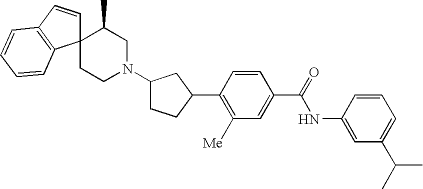

The amino acid from Step C of Example 15 (30 mg, 0.0747 mmol) was combined in DCM (1.0 ml) with 3-isopropylaniline (20.4 mg, 0.149 mmol), EDAC (57 mg, 0.299 mmol) and DMAP (2 mg). The resulting mixture was stirred at room temperature for 16 h, loaded on preparative TLC (silica) and developed with 10% MeOH in DCM to give 34 mg of the title product.

ESI-MS calc. for C36H42N2O: 518; Found: 519 (M+H).

### Example 40

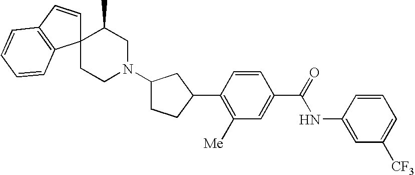

The amino acid from Step C of Example 15 (30 mg, 0.0747 mmol) was combined in DCM (1.0 ml) with 3-trifluoromethyl aniline (24 mg, 0.149 mmol), EDAC (57 mg, 0.299 mmol) and DMAP (2 mg). The resulting mixture was stirred at room temperature for 16 h, loaded on preparative TLC (silica) and developed with 10% MeOH in DCM to give 32 mg of the title product.

ESI-MS calc. for C34H35F3N2O: 544; Found: 545 (M+H).

### Example 41

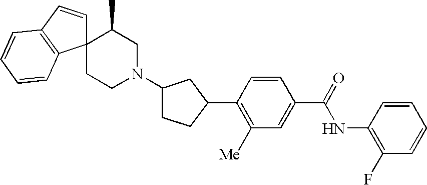

The amino acid from Step C of Example 15 (30 mg, 0.0747 mmol) was combined in DCM (1.0 ml) with 2-fluoroaniline (0.022 ml, 0.22 mmol), EDAC (57 mg, 0.299 mmol) and DMAP (2 mg). The resulting mixture was stirred at room temperature for 16 h, loaded on preparative TLC (silica) and developed with 10% MeOH in DCM to give 22.5 mg of the title product.

ESI-MS calc. for C33H35FN2O: 494; Found: 495 (M+H).

### Example 42

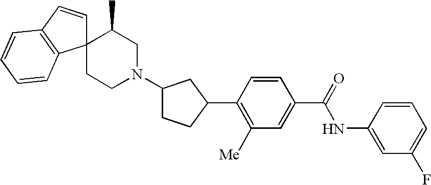

The amino acid from Step C of Example 15 (30 mg, 0.0747 mmol) was combined in DCM (1.0 ml) with 2-fluoroaniline (0.022 ml, 0.22 mmol), EDAC (57 mg, 0.299 mmol) and DMAP (2 mg). The resulting mixture was stirred at room temperature for 16 h, loaded on preparative TLC (silica) and developed with 10% MeOH in DCM to give 30.8 mg of the title product.

ESI-MS calc. for C33H35FN2O: 494; Found: 495 (M+H).

### Example 43

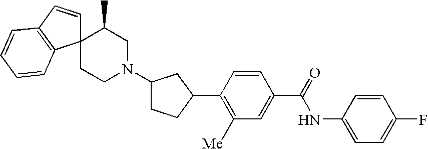

The amino acid from Step C of Example 15 (30 mg, 0.0747 mmol) was combined in DCM (1.0 ml) with 2-fluoroaniline (0.022 ml, 0.22 mmol), EDAC (57 mg, 0.299 mmol) and DMAP (2 mg). The resulting mixture was stirred at room temperature for 16 h, loaded on preparative TLC (silica) and developed with 10% MeOH in DCM to give 33.7 mg of the title product.

ESI-MS calc. for C33H35FN2O: 494; Found: 495 (M+H).

### Example 44

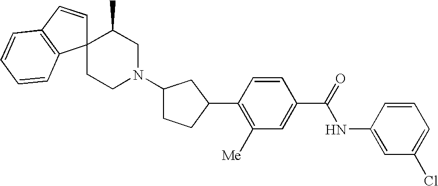

The amino acid from Step C of Example 15 (30 mg, 0.0747 mmol) was combined in DCM (1.0 ml) with 2-fluoroaniline (0.024 ml, 0.22 mmol), EDAC (57 mg, 0.299 mmol) and DMAP (2 mg). The resulting mixture was stirred at room temperature for 16 h, loaded on preparative TLC (silica) and developed with 10% MeOH in DCM to give 31 mg of the title product.

ESI-MS calc. for C33H35ClN2O: 510; Found: 511 (M+H).

### Example 45

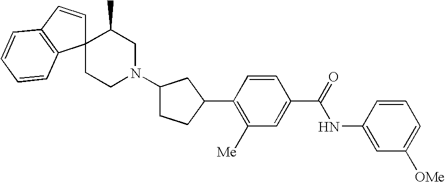

The amino acid from Step C of Example 15 (30 mg, 0.0747 mmol) was combined in DCM (1.0 ml) with 2-fluoroaniline (0.024 ml, 0.26 mmol), EDAC (57 mg, 0.299 mmol) and DMAP (2 mg). The resulting mixture was stirred at room temperature for 16 h, loaded on preparative TLC (silica) and developed with 10% MeOH in DCM to give 51 mg of the title product.

ESI-MS calc. for C34H38N2O2: 506; Found: 507 (M+H).

### Example 46

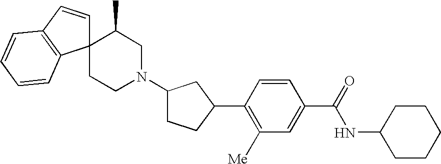

The amino acid from Step C of Example 15 (30 mg, 0.0747 mmol) was combined in DCM (1.0 ml) with 2-fluoroaniline (0.026 ml, 0.26 mmol), EDAC (57 mg, 0.299 mmol) and DMAP (2 mg). The resulting mixture was stirred at room temperature for 16 h, loaded on preparative TLC (silica) and developed with 10% MeOH in DCM to give 25.7 mg of the title product.

ESI-MS calc. for C33H42N2O: 482; Found: 483 (M+H).

### Example 47

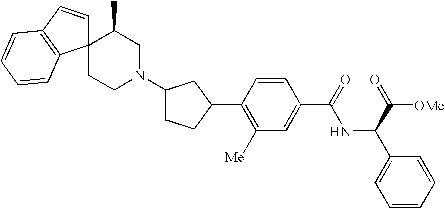

The amino acid from Step C of Example 15 (50 mg, 0.1245 mmol) was combined in DCM (1.0 ml) with (R)-(−)-2-phenylglycine methyl ester hydrochloride (37.7 mg, 0.1868 mmol), EDAC (119.7 mg, 0.299 mmol). The resulting mixture was stirred at room temperature for 16 h, loaded on preparative TLC (silica) and developed with 10% MeOH in DCM to give 58.5 mg of the title product.

ESI-MS calc. for C36H40N2O3: 548; Found: 549 (M+H).

### Example 48

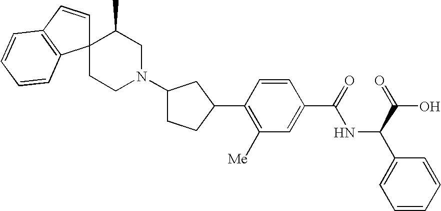

The amino ester (Example 47, 40 mg) was combined in a mixture of ethanol (1 ml) and water (0.5 ml) with lithium hydroxide monohydrate (20 mg). The resulting mixture was stirred at room temperature for 4 h, evaporated to dryness. The residue was dissolved in methanol, filtered through silica gel plug, washed with methanol, concentrated to dryness to give a white solid (41 mg).

ESI-MS calc. for C35H38N2O3: 534; Found: 535 (M+H).

### Example 49

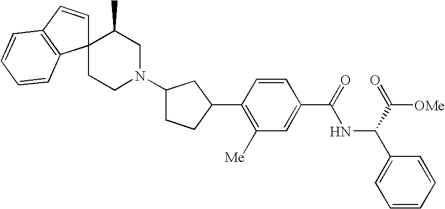

The amino acid from Step C of Example 15 (50 mg, 0.1245 mmol) was combined in DCM (1.0 ml) with (S)-(+)-2-phenylglycine methyl ester hydrochloride (37.7 mg, 0.1868 mmol), EDAC (119.7 mg, 0.299 mmol). The resulting mixture was stirred at room temperature for 16 h, loaded on preparative TLC (silica) and developed with 10% MeOH in DCM to give 43.2 mg of the title product.

ESI-MS calc. for C36H40N2O3: 548; Found: 549 (M+H).

### Example 50

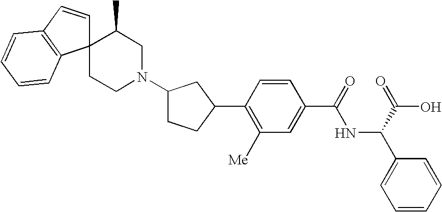

The amino ester (Example 47, 40 mg) was combined in a mixture of ethanol (1 ml) and water (0.5 ml) with lithium hydroxide monohydrate (20 mg). The resulting mixture was stirred at room temperature for 4 h, evaporated to dryness. The residue was dissolved in methanol, filtered through silica gel plug, washed with methanol, concentrated to dryness to give a white solid (42 mg).

ESI-MS calc. for C35H38N2O3: 534; Found: 535 (M+H).

### Example 51

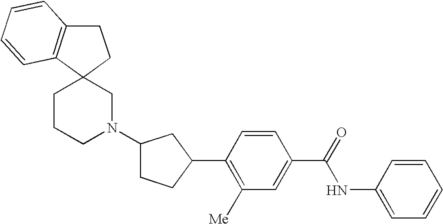

### Step A:

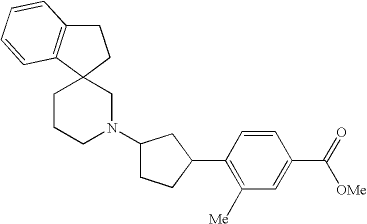

The cyclopentanone from Step A of Example 15 (100 mg, 0.43 mmol) was combined in DCM (5 mL) with 3-spiroindanepiperidine Intermediate hydrochloride (115.5 mg, 0.516 mmol), DMA (0.090 ml, 0.516 mmol), sodium triacetoxyborohydride (364.6 mg, 1.72 mmol), and molecular sieves (4A, 500 mg). The resulting mixture was stirred at room temperature for 24 h. The reaction mixture was then filtered through a celite plug, washing with methanol. The filtrates were concentrated and purified on FC (silica, 10% in DCM) to give 163.1 mg of the title product. ESI-MS calc. for C27H33NO2: 403; Found: 404 (M+H).

### Step B:

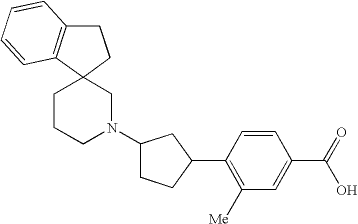

The amino ester from Step A immediately above (1.50 mg, 0.372 mmol) was combined in a mixture of ethanol (4 ml) and water (2 ml) with lithium hydroxide monohydrate (94 mg, 2.23 mmol). The resulting mixture was stirred at RT overnight. The reaction mixture was condensed to dryness and purified on FC (silica, 20% methanol/DCM) to give 68.5 mg of the title product.

ESI-MS calc. for C26H31NO2: 389; Found: 390 (M+H).

### Step C:

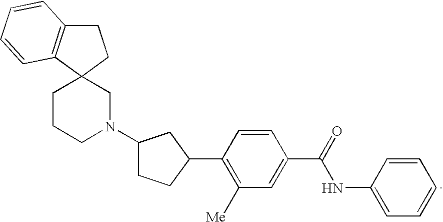

The amino acid from Step B immediately above (60 mg, 0.154 mmol) was combined in DCM (2 ml) with aniline (0.042 ml, 0.462 mmol), EDAC (148 mg, 0.77 mmol) and DMAP (4 mg). The resulting mixture was stirred at room temperature for 16 h, condensed and loaded on preparative TLC (silica), developed with 0.1/0.9/99 of NH₄OH/methanol/DCM to give 63.2 mg of the title product.

ESI-MS calc. for C32H36N2O: 464; Found: 465 (M+H).

### Example 52

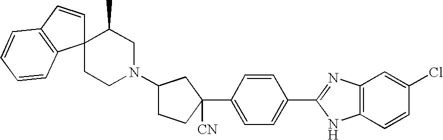

### Step A:

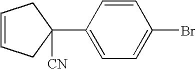

A mixture of 4-bromophenylacetonitrile (39.2 g, 0.2 mol), LiH (4.0 g, 0.5 mol), cis-1,4-dichloro-2-butene (23.6 g, 0.225 mol) in DME/DMU (9:1, 400 ml) was heated at 60° C. overnight, cooled at RT, poured into ice-water, extracted with 20% ethyl acetate/hexane. The organic phase was washed with water, dried over sodium sulfate, filtered and evaporated. The residue was purified by FC (silica gel, 20% ethyl acetate/hexane) to afford 42 g of the title product as light yellow oil. ¹H-NMR (CDCl₃, 300 MHz): δ 7.49 (d, J=1.93, 2H), 7.40 (d, J=1.93, 1H), 5.80 (s, 2H), 3.32 (d, J=14.12, 2H), 2.87 (d, J=14.12, 2H), 1H).

### Step B:

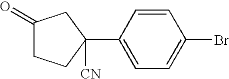

To a stirred solution of cyclopentane from Step A immediately above in ether (70 ml) at 0° C. was added borane-THF (1.0 M, 35 ml, 35 mmol) slowly. The mixture was stirred at room temperature for 3 h, diluted with methylene dichloride (600 ml), then added magnesium sulfate (24 g) and PCC (74 g, 342 mmol). The mixture was stirred overnight, dumped the solution on a silica gel column, eluted with 30% ethyl acetate in hexane to give 4.65 g of the title compound. ¹H-NMR (CDCl₃, 300 MHz): δ 7.60 (m, 2H), 7.32 (d, J=8.45 Hz, 1H), 7.12 (d, J=8.45 Hz, 1H), 3.72 (m, 1H), 3.10 (m, 1H), 1.80-2.90 (m, 5H).

### Step C:

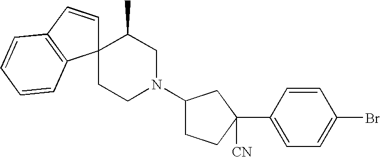

The cyclopentanone from Step B immediately above (2.83 g, 10.7 mmol) was combined in DCM (80 mL) with 3-methylspiroindenepiperidine Intermediate 1 (2.52 g, 10.7 mmol), DIEA (1.86 ml, 10.7 mmol), sodium tiacetoxyborohydride (4.537 g, 21.4 mmol), and molecular sieves (4A, 5.0 g). The resulting mixture was stirred at room temperature for 24 h. The reaction mixture was then filtered through a celite plug, washing with methanol. The filtrates were concentrated and purified on FC (silica, 40% ethyl acetate in hexane) to give 1.5446 g (32%) of the title product.

ESI-MS calc. for C26H27BrN2: 446; Found: 447 (M+H).

### Step D:

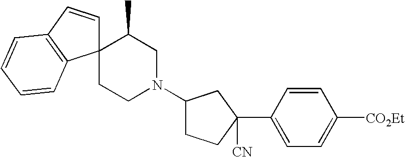

The phenylbromide from Step C immediately above (918 mg, 2.05 mmol) was combined in a mixture of triethylamine (249 mg, 2.46 mmol), palladium chloride (36.3 mg, 0.21 mmol), triphenylphosphine (107.5 mg, 0.41 mmol) in ethanol (20 ml). The mixture vacuum and then flushed with carbon monoxide. The procedure was repeated three times and the mixture was then stirred under atmosphere of carbon monoxide at 100° C. for 3 days. After filtered off the solid catalyst, the solution was evaporated to dryness. The residue was purified on preparative TLC (silica gel, 10% MeOH/DCM) to afford 389.7 mg (43%) of the title product.

ESI-MS calc. for C29H32N2O2: 440; Found: 441 (M+H).

### Step E:

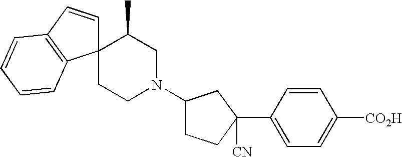

The ester from Step C immediately above (369 mg, 0.838 mmol) was combined in a mixture of dioxane (8 ml) and water (4 ml) with lithium hydroxide monohydrate (140.8 mg, 3.352 mmol). The resulting mixture was stirred at RT for 4 h. The reaction mixture was condensed to dryness and purified on preparative TLC (silica, 10% methanol/DCM) to give 278 mg (80%) of the title product amino acid.

ESI-MS calc. for C27H28N2O2: 412; Found: 413 (M+H).

### Step F:

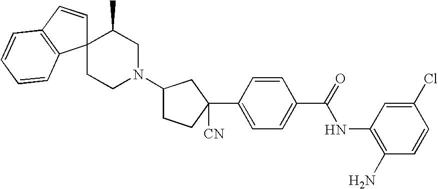

The amino acid from Step E immediately above (100 mg, 0.242 mmol) was combined in DCM (2 ml) with 4-chloro-1,2-phenylenediamine (103.5 mg, 0.726 mmol), EDAC (139.2 mg, 0.726 mmol) and DMAP (5 mg). The resulting mixture was stirred at room temperature for 16 h, condensed and loaded on preparative TLC (silica), developed with 0.1/0.9/99 of NH₄OH/methanol/DCM to give 105.0 mg (81%) of the title product.

ESI-MS calc. for C33H33ClN4O: 536; Found: 537 (M+H).

### Step G:

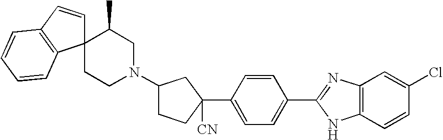

The aminoamide from Step F immediately above (100 mg, 0.249 mmol) in 2 ml of acetic acid was heated at 60° C. overnight. The acetic acid was removed under reduced pressure and the residue was purified on preparative TLC (silica, 1/9/90 of NH₄OH/methanol/DCM) to give 56.5 mg (51%) of the title product aminoimidazole as a mixture of 4 diastereomers.

ESI-MS calc. for C33H31ClN4: 518; Found: 519 (M+H).

### Example 53

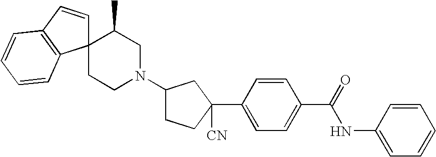

The amino acid from Step E of Example 52 (50 mg, 0.121 mmol) was combined in DCM (2.0 ml) with aniline (0.022 ml, 0.242 mmol), EDAC (69.6 mg, 0.363 mmol) and DMAP (2 mg). The resulting mixture was stirred at room temperature for 16 h, loaded on preparative TLC (silica) and developed with 7% MeOH in DCM to give 32.5 mg (51%) of the title product.

ESI-MS calc. for C33H33N3O: 487; Found: 488 (M+H).

### Example 54

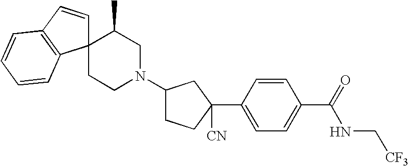

The amino acid from Step E of Example 52 (50 mg, 0.121 mmol) was combined in DCM (2.0 ml) with trifluoroethylamine hydrochloride (32.8 ml, 0.242 mmol), EDAC (69.6 mg, 0.363 mmol) and DMAP (2 mg). The resulting mixture was stirred at room temperature for 16 h, loaded on preparative TLC (silica) and developed with 7% MeOH in DCM to give 36.3 mg (57%) of the title product.

ESI-MS calc. for C29H30F3N3O: 493; Found: 494 (M+H).

### Example 55

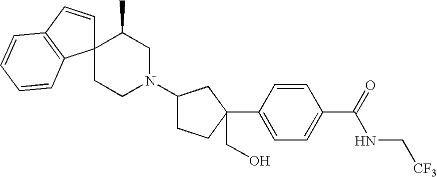

### Step A:

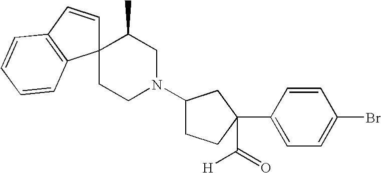

To the stirred solution of the phenylbromide (1.185 g, 2.65 mmol) from Step C of Example 52 in ether (40 ml) at 0° C. was added a solution of DIBAL (1.0 M, 4 ml, 4 mmol) in toluene dropwise under nitrogen atmosphere. The mixture was stirred at 0° C. for 1 h. TLC showed a complete conversion. The reaction was quenched by adding MeOH (10 ml), filtered through silica gel in a frit funnel, washed with 20% MeOH in DCM, concentrated in vacuo to afford 0.971 g (81%) of the title product as white foam.

ESI-MS calc. for C26H28BrNO: 449; Found: 450 (M+H).

### Step B:

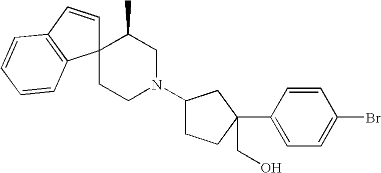

The aldehyde (0.96 g, 2.13 mmol) from Step A immediately above was taken up in MeOH (30 ml), sodium borohydride (0.563 g, 15 mmol) was added at RT with stirring. The starting aldehyde was not completely soluble in MeOH, ˜5 ml of THF was added. The resulting transparent solution was stirred for 1 h, TLC showed a complete conversion. The reaction was quenched by adding water (5 ml), evaporated to dryness. The residue was diluted with water (10 ml), extracted with DCM (4×20 ml). The combined organic phases were dried over sodium sulfate, filtered and evaporated to dryness. The residue was purified on FC (silica gel, 10% MeOH in DCM) to give 469.8 mg of the title product.

ESI-MS calc. for C26H30BrNO: 451; Found: 450 (M+H).

### Step C:

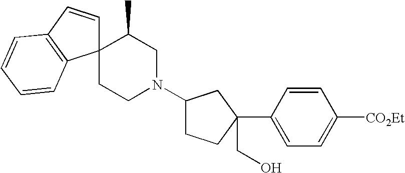

The phenylbromide from Step B immediately above (460 mg, 1.017 mmol) was combined in a mixture of triethylamine (124 mg, 1.22 mmol), palladium chloride (36 mg, 0.203 mmol), triphenylphosphine (107 mg, 0.406 mmol) in ethanol (15 ml). The mixture vacuum and then flushed with carbon monoxide before heating started. The procedure was repeated three times and the mixture was then stirred under atmosphere of carbon monoxide at 100° C. for 3 days. TLC showed the reaction was messy. After filtered off the solid catalyst, the solution was evaporated to dryness. The residue was purified on preparative TLC (silica gel, 10% MeOH/DCM) to afford 127.6 mg of the title product which was contaminated with de-bromated starting material.

ESI-MS calc. for C29H35NO3: 445; Found: 446 (M+H).

### Step D:

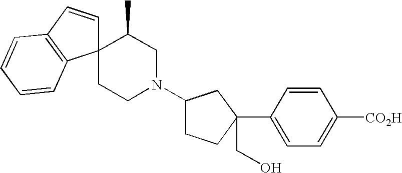

The crude ester from Step C immediately above (127 mg, 0.28 mmol) was combined in a mixture of dioxane (4 ml) and water (2 ml) with lithium hydroxide monohydrate (42 mg, 1.0 mmol). The resulting mixture was stirred at RT for 4 h. The reaction mixture was condensed to dryness and purified on preparative TLC (silica, 25% methanol/DCM) to give the polar 12.5 mg of the title product.

ESI-MS calc. for C27H31NO3: 417; Found: 418 (M+H).

### Step E:

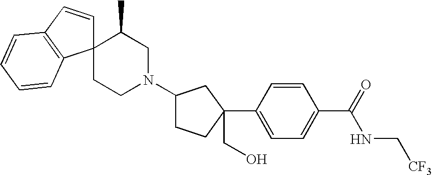

The amino acid from Step D immediately above (11 mg, 0.0263 mmol) was combined in DCM (1 ml) with trifluoroethylamine hydrochloride (10.7 mg, 0.0789 mmol), EDAC (20.2 mg, 0.1052 mmol) and triethylamine (0.013 ml, 0.0789 mmol). The resulting mixture was stirred at room temperature for 16 h, condensed and loaded on preparative TLC (silica), developed with 10% methanol/DCM to give 7.8 mg (55%) of the title product.

ESI-MS calc. for C29H33F3N2O2: 498; Found: 499 (M+H).

### Example 56

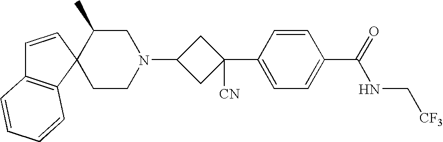

### Step A:

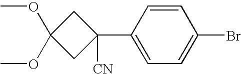

To a cool (0° C.) solution of 4-bromophenylacetonitrile (9.8 g, 50 mmol) in DMF (200 ml) was added sodium hydride (60% oil, 4.8 g, 120 mmol) in multiple portions under nitrogen protection. Stirring was continued until the cease of bubble formation, then 1,3-dibromo-2,2-dimethoxypropane (13.0 g, 50 mmol) was added in one portion. The reaction was stirred at RT overnight, then at 65° C. for 3 h, cooled at RT, quenched with ice-water (500 ml), extracted with ether (3×500 ml). The combined ether layers were washed with water (2×500 ml), dried over sodium sulfate, filtered, evaporated. The crude brown-dark residue was used for further hydrolysis without purification.

### Step B:

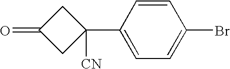

The entire crude product from Step A immediately above was stirred with a mixture of TFA (25 ml) and DCM (25 ml) at RT overnight, evaporated to dryness. The residue was purified on FC (silica gel, 10% ethyl acetate/hexane) to afford 4.2 g of the title product as light brown oil. ¹H-NMR (CDCl₃, 300 MHz): δ 7.58 (d, J=8.76 Hz, 2H), 7.38 (d, J=8.76 Hz, 1H), 4.09 (d, J=2.41 Hz, 2H), 3.70 (d, J=2.41 Hz, 2H).

### Step C:

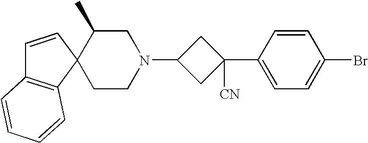

The cyclobutanone from Step B immediately above (3.152 g, 12.6 mmol) was combined in DCM (70 mL) with 3-methylspiroindenepiperidine Intermediate 1 (2.971 g, 12.6 mmol), DMA (2.195 ml, 22.8 mmol), sodium triacetoxyborohydride (5.342 g, 25.2 mmol), and molecular sieves (4A, 5.0 g). The resulting mixture was stirred at room temperature for 24 h. The reaction mixture was then filtered through a celite plug, washing with 50% methanol in DCM. The filtrates were concentrated and purified on FC (silica, 40% ethyl acetate in hexane) to give 5.447 g of the title product.

ESI-MS calc. for C25H25BrN2: 433; Found: 434 (M+H).

### Step D:

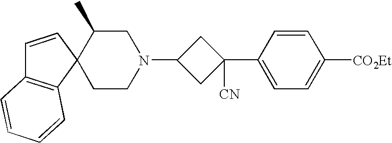

The phenylbromide from Step C immediately above (5.4 g, 12.5 mmol) was combined in a mixture of triethylamine (2.1 ml 15 mmol), palladium chloride (221 mg, 1.25 mmol), triphenylphosphine (656 mg, 2.5 mmol) in ethanol (50 ml). The mixture vacuum and then flushed with carbon monoxide. The procedure was repeated three times and the mixture was then stirred under atmosphere of carbon monoxide at 100° C. for 3 days. After filtered off the solid catalyst, the solution was evaporated to dryness. The residue was purified on preparative TLC (silica gel, 10% MeOH/DCM) to collect all the possible products as a mixture which was used in next step without further purification.

### Step E:

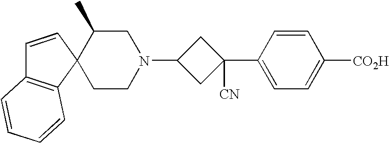

The entire material from Step D immediately above was combined in a mixture of dioxane (14 ml) and water (7 ml) with lithium hydroxide monohydrate (210 mg, 5 mmol). The resulting mixture was stirred at RT for 4 h. The reaction mixture was condensed to dryness and purified on preparative TLC (silica, 10% methanol/DCM) to give 1.28 g of the title product which was contaminated by other impurities.

ESI-MS calc. for C26H26N2O2: 398; Found: 399 (M+H).

### Step F:

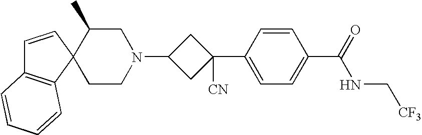

The amino acid from Step E immediately above (199 mg, 0.5 mmol) was combined in DCM (3 ml) with trifluoroethylamine hydrochloride (135.5 mg, 1.0 mmol), EDAC (383.4 mg, 2.0 mmol) and DMAP (5 mg). The resulting mixture was stirred at room temperature for 16 h, condensed and loaded on preparative TLC (silica), developed with 40% ethyl acetate/hexane to give 9.0 mg of the title product.

ESI-MS calc. for C28H28F3N3O: 479; Found: 480 (M+H).

### Example 57

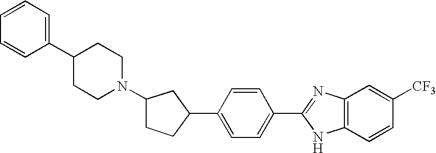

### Step A:

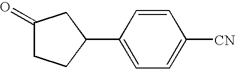

A mixture of cyclopentenone (10.0 g, 120 mmol), 4-cyanobenzene boric acid (14.6 g, 100 mmol), sodium acetate (16.4 g, 200 mmol), palladium acetate (4.60 g, 20 mmol), antimony trichloride (4.60 g, 20 mmol) in acetic acid (500 ml) was stirred over two days. The dark solid was removed by filtration and the filtrate was evaporated to remove acetic acid under reduced pressure. To the residue was added water (200 mL) and ethyl acetate (400 ml), stirred for 30 min. The organic phase was separated and washed with brine (200 ml), dried over anhydrous sodium sulfate, filtered and evaporated. The residue was chromatographed on silica gel (eluted with 20% ethyl acetate in hexane) to afford 7.5 g of the title compound as a yellow oil. ¹H-NMR (CDCl₃, 300 MHz): δ 7.62 (d, J=8.62 Hz, 2H), 7.38 (d, J=8.62 Hz, 1H), 3.35 (m, 1H), 2.20-2.80 (m, 5H), 2.00 (m, 1H).

### Step B:

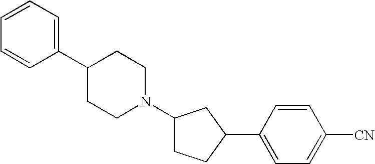

The ketone from Step A immediately above (1.798 g, 9.7 mmol) was combined in DCM (50 mL) with 4-phenylpiperidine hydrochloride (2.303 g, 11.64 mmol), DIEA (2.03 ml, 11.64 mmol), sodium triacetoxyborohydride (6.17 g, 29.1 mmol), and molecular sieves (4A, 5.0 g). The resulting mixture was stirred at room temperature for 24 h. The reaction mixture was then filtered through a celite plug, washing with ethyl acetate. The filtrate was washed with saturated NaHCO₃ solution, then with brine, dried over anhydrous MgSO₄, filtered, and concentrated to give the crude product which was used in next step without purification.

ESI-MS calc. for C23H26N2: 330; Found: 331 (M+H).

### Step C:

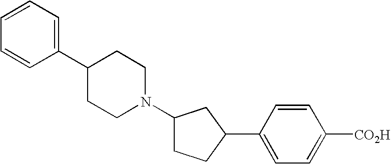

The entire material (˜9.7 mmol) from Step B immediately above was combined in a mixture of ethanol (20 ml) and water (10 ml) with sodium hydroxide (1.94 g, 48.5 mmol). The resulting mixture was stirred at reflux for 4 h. TLC showed a complete conversion. The reaction was neutralized with 3N aq. HCl (˜33 ml) until pH=7-8. This aqueous mixture was extracted with DCM (5×100 ml). The combined organic phases were condensed to dryness. The resulting solid residue was dissolved in methanol/DCM (1:1) and loaded on a FC column (silica gel), eluted with 10% MeOH in DCM to give 3.24 g (96% in two steps) of the title product.

ESI-MS calc. for C23H27NO2: 349; Found: 350 (M+H).

### Step D:

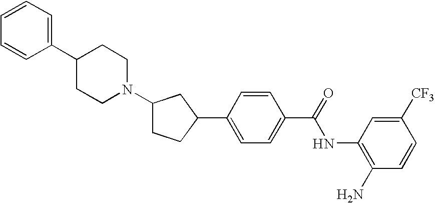

The amino acid from Step C immediately above (100 mg, 0.286 mmol) was combined in DCM (2 ml) with 4-trifluoromethyl-1,2-phenylenediamine (151.4 mg, 0.858 mmol), EDAC (219.3 mg, 1.144 mmol) and DMAP (7 mg). The resulting mixture was stirred at room temperature for 16 h, condensed and loaded on preparative TLC (silica), developed with ethyl acetate to give 123.4 mg (85%) of the title product.

ESI-MS calc. for C30H32F3N3O: 507; Found: 508 (M+H).

### Step E:

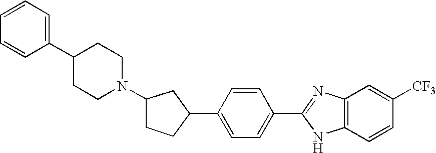

The aminoamide from Step D immediately above (123 mg, 0.243) in 2 ml of acetic acid was heated at 60° C. overnight. The acetic acid was removed under reduced pressure and the residue was purified on preparative TLC (silica, 1/9/90 of NH₄OH/methanol/DCM) to give 46 mg (34%) of the title product aminoimidazole as a mixture of 4 diastereomers.

ESI-MS calc. for C30H30F3N3: 489; Found: 450 (M+H).

### Example 58

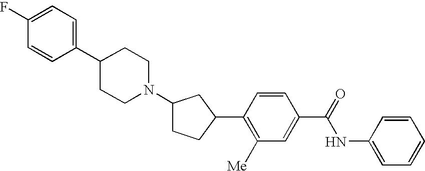

### Step A:

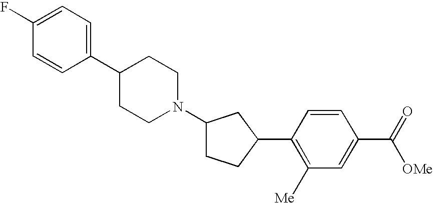

The cyclopentanone from Step A of Example 15 (100 mg, 0.430 mmol) was combined in DCM (5 mL) with 4-(para-fluorophenyl)piperidine hydrochloride (111.3 mg, 0.516 mmol), DIEA (0.090 ml, 0.516 mmol), sodium triacetoxyborohydride (364.6 mg, 1.72 mmol), and molecular sieves (4A, 0.50 g). The resulting mixture was stirred at room temperature for 24 h. The reaction mixture was then filtered through a celite plug, washing with methanol. The filtrates were concentrated and purified on preparative TLC (silica, 10% MeOH in DCM) to give 124 mg (73%) of the title product.

ESI-MS calc. for C25H30FNO2: 395; Found: 396 (M+H).

### Step B:

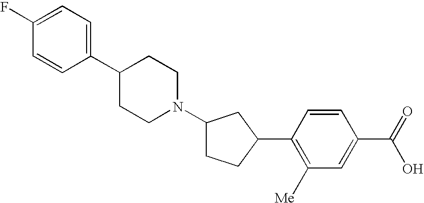

The ester from Step A immediately above (110 mg, 0.278 mmol) was combined in a mixture of ethanol (4 ml) and water (2 ml) with lithium hydroxide monohydrate (70.1 mg, 1.668 mmol). The resulting mixture was stirred at RT overnight. The reaction mixture was condensed to dryness and purified on preparative TLC (silica, 20% methanol/DCM) to give 92.1 mg (87%) of the title product amino acid.

ESI-MS calc. for C24H28FNO2: 381; Found: 382 (M+H).

### Step C:

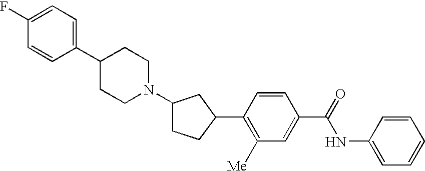

The amino acid from Step B immediately above (70 mg, 0.184 mmol) was combined in DCM (2 ml) with aniline (0.05 ml, 0.552 mmol), EDAC (176.4 mg, 0.920 mmol) and DMAP (4.5 mg). The resulting mixture was stirred at room temperature for 16 h, condensed and loaded on preparative TLC (silica), developed with 10% MeOH/DCM to give 63.2 mg (70%) of the title product.

ESI-MS calc. for C30H33FN20: 456; Found: 457 (M+H).

### Example 59

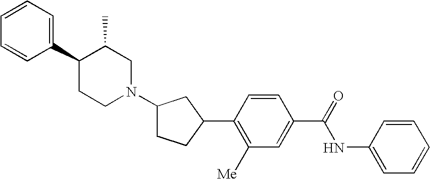

### Step A:

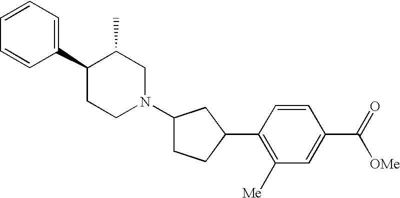

The cyclopentanone from Step A of Example 15 (100 mg, 0.430 mmol) was combined in DCM (5 mL) with racemic trans-3-methyl-4-phenylpiperidine hydrochloride (109.2 mg, 0.516 mmol), DIEA (0.090 ml, 0.516 mmol), sodium triacetoxyborohydride (364.6 mg, 1.72 mmol), and molecular sieves (4A, 0.50 g). The resulting mixture was stirred at room temperature for 24 h. The reaction mixture was then filtered through a celite plug, washing with methanol. The filtrates were concentrated and purified on preparative TLC (silica, 10% MeOH in DCM) to give 151.4 mg (90%) of the title product.

ESI-MS calc. for C26H33NO2: 391; Found: 392 (M+H).

### Step B:

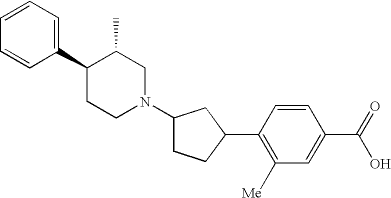

The ester from Step A immediately above (145 mg, 0.370 mmol) was combined in a mixture of ethanol (4 ml) and water (2 ml) with lithium hydroxide monohydrate (93.4 mg, 2.22 mmol). The resulting mixture was stirred at RT overnight. The reaction mixture was condensed to dryness and purified on preparative TLC (silica, 20% methanol/DCM) to give 52.4 mg (38%) of the title product amino acid.

ESI-MS calc. for C25H31NO2: 377; Found: 378 (M+H).

### Step C:

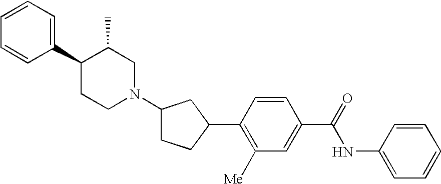

The amino acid from Step B immediately above (45 mg, 0.119 mmol) was combined in DCM (2 ml) with aniline (0.033 ml, 0.357 mmol), EDAC (114.1 mg, 0.595 mmol) and DMAP (3.0 mg). The resulting mixture was stirred at room temperature for 16 h, condensed and loaded on preparative TLC (silica), developed with 10% MeOH/DCM to give 46.8 mg (80%) of the title product.

ESI-MS calc. for C31H36N2O: 452; Found: 453 (M+H).

### Example 60

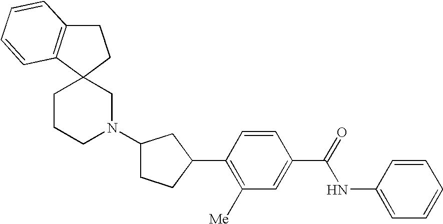

### Step A:

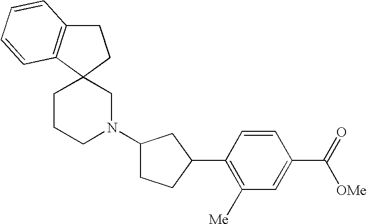

The cyclopentanone from Step A of Example 15 (100 mg, 0.430 mmol) was combined in DCM (5 mL) with racemic 3-spiroindanepiperidine hydrochloride (115.5 mg, 0.516 mmol), DIEA (0.090 ml, 0.516 mmol), sodium triacetoxyborohydride (364.6 mg, 1.72 mmol), and molecular sieves (4A, 0.50 g). The resulting mixture was stirred at room temperature for 24 h. The reaction mixture was then filtered through a celite plug, washing with methanol. The filtrates were concentrated and purified on preparative TLC (silica, 10% MeOH in DCM) to give 163.1 mg (94%) of the title product.

ESI-MS calc. for C27H33NO2: 403; Found: 404 (M+H).

### Step B:

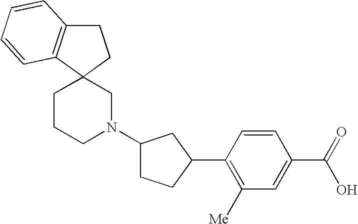

The ester from Step A immediately above (150 mg, 0.372 mmol) was combined in a mixture of ethanol (4 ml) and water (2 ml) with lithium hydroxide monohydrate (94 mg, 2.22 mmol). The resulting mixture was stirred at RT overnight. The reaction mixture was condensed to dryness and purified on preparative TLC (silica, 20% methanol/DCM) to give 68.5 mg (47%) of the title product amino acid.

ESI-MS calc. for C26H31NO2: 389; Found: 390 (M+H).

### Step C:

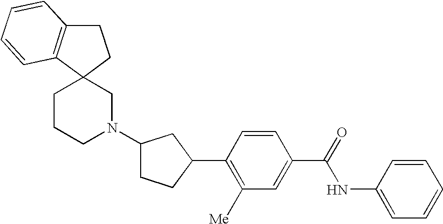

The amino acid from Step B immediately above (60 mg, 0.154 mmol) was combined in DCM (2 ml) with aniline (0.042 ml, 0.462 mmol), EDAC (148 mg, 0.770 mmol) and DMAP (4.0 mg). The resulting mixture was stirred at room temperature for 16 h, condensed and loaded on preparative TLC (silica), developed with 10% MeOH/DCM to give 63.2 mg (82%) of the title product.

ESI-MS calc. for C32H36N2O: 464; Found: 465 (M+H).

### Example 61

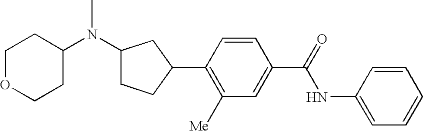

### Step A:

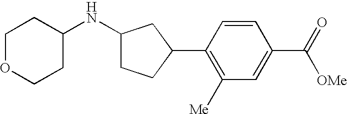

The cyclopentanone from Step A of Example 15 (1.0 g, 4.3 mmol) was combined in DCM (50 mL) with tetrahydropyranylamine hydrochloride (887 mg, 6.45 mmol), DMA (1.15 ml), sodium triacetoxyborohydride (5.47 g, 25.8 mmol), and molecular sieves (4A, 5.0 g). The resulting mixture was stirred at room temperature for 24 h. The reaction mixture was then filtered through a celite plug, washing with methanol. The filtrates were concentrated and purified on preparative TLC (silica, 10% MeOH in DCM) to give 1.296 g (95%) of the title product.

ESI-MS calc. for C19H27NO3: 317; Found: 318 (M+H).

### Step B:

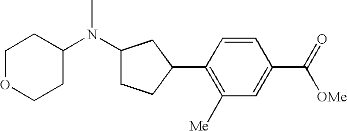

The free amino ester from Step A immediately above (150 mg, 0.473 mmol) was combined in DCM (5 mL) with 37% formaldehyde in water (382 mg, 4.73 mmol) and molecular sieves (4A, 1.0 g). The resulting mixture was stirred for 15 min, then sodium triacetoxyborohydride (1.0 g, 4.73 mmol) was added. The resulting mixture was stirred at room temperature overnight. The reaction mixture was then filtered through a celite plug, washing with methanol. The filtrates were concentrated and purified on preparative TLC (silica, 10% MeOH in DCM) to give 148.7 mg (95%) of the title product.

ESI-MS calc. for C20H29NO3: 331; Found: 332 (M+H).

### Step C:

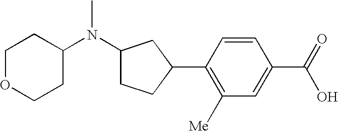

The amino ester from Step B immediately above (140 mg, 0.423 mmol) was combined in a mixture of ethanol (3 ml) and water (1.5 ml) with lithium hydroxide monohydrate (106.5 mg, 2.538 mmol). The resulting mixture was stirred at RT overnight. The reaction mixture was condensed to dryness and purified on preparative TLC (silica, 50% methanol/DCM) to give 103.1 mg (77%) of the title product amino acid.

ESI-MS calc. for C19H27NO3: 317; Found: 317 (M+H).

### Step D:

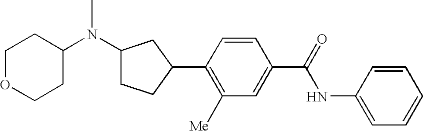

The amino acid from Step C immediately above (50 mg, 0.157 mmol) was combined in DCM (1 ml) with aniline (0.043 ml, 0.462 mmol), EDAC (150 mg, 0.785 mmol) and DMAP (4.0 mg). The resulting mixture was stirred at room temperature for 16 h, condensed and loaded on preparative TLC (silica), developed with 10% MeOH/DCM to give 47.8 mg (70%) of the title product.

ESI-MS calc. for C25H32N2O2: 392; Found: 393 (M+H).

### Example 62

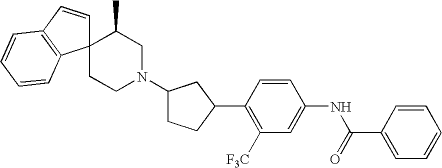

### Step A:

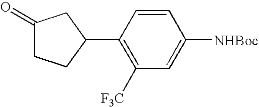

To a thick wall pressure tube was added N-Boc-4-bromo-3-trifluoroaniline (7.06 g, 20.83 mmol), cyclopentenone (8.75 ml, 104.15 mmol), triethyl amine (4.355 ml, 32.7 mmol), palladium acetate (93.5 mg, 0.417 mmol) and triphenyl phosphine (218.7 mg, 0.834). The tube was capped and stirred in 100° C. oil bath for 3 days. TLC showed the reaction was still not complete. The entire mixture was loaded on silica gel column without any workup, eluted with 30% ethyl acetate in hexane to afford 1.32 g (18%) of the title compound (second major spot on TLC). ¹H-NMR (CDCl₃, 300 MHz): δ 7.68 (m, 1H), 7.55 (m, 1H), 7.35 (m, 1H), 7.08 (ms, 1H), 3.70 (m, 1H), 2.20-2.70 (m, 5H), 2.00 (m, 1H), 1.48 (m, 9H).

### Step B:

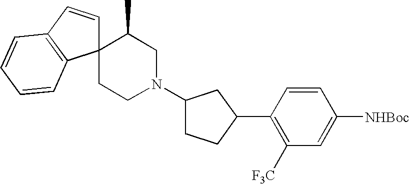

The cyclopentanone from Step A immediately above (0.82 g, 2.39 mmol) was combined in DCM (50 mL) with 3-methylspiroindenepiperidine Intermediate 1 (0.676 g, 2.868 mmol), DIEA (0.5 ml, 2.868 mmol), sodium tiacetoxyborohydride (2.027 g, 12.9 mmol), and molecular sieves (4A, 5.0 g). The resulting mixture was stirred at room temperature for 24 h. The reaction mixture was then filtered through a celite plug, washing with methanol. The filtrates were concentrated and purified on FC (silica, 80% ethyl acetate in hexane) to give 1.257 g (99%) of the title product.

ESI-MS calc. for C31H37F3N2O2: 526; Found: 527 (M+H).

### Step C:

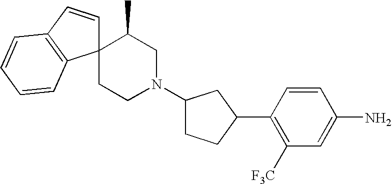

The carbamide from Step B immediately above (1.157 g, 2.20 mmol) was dissolved in a neat TFA (15 ml), stirred at RT for 30 min, evaporated to dryness. The residue was dissolved in DCM (50 ml), washed with aq. sodium bicarbonate (3×50 ml), dried over sodium sulfate, evaporated to dryness to give 0.77 g (82%) of the title product as white foam.

ESI-MS calc. for C26H29F3N2: 426; Found: 427 (M+H).

### Step D:

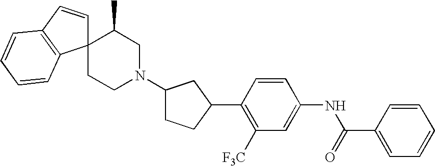

The aniline from Step C immediately above (100 mg, 0.234 mmol) was combined in DCM (3 ml) with benzoic acid (57.2 mg, 0.468 mmol), EDAC (179.4 mg, 0.936 mmol) and DMAP (6 mg). The resulting mixture was stirred at room temperature for 16 h, condensed and loaded on preparative TLC (silica), developed with 10% MeOH/DCM to give 121.7 mg (92%) of the title product.

ESI-MS calc. for C33H33F3N2O: 530; Found: 531 (M+H).

### Example 63

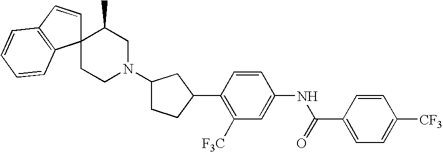

The aniline from Step C of Example 62 (50 mg, 0.117 mmol) was combined in DCM (2 ml) with 4-trifluoromethylbenzoic acid (89 mg, 0.468 mmol), EDAC (179.4 mg, 0.936 mmol) and DMAP (6 mg). The resulting mixture was stirred at room temperature for 16 h, condensed and loaded on preparative TLC (silica), developed with 8% MeOH/DCM to give 78.6 mg (100%) of the title product.

ESI-MS calc. for C34H32F6N2O: 598; Found: 599 (M+H).

### Example 64

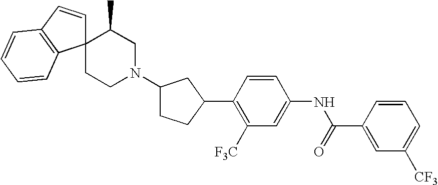

The aniline from Step C of Example 62 (50 mg, 0.117 mmol) was combined in DCM (2 ml) with 3-trifluoromethylbenzoic acid (89 mg, 0.468 mmol), EDAC (179.4 mg, 0.936 mmol) and DMAP (6 mg). The resulting mixture was stirred at room temperature for 16 h, condensed and loaded on preparative TLC (silica), developed with 8% MeOH/DCM to give 76.7 mg (99%) of the title product.

ESI-MS calc. for C34H32F6N2O: 598; Found: 599 (M+H).

### Example 65

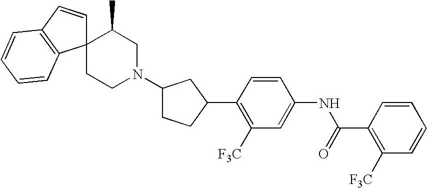

The aniline from Step C of Example 62 (50 mg, 0.117 mmol) was combined in DCM (2 ml) with 2-trifluoromethylbenzoic acid (89 mg, 0.468 mmol), EDAC (179.4 mg, 0.936 mmol) and DMAP (6 mg). The resulting mixture was stirred at room temperature for 16 h, condensed and loaded on preparative TLC (silica), developed with 8% MeOH/DCM to give 73.3 mg (98%) of the title product.

ESI-MS calc. for C34H32F6N2O: 598; Found: 599 (M+H).

### Example 66

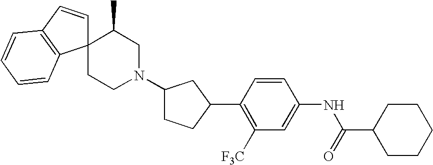

The aniline from Step C of Example 62 (50 mg, 0.117 mmol) was combined in DCM (2 ml) with cyclohexane carboxylic acid (60 mg, 0.468 mmol), EDAC (179.4 mg, 0.936 mmol) and DMAP (6 mg). The resulting mixture was stirred at room temperature for 16 h, condensed and loaded on preparative TLC (silica), developed with 8% MeOH/DCM to give 59 mg (88%) of the title product.

ESI-MS calc. for C33H39F3N2O: 536; Found: 537 (M+H).

### Example 67

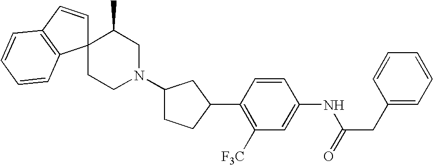

The aniline from Step C of Example 62 (50 mg, 0.117 mmol) was combined in DCM (2 ml) with phenyl acetic acid (63.7 mg, 0.468 mmol), EDAC (179.4 mg, 0.936 mmol) and DMAP (6 mg). The resulting mixture was stirred at room temperature for 16 h, condensed and loaded on preparative TLC (silica), developed with 10% MeOH/DCM to give 70.1 mg (100%) of the title product.

ESI-MS calc. for C34H35F3N2O: 544; Found: 545 (M+H).

### Example 68

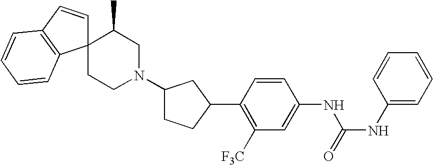

The aniline from Step C of Example 62 (50 mg, 0.117 mmol) was combined in DCM (2 ml) with phenyl isocyante (0.0636 ml, 0.585 mmol). The resulting mixture was stirred at room temperature for 16 h, condensed and loaded on preparative TLC (silica), developed with 10% MeOH/DCM to give 52.8 mg (78%) of the title product.

ESI-MS calc. for C33H34F3N3O: 545; Found: 546 (M+H).

### Example 69

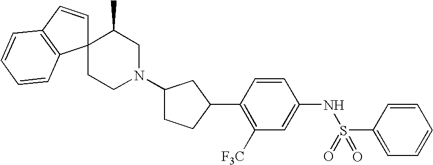

The aniline from Step C of Example 62 (50 mg, 0.117 mmol) was combined in DCM (2 ml) with benzene sulfonyl chloride (0.018 ml, 0.234 mmol). The resulting mixture was stirred at room temperature for 16 h, condensed and loaded on preparative TLC (silica), developed with 10% MeOH/DCM to give 50.0 mg (71%) of the title product.

ESI-MS calc. for C32H32F3N2O2S: 566; Found: 567 (M+H).

### Example 70

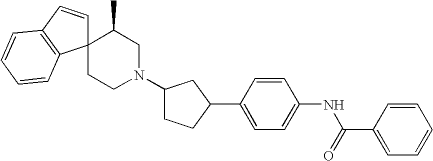

### Step A

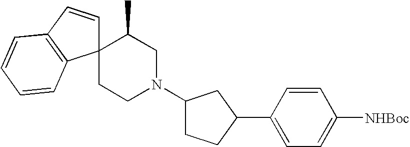

The amino acid from Step D of Example 1 (350 mg, 0.903 mmol) was combined in toluene (3 ml) with TEA (109.6 mg, 1.086 mmol) and diphenyl phosphoryl azide (0.214 ml, 1.0 mmol). The resulting mixture was stirred at 90° C. for 2 h, and then tert-butanol (4 ml) was added. The mixture was stirred at 90° C. overnight, condensed and loaded on preparative TLC (silica), developed with 10% MeOH/DCM to give 82.8 mg of the title product.

ESI-MS calc. for C30H38N2O2: 458; Found: 459 (M+H).

### Step B

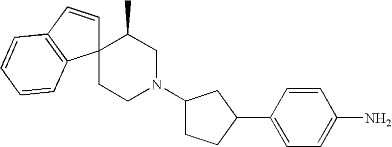

The carbamide from Step A immediately above (82.8 mg) was dissolved in TFA (2 ml). The resulting mixture was stirred at RT for 30 min, condensed and loaded on preparative TLC (silica), developed with 1%:9%:90% of aq. NH4OH/MeOH/DCM to give 53.5 mg (91%) of the title product.

ESI-MS calc. for C25H30N2: 358; Found: 359 (M+H).

### Step C

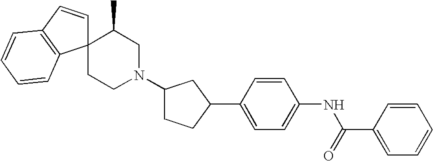

The aniline from Step B immediately above (23 mg, 0.064 mmol) was combined in DCM (1 ml) with benzoic acid (15.7 mg, 0.128 mmol), EDAC (37 mg, 0.192 mmol) and DMAP (2 mg). The resulting mixture was stirred at room temperature for 2 days, condensed and loaded on preparative TLC (silica), developed with 10% MeOH/DCM to give 25.4 mg (77%) of the title product.

ESI-MS calc. for C32H34N2O: 477; Found: 478 (M+H).

### Example 71

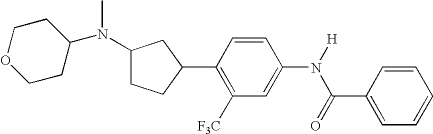

### Step A:

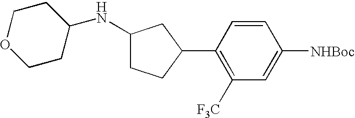

The cyclopentanone from Step A of Example 62 (150 mg, 0.437 mmol) was combined in DCM (5 mL) with tetrahydropyranylamine hydrochloride (90.1 mg, 0.655 mmol), DIEA (0.152 ml, 0.874 mmol), sodium triacetoxyborohydride (371 mg, 1.748 mmol), and molecular sieves (4A, 1.0 g). The resulting mixture was stirred at room temperature for 24 h. The reaction mixture was then filtered through a celite plug, washing with methanol. The filtrates were concentrated and purified on preparative TLC (silica, 10% MeOH in DCM) to give 204.2 mg (100%) of the title product.

ESI-MS calc. for C22H31F3N2O3: 428; Found: 428 (M+H).

### Step B:

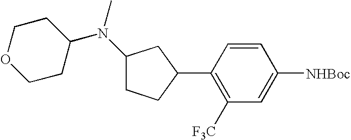

The amine from Step A immediately above (150 mg, 0.350 mmol) was combined in DCM (5 mL) with 37% formaldehyde in water (283 mg, 3.5 mmol) and molecular sieves (4A, 2.0 g). The resulting mixture was stirred for 15 min, then sodium triacetoxyborohydride (742 mg, 3.5 mmol) was added. The resulting mixture was stirred at room temperature overnight. The reaction mixture was then filtered through a celite plug, washing with methanol. The filtrates were concentrated and purified on preparative TLC (silica, 10% MeOH in DCM) to give 125.3 mg of the title product.

ESI-MS calc. for C23H33F3N2O3: 442; Found: 443 (M+H).

### Step C:

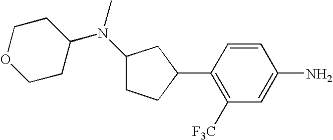

The amino ester from Step B immediately above (100 mg, 0.226 mmol) was dissolved in neat TFA (2 ml). The resulting mixture was stirred at RT for 30 min. The reaction mixture was condensed to dryness, dissolved in DCM (20 ml), washed with aq. sodium bicarbonate, dried over sodium sulfate and evaporated to dryness to give 75 mg (97%) of the title product.

ESI-MS calc. for C18H25F3N2O: 342; Found: 343 (M+H).

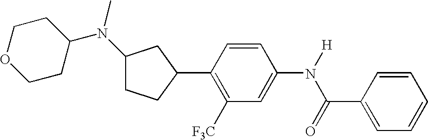

### Step D:

The aniline from Step C immediately above (50 mg, 0.146 mmol) was combined in DCM (2 ml) with benzoic acid (71.3 mg, 0.584 mmol), EDAC (448 mg, 2.346 mmol) and DMAP (7.0 mg). The resulting mixture was stirred at room temperature for 16 h, condensed and loaded on preparative TLC (silica), developed with 1:9:90% aq. NH₄OH/MeOH/DCM to give 72 mg of the title product.

ESI-MS calc. for C25H29F3N2O2: 446; Found: 447 (M+H).

### Example 72

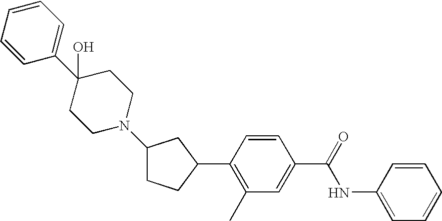

### Step A:

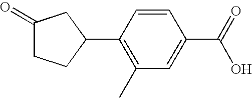

The ester from Step A of Example 15 (1.0 g, 4.31 mmol) was combined in a mixture of ethanol (10 ml) and water (5 ml) with lithium hydroxide monohydrate (0.362 g, 8.62 mmol). The resulting mixture was stirred at RT overnight. The reaction mixture was condensed to dryness, the residue was partitioned between ethyl acetate (50 ml) and 1N HCl aqueous solution (50 ml), separated. The aq. solution was extracted with ethyl acetate (2×50 ml). The combined organic layers were dried over sodium sulfate, evaporated to dryness to give 850 mg (90%) of the title product as brown foam.

ESI-MS calc. for C13H14O3: 218; Found: 219 (M+H).

### Step B:

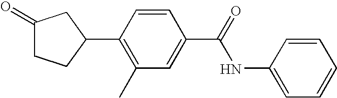

The acid from Step A immediately above (800 mg, 3.67 mmol) was combined in DCM (30 ml) with aniline (334.4 mg, 3.67 mmol), EDAC (1.41 g, 7.34 mmol) and DMAP (22 mg). The resulting mixture was stirred at room temperature for 2 days, diluted with DCM (150 ml) and washed with aq. 1N HCl (3×50 ml). The organic layers were dried over sodium sulfate and evaporated to dryness to give 1.01 g of the title product with good purity.

ESI-MS calc. for C19H19NO2: 293; Found: 294 (M+H).

### Step C:

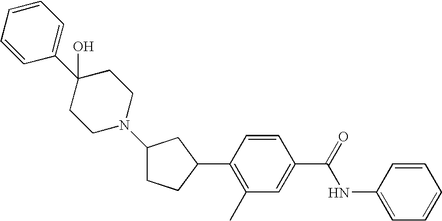

The cyclopentanone from Step B immediately above (100 mg, 0.341 mmol) was combined in DCM (3 mL) with 4-hydroxy-4-phenylpiperidine (90.7 mg, 0.5115 mmol), sodium triacetoxyborohydride (289 mg, 1.364 mmol), and molecular sieves (4A, 500 mg). The resulting mixture was stirred at room temperature overnight. The reaction mixture was then filtered through a celite plug, washing with methanol. The filtrates were concentrated and purified on preparative (silica, 10% MeOH in DCM) to give 17 mg (11%) of the title product.

ESI-MS calc. for C30H34N2O2: 454; Found: 455 (M+H).

### Example 73

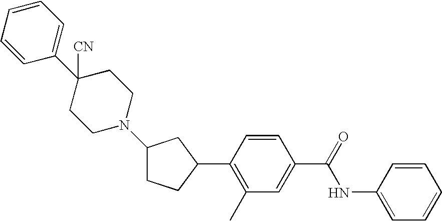

The cyclopentanone from Step B of Example 72 (100 mg, 0.341 mmol) was combined in DCM (3 mL) with 4-cyano-4-phenylpiperidine hydrochloride (114 mg, 0.5115 mmol), DIEA (0.089 ml, 0.5115 mmol), sodium triacetoxyborohydride (289 mg, 1.364 mmol), and molecular sieves (4A, 500 mg). The resulting mixture was stirred at room temperature overnight. The reaction mixture was then filtered through a celite plug, washing with methanol. The filtrates were concentrated and purified on preparative (silica, 10% MeOH in DCM) to give 20.5 mg (12%) of the title product.

ESI-MS calc. for C31H33N3O: 463; Found: 463 (M+H).

### Example 74

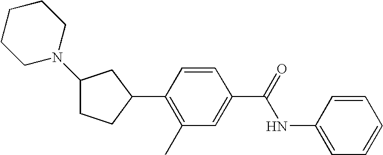

The cyclopentanone from Step B of Example 72 (100 mg, 0.341 mmol) was combined in DCM (3 mL) with piperidine (0.051, 0.5115 mmol), sodium triacetoxyborohydride (289 mg, 1.364 mmol), and molecular sieves (4A, 500 mg). The resulting mixture was stirred at room temperature overnight. The reaction mixture was then filtered through a celite plug, washing with methanol. The filtrates were concentrated and purified on preparative (silica, 10% MeOH in DCM) to give 26 mg (19%) of the title product.

ESI-MS calc. for C24H30N2O: 362; Found: 363 (M+H).

### Example 75

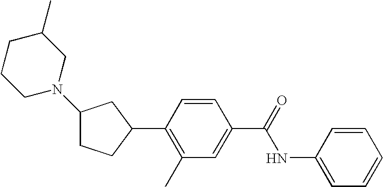

The cyclopentanone from Step B of Example 72 (100 mg, 0.341 mmol) was combined in DCM (3 mL) with 3-methylpiperidine (0.060, 0.5115 mmol), sodium triacetoxyborohydride (289 mg, 1.364 mmol), and molecular sieves (4A, 500 mg). The resulting mixture was stirred at room temperature overnight. The reaction mixture was then filtered through a celite plug, washing with methanol. The filtrates were concentrated and purified on preparative (silica, 10% MeOH in DCM) to give 19.8 mg (14%) of the title product.

ESI-MS calc. for C25H32N2O: 376; Found: 377 (M+H).

### Example 76

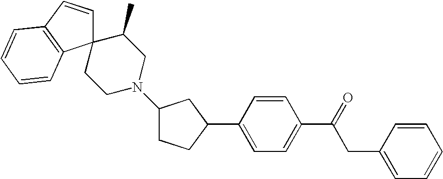

### Step A

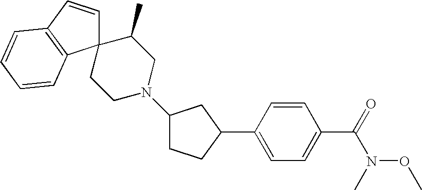

The amino acid from Step D of Example 1 (387 mg, 1 mmol) was combined in DCM (5.0 ml) with N,O-dimethylhydroxylamine hydrochloride (200 mg, 2.0 mmol), EDAC (382 mg, 2.0 mmol). The resulting mixture was stirred at room temperature for 16 h, loaded on preparative TLC (silica) and developed with 10% MeOH in DCM to give 274 mg of the title product as a light brown solid.

ESI-MS calc. for C28H34N2O2: 430; Found: 431 (M+H).

### Step B

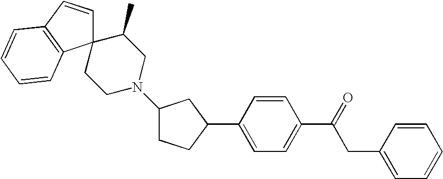

The aminoamide from Step A immediately above (96 mg, 0.2) in 2.0 ml of THF was treated with benzylmagnesium chloride in THF (2.0 M, 2.0 ml, 4.0 mmol) at RT for 2 h. The entire mixture was loaded on preparative TLC (silica gel) and developed with 5% MeOH in DCM to give 72 mg of the title product as a mixture of 4 diastereomers.

ESI-MS calc. for C33H35NO: 461; Found: 462 (M+H).

### Example 77

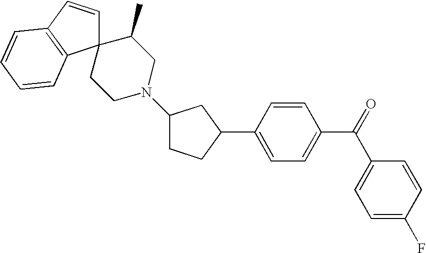

The aminoamide from Step A of Example 75 (96 mg, 0.2) in 2.0 ml of THF was treated with 4-fluorophenylmagnesium bromide (1.0 M, 4.0 ml, 4.0 mmol) at RT for 2 h. The entire mixture was loaded on preparative TLC (silica gel) and developed with 5% MeOH in DCM to give 54 mg of the title product as a mixture of 4 diastereomers.

ESI-MS calc. for C32H32FNO: 465; Found: 466 (M+H).

### Example 78

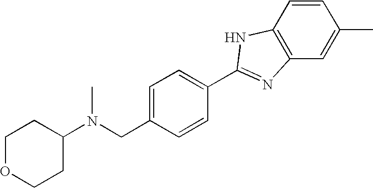

### Step A:

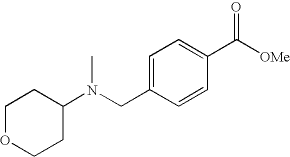

To a solution of 4-methoxycarbonylbenzyl amine HCl salt (3.025 g, 15 mmol) in dichloroethane (34 mL) was added tetrahydro-4H-pyran-4-one (1.4 mL, 15.15 mmol), triethyl amine (2.1 mL, 15.15 mmol), and sodium triacetoxyboron hydride (4.45 g, 21 mmol) at 0° C., and the mixture was warmed to room temperature and stirred for 2 hours at room temperature before re-cooled to 0° C. An aqueous formaldehyde solution (1.23 mL, 37%, 16.5 mmol) and another portion of sodium triacetoxyboronhydride (4.45 g, 21 mmol) were added at 0° C., and the mixture was warmed to room temperature and stirred for 16 hours at room temperature. The volatiles were removed and the mixture was neutralized with an aqueous saturated sodium bicarbonate solution, and extracted with ethyl acetate. The combined extracts were washed with water, brine, dried over magnesium sulfate and concentrated to offer the desired methyl ester as colorless oil (4.0 g). ESI-MS calc. for C15H21NO3: 263; Found: 264 (M+H).

### Step B:

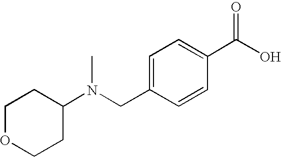

To a solution of the methyl ester (3.5 g, 13.29 mmol) from Step A immediately above in THF/methanol (50/20 mL) was added a 2N aqueous sodium hydroxide solution (26.5 mL, 53 mmol) at room temperature, and the mixture was stirred for 5 hours at room temperature. The pH of the mixture was adjusted to ˜7 with 1N HCl, and the mixture was concentrated down before taken up by chloform/2-propanol (85/15). The organic phase was dried over magnesium sulfate and concentrated to offer the desired acid as white solids (3.05 g). ESI-MS calc. for C14H19NO3: 249; Found: 250 (M+H).

### Step C:

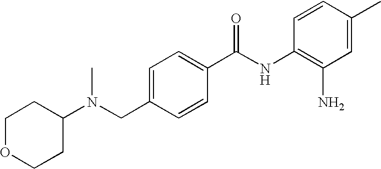

The acid from Step B immediately above (50 mg, 0.2 mmol) was combined in DCM (2 mL) with 4-methylphenylene1,2-diamine (32 mg, 0.26 mmol), EDCI (50 mg, 0.13 mmol), 4-dimethylaminopyridine (2.5 mg, 0.02 mmol), and diisopropyl ethyl amine (0.105 mL, 0.6 mmol). The resulting mixture was stirred for 24 h at room temperature. The reaction mixture was concentrated and purified by preparative TLC (silica, DCM/methanol=11/1) to afford 33 mg of the title product as a thick oil.

ESI-MS calc. for C21H27N3O2: 353; Found: 354 (M+H).

### Step D:

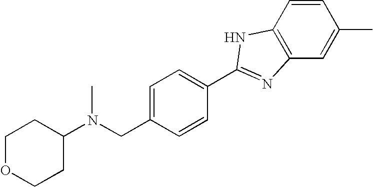

The amide from Step C immediately above (25 mg, 0.07 mmol) was dissolved in glacial acetic acid (1 mL) and the solution was heated to 70° C. for 16 h. Volatiles were removed and the residue was purified by preparative TLC (silica, DCM/methanol=11/1) to afford 24 mg of the title product as off-white solids.

ESI-MS calc. for C21H25N3O: 335; Found: 336 (M+H).

### Example 79

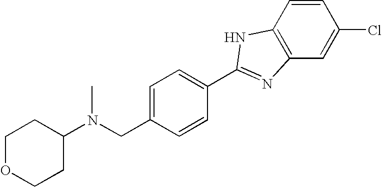

### Step A:

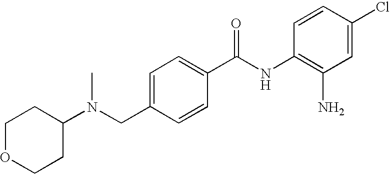

The acid from Step B of Example 1 (50 mg, 0.2 mmol) was combined in DCM (2 mL) with 4-chlorophenylene1,2-diamine (37 mg, 0.26 mmol), EDCI (50 mg, 0.13 mmol), 4-dimethylaminopyridine (2.5 mg, 0.02 mmol), and diisopropyl ethyl amine (0.105 mL, 0.6 mmol). The resulting mixture was stirred for 24 h at room temperature. The reaction mixture was concentrated and purified by preparative TLC (silica, DCM/methanol=11/1) to afford 36 mg of the title product as a thick oil.

ESI-MS calc. for C20H24N3O2Cl: 373; Found: 374 (M+H).

### Step B:

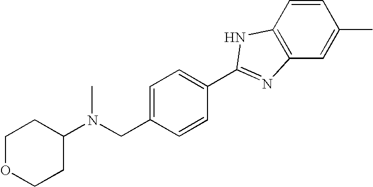

The amide from Step A immediately above (29 mg, 0.078 mmol) was dissolved in glacial acetic acid (1 mL) and the solution was heated to 70° C. for 16 h. Volatiles were removed and the residue was purified by preparative TLC (silica, DCM/methanol=11/1) to afford 27.5 mg of the title product as off-white solids.

ESI-MS calc. for C20H22N30Cl: 355; Found: 356 (M+H).

### Example 80

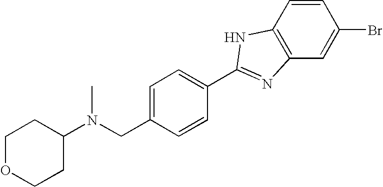

### Step A:

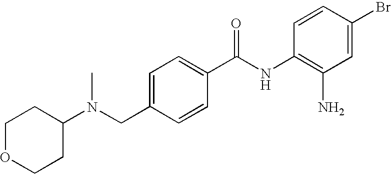

The acid from Step B of Example 1 (50 mg, 0.2 mmol) was combined in DCM (2 mL) with 4-bromophenylene1,2-diamine (49 mg, 0.26 mmol), EDCI (50 mg, 0.13 mmol), 4-dimethylaminopyridine (2.5 mg, 0.02 mmol), and diisopropyl ethyl amine (0.105 mL, 0.6 mmol). The resulting mixture was stirred for 24 h at room temperature. The reaction mixture was concentrated and purified by preparative TLC (silica, DCM/methanol=11/1) to afford 36 mg of the title product as a thick oil.

ESI-MS calc. for C20H24N3O2Br: 417; Found: 418 (M+H).

### Step B:

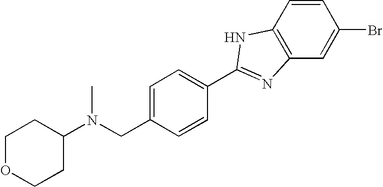

The amide from Step A immediately above (26 mg, 0.062 mmol) was dissolved in glacial acetic acid (1 mL) and the solution was heated to 70° C. for 16 h. Volatiles were removed and the residue was purified by preparative TLC (silica, DCM/methanol=11/1) to afford 25 mg of the title product as off-white solids.

ESI-MS calc. for C20H22N3OBr: 399; Found: 400 (M+H).

### Example 81

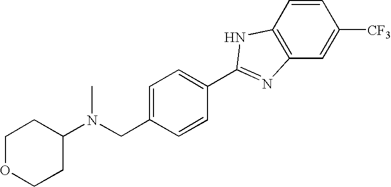

### Step A:

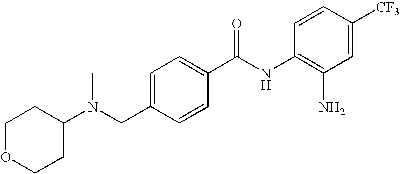

The acid from Step B of Example 1 (50 mg, 0.2 mmol) was combined in DCM (2 mL) with 4-trifluoromethylphenylene1,2-diamine (46 mg, 0.26 mmol), EDCI (50 mg, 0.13 mmol), 4-dimethylaminopyridine (2.5 mg, 0.02 mmol), and diisopropyl ethyl amine (0.105 mL, 0.6 mmol). The resulting mixture was stirred for 24 h at room temperature. The reaction mixture was concentrated and purified by preparative TLC (silica, DCM/methanol=11/1) to afford 36 mg of the title product as off-white solids.

ESI-MS calc. for C21H24N3F3O2: 407; Found: 408 (M+H).

### Step B:

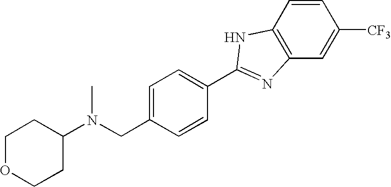

The amide from Step A immediately above (28 mg, 0.069 mmol) was dissolved in glacial acetic acid (1 mL) and the solution was heated to 70° C. for 16 h. Volatiles were removed and the residue was purified by preparative TLC (silica, DCM/methanol=11/1) to afford 26 mg of the title product as off-white solids.

ESI-MS calc. for C21H22N3F3O: 389; Found: 390 (M+H).

### Example 82

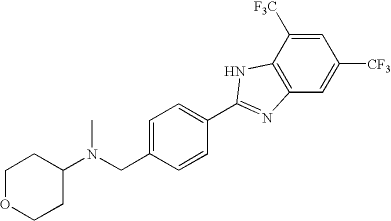

### Step A:

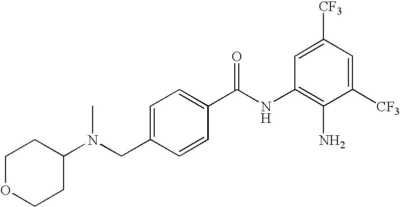

The acid from Step B of Example 1 (50 mg, 0.2 mmol) was combined in DCM (2 mL) with 3,5-bistrifluoromethylphenylene1,2-diamine (63.5 mg, 0.26 mmol), EDU (50 mg, 0.13 mmol), 4-dimethylaminopyridine (2.5 mg, 0.02 mmol), and diisopropyl ethyl amine (0.105 mL, 0.6 mmol). The resulting mixture was stirred for 24 h at room temperature. The reaction mixture was concentrated and purified by preparative TLC (silica, DCM/methanol=11/1) to afford 24 mg of the title product as white solids.

ESI-MS calc. for C22H23N3F6O2: 475; Found: 476 (M+H).

### Step B:

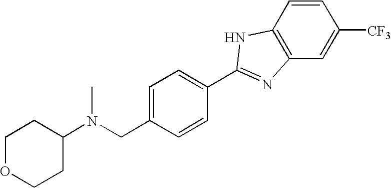

The amide from Step A immediately above (18 mg, 0.038 mmol) was dissolved in glacial acetic acid (1 mL) and the solution was heated to 70° C. for 16 h. Volatiles were removed and the residue was purified by preparative TLC (silica, DCM/methanol=11/1) to afford 16 mg of the title product as white solids.

ESI-MS calc. for C22H21N3F6O: 457; Found: 458 (M+H).

### Example 83

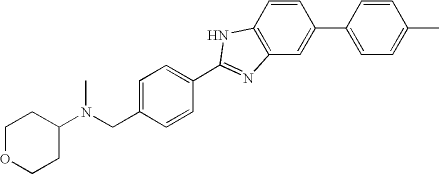

The bromide from Step B of Example 13 (21 mg, 0.052 mmol) was combined in toluene/ethanol/water (0.9/0.3/0.3 mL) with p-tolyboronic acid (8.6 mg, 0.063 mmol), potassium carbonate (25 mg, 0.182 mmol), and tetrakistriphenylphosphine palladium (0) (11.6 mg, 0.01 mmol) under nitrogen. The resulting mixture was refluxed for 3.5 h and cooled down to room temperature. Water was added and the reaction mixture was extracted with ethyl acetate (×3). The combined extracts were dried over magnesium sulfate and concentrated. The residue was purified by preparative TLC (silica, DCM/methanol=10/1) to afford 7 mg of the title product as off-white solids.

ESI-MS calc. for C27H29N3O: 411; Found: 412 (M+H).

### Example 84

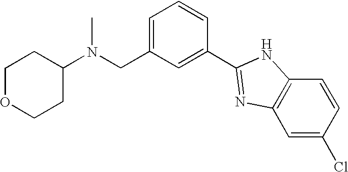

### Step A:

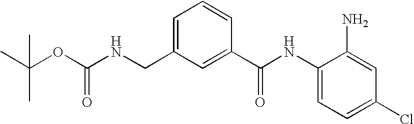

3-(tert-Butoxycarbonylaminomethyl)benzoic acid (151 mg, 0.6 mmol) was combined in DCM (2 mL) with 4-chlorophenylene1,2-diamine (111.2 mg, 0.78 mmol), EDCI (149.5 mg, 0.78 mmol), and 4-dimethylaminopyridine (7.3 mg, 0.06 mmol). The resulting mixture was stirred for 16 h at room temperature. The reaction mixture was concentrated and purified by preparative TLC (silica, DCM/ethyl acetate=5/1) to afford 157 mg of the title product as white solids.

ESI-MS calc. for C19H22ClN3O3: 375; Found: 398 (M+Na).

### Step B:

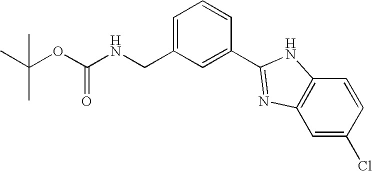

The amide from Step A immediately above (157 mg, 0.418 mmol) was dissolved in glacial acetic acid (3 mL) and the solution was heated to 70° C. for 6 h. Volatiles were removed and the residue was purified by preparative TLC (silica, DCM/ethyl acetate=20/1) to afford 156 mg of the title product as light yellow solids.

ESI-MS calc. for C19H20ClN3O2: 357; Found: 358 (M+H).

### Step C:

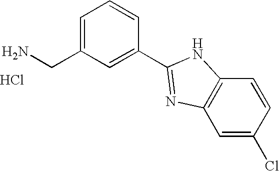

A 4 N solution of HCl in dioxane (1 mL) was added to the benzimidazole from Step B immediately above (78 mg, 0.22 mmol), and the mixture was stirred for 4 h at room temperature. All volatiles were removed to afford the title product.

ESI-MS calc. for C14H12ClN3: 257; Found: 258 (M+H).

### Step D:

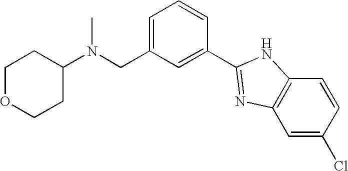

To a solution of amine HCl salt obtained in Step C immediately above (78 mg, 0.022 mmol) in dichloroethane (2 mL) was added tetrahydro-4H-pyran-4-one (0.02 mL, 0.022 mmol), triethyl amine (0.067 mL, 0.048 mmol), and sodium triacetoxyboron hydride (65 mg, 0.305 mmol) at 0° C., and the mixture was warmed to room temperature and stirred for 15 hours at room temperature before re-cooled to 0° C. An aqueous formaldehyde solution (0.018 mL, 37%, 0.24 mmol) and another portion of sodium triacetoxyboronhydride (65 mg, 0.035 mmol) were added at 0° C., and the mixture was warmed to room temperature and stirred for 5 hours at room temperature. Volatiles were removed and the residue was purified by preparative TLC (silica, DCM/methanol=10/1) to afford 68 mg of the title product as white solids.

ESI-MS calc. for C20H22ClN3O: 355; Found: 356 (M+H).

### Example 85

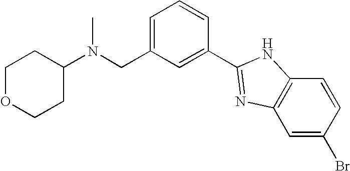

### Step A:

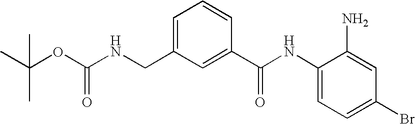

3-(tert-Butoxycarbonylaminomethyl)benzoic acid (151 mg, 0.6 mmol) was combined in DCM (2 mL) with 4-bromophenylene1,2-diamine (146 mg, 0.78 mmol), EDCI (149.5 mg, 0.78 mmol), and 4-dimethylaminopyridine (7.3 mg, 0.06 mmol). The resulting mixture was stirred for 16 h at room temperature. The reaction mixture was concentrated and purified by preparative TLC (silica, DCM/ethyl acetate=5/1) to afford 221 mg of the title product as white solids.

ESI-MS calc. for C19H22BrN3O3: 419; Found: 442 (M+Na).

### Step B:

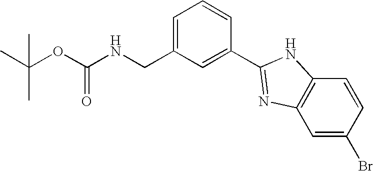

The amide from Step A immediately above (221 mg, 0.526 mmol) was dissolved in glacial acetic acid (3 mL) and the solution was heated to 70° C. for 6 h. Volatiles were removed and the residue was purified by preparative TLC (silica, DCM/ethyl acetate=20/1) to afford 188 mg of the title product as light yellow solids.

ESI-MS calc. for C19H20BrN3O2: 401; Found: 402 (M+H).

### Step C:

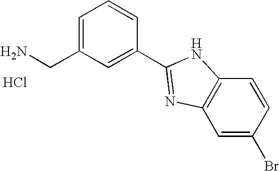

A 4 N solution of HCl in dioxane (1 mL) was added to the benzimidazole from Step B immediately above (97 mg, 0.24 mmol), and the mixture was stirred for 4 h at room temperature. All volatiles were removed to afford the title product.

ESI-MS calc. for C14H12BrN3: 301; Found: 302 (M+H).

### Step D:

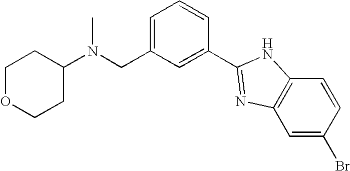

To a solution of amine HCl salt obtained in Step C immediately above (97 mg, 0.024 mmol) in dichloroethane (2 mL) was added tetrahydro-4H-pyran-4-one (0.022 mL, 0.024 mmol), triethyl amine (0.074 mL, 0.053 mmol), and sodium triacetoxyboron hydride (71 mg, 0.336 mmol) at 0° C., and the mixture was warmed to room temperature and stirred for 15 hours at room temperature before re-cooled to 0° C. An aqueous formaldehyde solution (0.02 mL, 37%, 0.26 mmol) and another portion of sodium triacetoxyboronhydride (71 mg, 0.336 mmol) were added at 0° C., and the mixture was warmed to room temperature and stirred for 5 hours at room temperature. Volatiles were removed and the residue was purified by preparative TLC (silica, DCM/methanol=10/1) to afford 62 mg of the title product as white solids.

ESI-MS calc. for C20H22BrN3O: 399; Found: 400 (M+H).

### Example 86

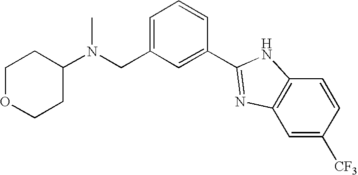

### Step A:

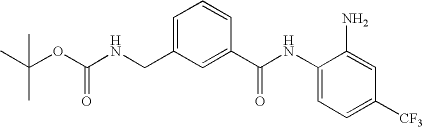

3-(tert-Butoxycarbonylaminomethyl)benzoic acid (151 mg, 0.6 mmol) was combined in DCM (2 mL) with 4-trichloromethylphenylene1,2-diamine (137 mg, 0.78 mmol), EDCI (149.5 mg, 0.78 mmol), and 4-dimethylaminopyridine (7.3 mg, 0.06 mmol). The resulting mixture was stirred for 16 h at room temperature. The reaction mixture was concentrated and purified by preparative TLC (silica, DCM/ethyl acetate=5/1) to afford 230 mg of the title product as white solids.

ESI-MS calc. for C20H22F3N3O3: 409; Found: 432 (M+Na).

### Step B:

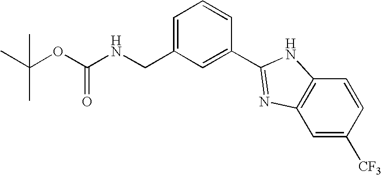

The amide from Step A immediately above (230 mg, 0.562 mmol) was dissolved in glacial acetic acid (3 mL) and the solution was heated to 70° C. for 6 h. Volatiles were removed and the residue was purified by preparative TLC (silica, DCM/ethyl acetate=20/1) to afford 213 mg of the title product as light yellow solids.

ESI-MS calc. for C20H20F3N3O2: 391; Found: 392 (M+H).

### Step C:

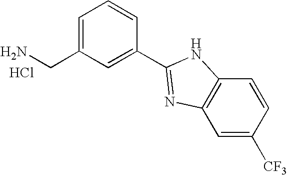

A 4 N solution of HCl in dioxane (1 mL) was added to the benzimidazole from Step B immediately above (108 mg, 0.22 mmol), and the mixture was stirred for 4 h at room temperature. All volatiles were removed to afford the title product.

ESI-MS calc. for C15H12F3N3: 291; Found: 292 (M+H).

### Step D:

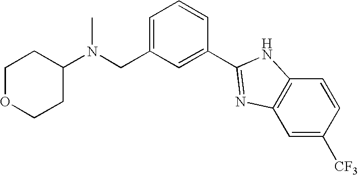

To a solution of amine HCl salt obtained in Step C immediately above (108 mg, 0.026 mmol) in dichloroethane (2 mL) was added tetrahydro-4H-pyran-4-one (0.024 mL, 0.026 mmol), triethyl amine (0.08 mL, 0.057 mmol), and sodium triacetoxyboron hydride (77 mg, 0.364 mmol) at 0° C., and the mixture was warmed to room temperature and stirred for 15 hours at room temperature before re-cooled to 0° C. An aqueous formaldehyde solution (0.021 mL, 37%, 0.286 mmol) and another portion of sodium triacetoxyboronhydride (77 mg, 0.0364 mmol) were added at 0° C., and the mixture was warmed to room temperature and stirred for 5 hours at room temperature. Volatiles were removed and the residue was purified by preparative TLC (silica, DCM/methanol=10/1) to afford 77 mg of the title product as white solids.

ESI-MS calc. for C21H22F3N3O: 389; Found: 390 (M+H).

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A compound of formula I or formula II: 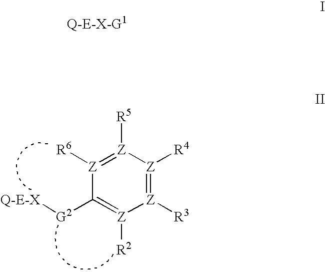
wherein:
Q is: 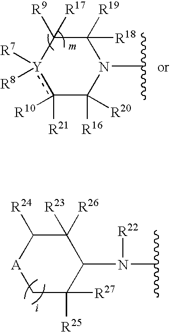
A is selected from: —O—, —NR¹²—, —S—, —SO—, —SO₂—, —CR¹²R¹²—, —NSO₂R¹⁴—, —NCOR¹³—, —CR¹²COR¹¹—, —CR¹²OCOR¹³— and —CO—;
E is: 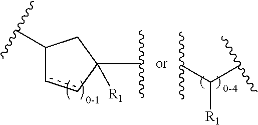
G¹ is selected from: —N(R³¹)—CO—N(R³⁰)(R²⁹), —N(R³¹)—SO₂R³², —N(R³¹)—COR³², —CON(R²⁹)(R³⁰), —C₁₋₆alkyl unsubstituted or substituted with 1-6 fluoro, and —C₃₋₆cycloalkyl unsubstituted or substituted with 1-6 fluoro,
where R²⁹ and R³⁰ are independently selected from: hydrogen, C₁₋₆alkyl, C₁₋₆alkyl substituted with 1-6 fluoro, C₁₋₆cycloalkyl, aryl, aryl-C₁₋₆alkyl, heterocycle and heterocycle-C₁₋₆alkyl, or R²⁹ and R³⁰ join to form a C₃₋₆ membered ring;
where R³¹ and R³² are independently selected from: hydrogen, C₁₋₆alkyl, C₁₋₆cycloalkyl, C₁₋₆alkyl substituted with 1-6 fluoro, aryl and heterocycle, or R³¹ and R³² join to form a C₃₋₆membered ring;
G² is selected from: a single bond, —(CR¹¹R¹¹)₁₄—, —N(R¹²)SO₂—, —N(R¹²)SO₂N(R¹²)—, —N(R¹²)CO—, —C(R¹¹)(R¹¹)CO—, —C(R¹¹)(R¹¹)OCO—, —CO—, —C(R¹¹)(R¹¹)SO₂—, —OCO—, —SO₂—, or G² is C R¹¹ or N and is joined to R² faulting a fused carbocyclic or heterocyclic ring;
X is a 5-7 membered saturated, partially unsaturated or unsaturated carbocyclic or heterocyclic ring, wherein:
when said ring is heterocyclic it contains 1-4 heteroatoms independently selected from O, N and S,
said ring is unsubstituted or substituted with 1-4 R²⁸,
R²⁸ is independently selected from: halo, hydroxy, —O—C₁₋₃alkyl unsubstituted or substituted with 1-6 fluoro, C₁₋₃alkyl unsubstituted or substituted with 1-6 fluoro, —O—C₃₋₅cycloalkyl unsubstituted or substituted with 1-6 fluoro, —COR11, —SO2R14, —NR¹²COR¹³, —NR¹²SO₂R¹⁴, -phenyl unsubstituted or substituted with 1-3 fluoro or trifluoromethyl, and —CN, and
said ring is optionally bonded to R⁶ to form a fused or spiro ring system;
Y is C, N, O, S or SO₂;
Z is independently selected from C and N, where no more than two of Z are N;
R¹ is selected from: hydrogen, —SO₂R¹⁴, —C₀₋₃alkyl-S(O)R¹⁴, —SO₂NR¹²R¹², —C₁₋₆alkyl, —C₀₋₆alkyl-O—C₁₋₆alkyl, —C₀₋₆alkyl-S—C₁₋₆alkyl, —(C₀₋₆alkyl)-(C₃₋₇cycloalkyl)-(C₀₋₆alkyl), hydroxy, heterocycle, —CN, —NR¹²R¹², —NR¹²COR¹³, —NR¹²SO₂R¹⁴, —COR¹¹, —CONR¹²R¹², and phenyl,
wherein said alkyl and the cycloalkyl are unsubstituted or substituted with 1-7 substituents where the substituents are independently selected from: halo, hydroxy, —O—C₁₋₃alkyl, trifluoromethyl, C₁₋₃alkyl, —O—C₃₋₅cycloalkyl, —COR¹¹, —SO₂R¹⁴, —NHCOCH₃, —NHSO₂CH₃, -heterocycle, ═O and —CN, and
wherein said phenyl and heterocycle are unsubstituted or substituted with 1-3 substituents independently selected from halo, hydroxy, C₁₋₃alkyl, C₁₋₃alkoxy and trifluoromethyl;
R³, R⁴, and R⁵ are independently selected from B¹ when Z is C, and are independently selected from B² when Z is N;
R² is independently selected from B′ when Z is C, and is independently selected from B² when Z is N, or R² is a link to G² wherein said link is a bond or is a chain 1-4 atoms in length where said atoms are independantly selected from O, N, C and S and where said atoms are independantly joined by single or double bonds, said link forming a fused carbocyclic or heterocyclic ring;
R⁶ is independently selected from B′ when Z is C, and is independently selected from B² when Z is N, or R⁶ is a link to any atom on X, wherein said link is a bond or is a chain 1-3 atoms in length where said atoms are independantly selected from O, N, C and S and where said atoms are independantly joined by single or double bonds, said link forming a fused carbocyclic or heterocyclic ring;
B¹ is selected from: C₁₋₆alkyl unsubstituted or substituted with 1-6 fluoro, hydroxyl, or both, —O—C₁₋₆alkyl unsubstituted or substituted with 1-6 fluoro, —CO—C₁₋₆alkyl unsubstituted or substituted with 1-6 fluoro, —S—C₁₋₆alkyl unsubstituted or substituted with 1-6 fluoro, -pyridyl unsubstituted or substituted with one or more substituents selected from the group consisting of: halo, trifluoromethyl, C₁₋₄alkyl and COR¹¹, fluoro, chloro, bromo, —C₄₋₆cycloalkyl, —O—C₄₋₆cycloalkyl, phenyl unsubstituted or substituted with one or more substituents selected from halo, trifluoromethyl, C₁₋₄alkyl and COR¹¹, —O-phenyl unsubstituted or substituted with one or more substituents selected from halo, trifluoromethyl, C₁₋₄alkyl and COR¹¹, —C₃₋₆cycloalkyl unsubstituted or substituted with 1-6 fluoro, —O—C₃₋₆cycloalkyl unsubstituted or substituted with 1-6 fluoro, -heterocycle, —CN, —COR¹¹ and hydrogen;
B² is absent or is O, forming an N-oxide;
R⁷ is selected from: hydrogen, (C₀₋₆alkyl)-phenyl, (C₀₋₆alkyl)-heterocycle, (C₀₋₆alkyl)-C₃₋₇cycloalkyl, (C₀₋₆alkyl)-COR¹¹, (C₀₋₆alkyl)-(alkene)-COR¹¹, (C₀₋₆alkyl)-SO₃H, (C₀₋₆alkyl)-W—C₀₋₄alkyl, (C₀₋₆alkyl)-CONR¹²-pheny and (C₀₋₆alkyl)-CONR¹⁵—V—COR¹¹ when Y is N or C, or R⁷ is absent when Y is O, S or SO₂, where
V is C₁₋₆alkyl or phenyl,
W is a single bond, —O—, —S—, —SO—, —SO₂—, —CO—, —CO₂—, —CONR¹²— or —NR¹²—,
R¹⁵ is hydrogen or C₁₋₄alkyl, or R¹⁵ is joined via a 1-5 carbon chain linked to one of the carbons of V, forming a ring,
said C₀₋₆alkyl is unsubstituted or substituted with 1-5 substituents independently selected from halo, hydroxy, —C₀₋₆alkyl, —O—C₁₋₃alkyl, trifluoromethyl and —C₀₋₂alkyl-phenyl,
said phenyl, heterocycle, cycloalkyl and C₀₋₄alkyl are unsubstituted or substituted with 1-5 substituents independently selected from halo, trifluoromethyl, hydroxy, C₁₋₆alkyl, —O—C₁₋₃alkyl, —C₀₋₃—COR¹¹, —CN, —NR¹²R¹², —CONR¹²R¹² and —C₀₋₃-heterocycle, or said phenyl or heterocycle may be fused to another heterocycle where said another heterocycle is unsubstituted or substituted with 1-2 substituents independently selected from hydroxy, halo, —COR¹¹, and —C₁₋₄alkyl, and
said alkene is unsubstituted or substituted with 1-3 substituents independently selected from halo, trifluoromethyl, C₁₋₃alkyl, phenyl and heterocycle;
R⁸ is selected from hydrogen, hydroxy, C₁₋₆alkyl, C₁₋₆alkyl-hydroxy, —O—C₁₋₃alkyl, —COR¹¹, —CONR¹²R¹² and —CN when Y is N or C, or R⁸ is absent when Y is O, S, SO₂ or N or when a double bond joins the carbons to which R⁷ and R¹⁰ are attached;
or R⁷ and R⁸ are joined to form a ring selected from: 1H-indene, 2,3-dihydro-1H-indene, 2,3-dihydro-benzofuran, 1,3-dihydro-isobenzofuran, 2,3-dihydro-benzothiofuran, 1,3-dihydro-isobenzothiofuran, 6H-cyclopenta[d]isoxazol-3-ol, cyclopentane and cyclohexane,
where said ring is unsubstituted or substituted with 1-5 substituents independently selected from: halo, trifluoromethyl, hydroxy, C₁₋₃alkyl, —O—C₁₋₃alkyl, —C₀₋₃—COR¹¹, —CN, —NR¹²R¹², —CONR¹²R¹² and —C₀₋₃-heterocycle;
R⁹ and R¹⁰ are independently selected from: hydrogen, hydroxy, C₁₋₆alkyl, C₁₋₆alkyl-COR¹¹, C₁₋₆alkyl-hydroxy, —O—C₁₋₃alkyl, halo and ═O;
or R⁷ and R⁹, or R⁸ and R¹⁰, together form a ring which is phenyl or heterocycle, wherein said ring is unsubstituted or substituted with 1-7 substituents independently selected from halo, trifluoromethyl, hydroxy, C₁₋₃alkyl, —O—C₁₋₃alkyl, —COR¹¹, —CN, —NR¹²R¹² and —CONR¹²R¹²;
R¹¹ is independently selected from: hydroxy, hydrogen, C₁₋₆ alkyl, —O—C₁₋₆alkyl, benzyl, phenyl and C₃₋₆cycloalkyl, where said alkyl, phenyl, benzyl and cycloalkyl are unsubstituted or substituted with 1-3 substituents independently selected from halo, hydroxy, C₁₋₃alkyl, C₁₋₃alkoxy, —CO₂H, —CO₂—C₁₋₆ alkyl and trifluoromethyl;
R¹² is selected from: hydrogen, C₁₋₆ alkyl, benzyl, phenyl and C₃₋₆ cycloalkyl, where said alkyl, phenyl, benzyl, and cycloalkyl are unsubstituted or substituted with 1-3 substituents independently selected from halo, hydroxy, C₁₋₃alkyl, C₁₋₃alkoxy, —CO₂H, —CO₂—C₁₋₆ alkyl, and trifluoromethyl;
R¹³ is selected from: hydrogen, C₁₋₆ alkyl, —O—C₁₋₆alkyl, benzyl, phenyl and C₃₋₆ cycloalkyl, where said alkyl, phenyl, benzyl and cycloalkyl are unsubstituted or substituted with 1-3 substituents independently selected from halo, hydroxy, C₁₋₃alkyl, C₁₋₃alkoxy, —CO₂H, —CO₂—C₁₋₆ alkyl and trifluoromethyl;
R¹⁴ is selected from: hydroxy, C₁₋₆ alkyl, —O—C₁₋₆alkyl, benzyl, phenyl and C₃₋₆ cycloalkyl, where said alkyl, phenyl, benzyl, and cycloalkyl are unsubstituted or substituted with 1-3 substituents independently selected from halo, hydroxy, C₁₋₃alkyl, C₁₋₃alkoxy, —CO₂H, —CO₂—C₁₋₆ alkyl, and trifluoromethyl;
R¹⁶ and R¹⁸ are independently selected from: hydroxy, C₁₋₆alkyl, C₁₋₆alkyl-COR¹¹, C₁₋₆alkyl-hydroxy, —O—C₁₋₃alkyl, halo and hydrogen, where said alkyl is unsubstituted or substituted with 1-6 substituents independantly chosen from fluoro and hydroxyl;
or R¹⁶ and R¹⁸ together are —C₁₋₄alkyl-, —C₀₋₂alkyl-O—C₁₋₃alkyl- or —C₁₋₃alkyl-O—C₀₋₂alkyl-, forming a bridge, where said alkyl groups are unsubstituted or substituted with 1-2 substituents selected from oxy, fluoro, hydroxy, methoxy, methyl and trifluoromethyl;
R¹⁷, R¹⁹, R²⁰ and R²¹ are independently selected from: hydrogen, hydroxy, C₁₋₆alkyl, C₁₋₆alkyl-COR¹¹, C₁₋₆alkyl-hydroxy, —O—C₁₋₃alkyl, trifluoromethyl and halo;
R²² is hydrogen or C₁₋₆alkyl unsubstituted or substituted with 1-3 substituents independently selected from halo, hydroxy, —CO₂H, —CO₂C₁₋₆alkyl and —O—C₁₋₃alkyl;
R²³ is selected from: C₁₋₆alkyl unsubstituted or substituted with 1-6 substituents selected from fluoro, C₁₋₃alkoxy, hydroxyl and —COR¹¹, fluoro, —O—C₁₋₃alkyl unsubstituted or substituted with 1-3 fluoro, C₃₋₆ cycloalkyl, —O—C₃₋₆cycloalkyl, hydroxy, —COR¹¹, —OCOR¹³, and ═O, or R²² and R²³ together are C₂₋₄alkyl or C₀₋₂alkyl-O—C₁₋₃alkyl, forming a 5-7 membered ring;
R²⁴ is selected from: hydrogen, C₁₋₆alkyl unsubstituted or substituted with 1-6 substituents selected from fluoro, C₁₋₃alkoxy, hydroxyl and —COR¹¹, COR¹¹, hydroxyl and —O—C₁₋₆alkyl unsubstituted or substituted with 1-6 substituents selected from fluoro, C₁₋₃alkoxy, hydroxyl and —COR¹¹, or R²³ and R²⁴ together are C₁₋₄alkyl or C₀₋₃alkyl-O—C₀₋₃alkyl, forming a 3-6 membered ring;
R²⁵ is selected from: hydrogen, C₁₋₆alkyl unsubstituted or substituted with 1-6 fluoro, fluoro, —O—C₃₋₆cycloalkyl and —O—C₁₋₃alkyl unsubstituted or substituted with 1-6 fluoro,
or R²³ and R²⁵ together are C₂₋₃alkyl, forming a 5-6 membered ring, where said alkyl is unsubstituted or substituted with 1-3 substituents independently selected from halo, hydroxy, —COR¹¹, C₁₋₃alkyl, and C₁₋₃alkoxy,
or R²³ and R²⁵ together are C₁₋₂alkyl-O—C₁₋₂alkyl, forming a 6-8 membered ring, where said alkyls are unsubstituted or substituted with 1-3 substituents independently selected from halo, hydroxy, —COR¹¹, C₁₋₃alkyl and C₁₋₃alkoxy,
or R²³ and R²⁵ together are —O—C₁₋₂alkyl-O—, forming a 6-7 membered ring, where said alkyl is unsubstituted or substituted with 1-3 substituents independently selected from halo, hydroxy, —COR¹¹, C₁₋₃alkyl and C₁₋₃alkoxy;
R²⁶ is selected from: C₁₋₆alkyl unsubstituted or substituted with 1-6 substituents selected from fluoro, C₁₋₃alkoxy, hydroxyl and —COR¹¹, fluoro, —O—C₁₋₃alkyl unsubstituted or substituted with 1-3 fluoro, C₃₋₆ cycloalkyl, —O—C₃₋₆cycloalkyl, hydroxyl and —COR¹¹, or R²⁶ is absent if R²³ is connected to the Q ring via double bond,
or R²⁶ and R²³ together form a bridgeselected from —C₂₋₅alkyl-, —O—C₂₋₅alkyl-, —O—C₂₋₅alkyl-O—, and —C₁₋₃alkyl-O—C₁₋₃alkyl-, where said alkyls are unsubstituted or substituted with 1-6 fluoro;
R²⁷ is selected from: hydrogen, C₁₋₆alkyl unsubstituted or substituted with 1-6 fluoro, fluoro, —O—C₃₋₆cycloalkyl, and —O—C₁₋₃alkyl unsubstituted or substituted with 1-6 fluoro;
m, i, and n are independently selected from 0, 1 and 2;
the dashed line represents an optional bond;
and pharmaceutically acceptable salts thereof and individual diastereomers thereof.

2. The compound of claim 1 having the formula Ia: 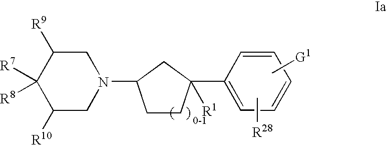
and pharmaceutically acceptable salts and individual diastereomers thereof.

3. The compound of claim 1 having the formula Ib: 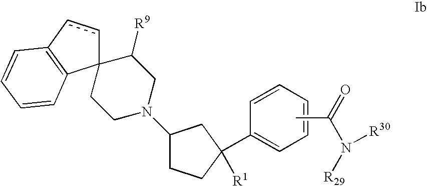
and pharmaceutically acceptable salts and individual diastereomers thereof.

4. The compound of claim 1 having the formula Ic: 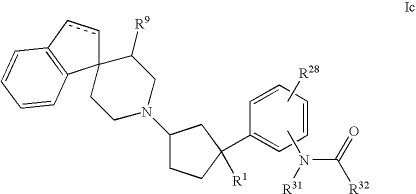
and pharmaceutically acceptable salts and individual diastereomers thereof.

5. The compound of claim 1 having the formula Id: 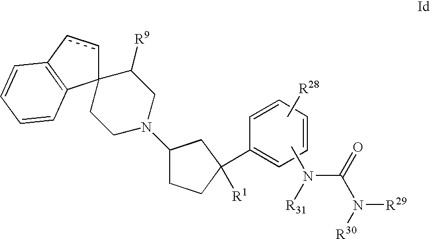
and pharmaceutically acceptable salts and individual diastereomers thereof.

6. The compound of claim 1 having the formula Ie: 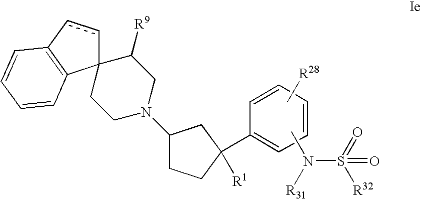
and pharmaceutically acceptable salts and individual diastereomers thereof.

7. The compound of claim 1 having the formula IIa: 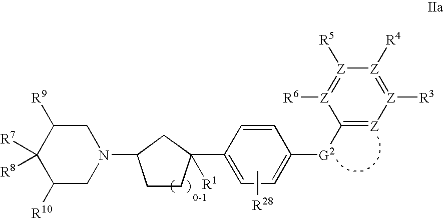
and pharmaceutically acceptable salts and individual diastereomers thereof.

8. The compound of claim 1 having the formula IIb: 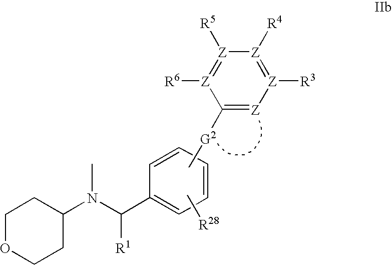
and pharmaceutically acceptable salts and individual diastereomers thereof.

9. The compound of claim 1 having the formula IIc: 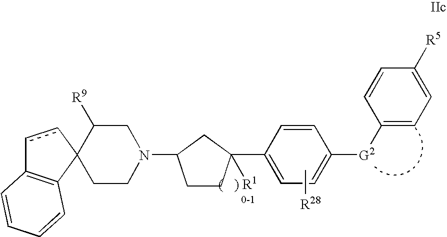
and pharmaceutically acceptable salts and individual diastereomers thereof.

10. The compound of claim 1 having the formula IId: 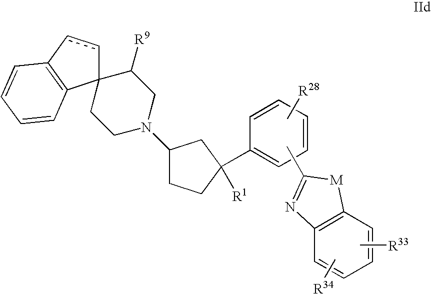
wherein
M is O, S or NR¹²;
R³³ and R³⁴ are independently selected from hydrogen, halo, trifluoromethyl, O—C₁₋₆alkyl and O—C₁₋₆alkyl substituted with 1-6 fluoro, and pharmaceutically acceptable salts and individual diastereomers thereof.

11. The compound of claim 1 having the formula IIe: 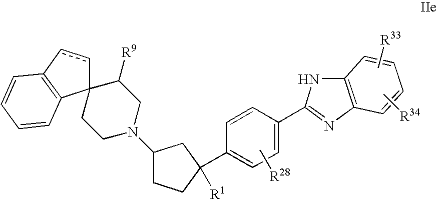
and pharmaceutically acceptable salts and individual diastereomers thereof.

12. The compound of claim 1 wherein R²⁸ is selected from H, F, Cl, Br, Me and CF₃.

13. The compound of claim 1 wherein Y is C.

14. The compound of claim 1 wherein A is O.

15. The compound of claim 1 wherein X is phenyl.

16. The compound of claim 1 wherein R¹ is selected from: hydrogen, —C₁₋₆alkyl unsubstituted or substituted with 1-6 substituents independently selected from halo, hydroxy, —O—C₁₋₃alkyl and trifluoromethyl, —C₀₋₆alkyl-O—C₁₋₆alkyl-unsubstituted or substituted with 1-6 substituents independently selected from halo and trifluoromethyl, —C₀₋₆alkyl-S—C₁₋₆alkyl-unsubstituted or substituted with 1-6 substituents independently selected from halo and trifluoromethyl, —(C₃₋₅cycloalkyl)-(C₀₋₆alkyl) unsubstituted or substituted with 1-7 substituents independently selected from halo, hydroxy, —O—C₁₋₃alkyl and trifluoromethyl.

17. The compound of claim 16 wherein R¹ is selected from hydrogen, C₁₋₆alkyl, C₁₋₆alkyl-hydroxy and C₁₋₆alkyl substituted with 1-6 fluoro, specifically wherein R¹ is selected from hydrogen, methyl, hydroxymethyl and trifluoromethyl.

18. The compound of claim 1 wherein when Z is N, R² is absent.

19. The compound of claim 1 wherein when Z is C, R² is hydrogen or is linked to G².

20. The compound of claim 1 wherein if Z is N, R³ is absent.

21. The compound of claim 1 wherein if Z is C, R³ is hydrogen.

22. The compound of claim 1 wherein if the Z bonded to R⁴ is N, R⁴ is absent.

23. The compound of claim 1 wherein if the Z bonded to R⁴ is C, R⁴ is hydrogen.

24. The compound of claim 1 wherein if the Z bonded to R⁵ is N, R⁵ is absent.

25. The compound of claim 1 wherein if the Z bonded to R⁶ is N, R⁶ is absent.

26. The compound of claim 1 wherein if the Z bonded to R⁶ is C, R⁶ is hydrogen.

27. The compound of claim 1 wherein R⁷ is selected from phenyl, heterocycle, C₃₋₇cycloalkyl, C₁₋₆alkyl, —COR¹¹ and —CONH—V—COR¹¹, where V is C₁₋₆alkyl or phenyl, and where said phenyl, heterocycle, C₃₋₇cycloalkyl and C₁₋₆alkyl is unsubstituted or substituted with 1-5 substituents independently selected from: halo, trifluoromethyl, hydroxy, C₁₋₃alkyl, —O—C₁₋₃alkyl, —COR¹¹, —CN, -heterocycle and —CONR¹²R¹².

28. The compound of claim 1 wherein R⁸ is selected from: hydrogen, hydroxy, —CN and —F.

29. The compound of claim 1 wherein R⁷ and R⁸ are joined together to form a ring selected from: 1H-indene and 2,3-dihydro-1H-indene, where said ring is unsubstituted or substituted with 1-3 substituents independently selected from: halo, hydroxy, C₁₋₃alkyl, —O—C₁₋₃alkyl, —COR¹¹ and -heterocycle.

30. The compound of claim 1 wherein R⁹ and R¹⁰ are independently selected from: hydrogen, hydroxy, —CH₃, —O—CH₃ and ═O.

31. A compound selected from: 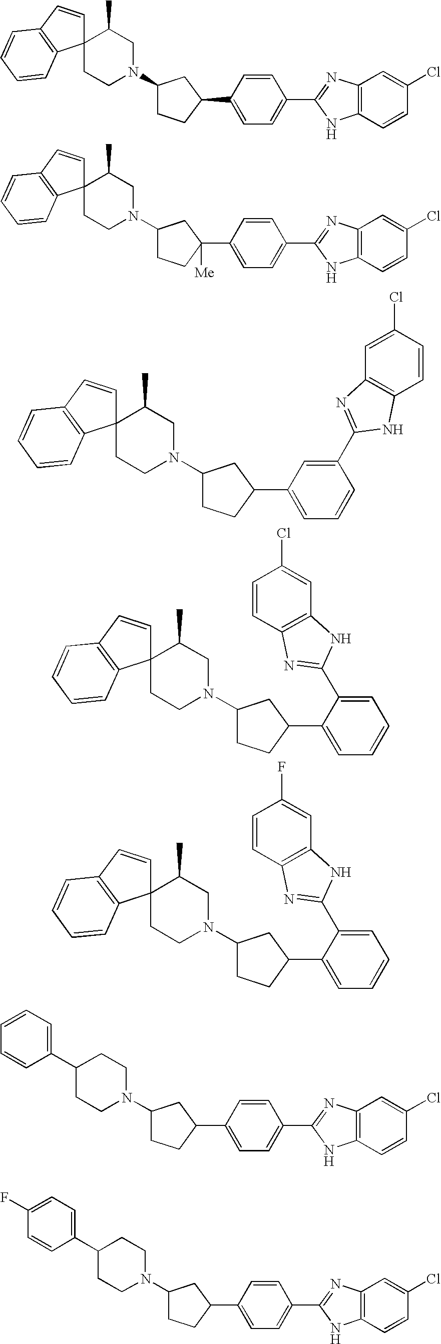 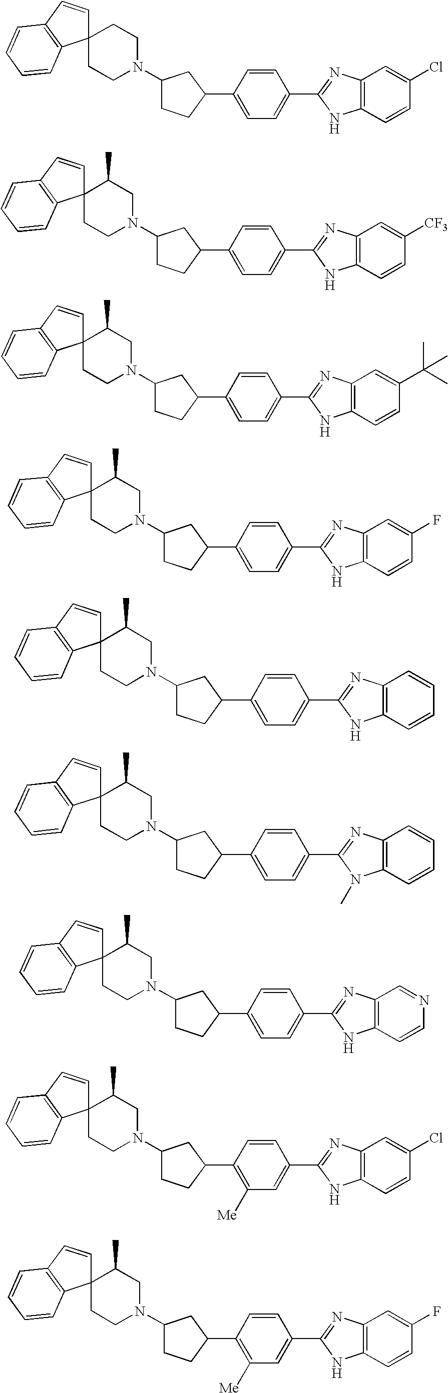 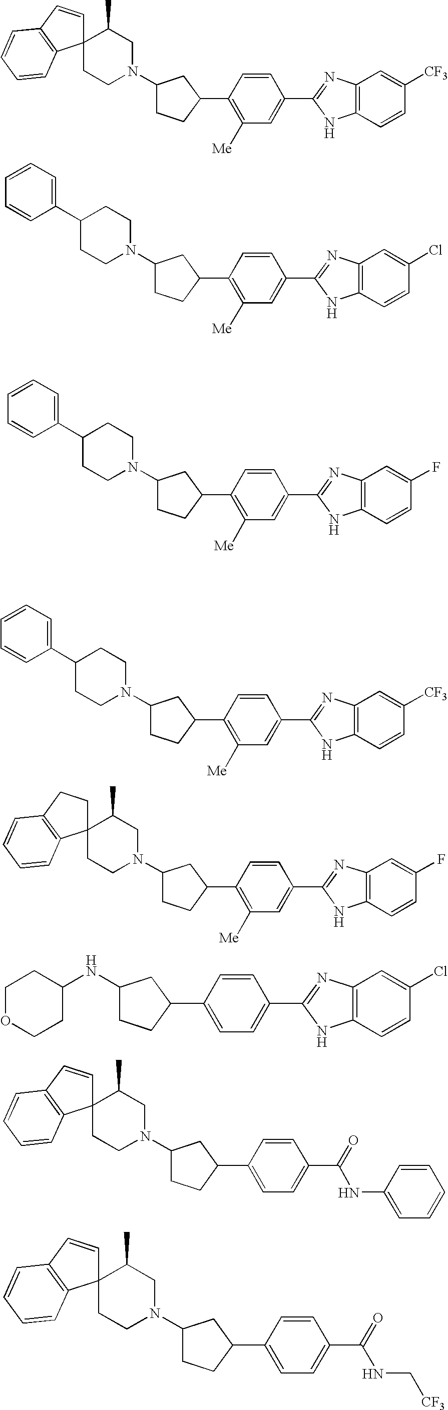 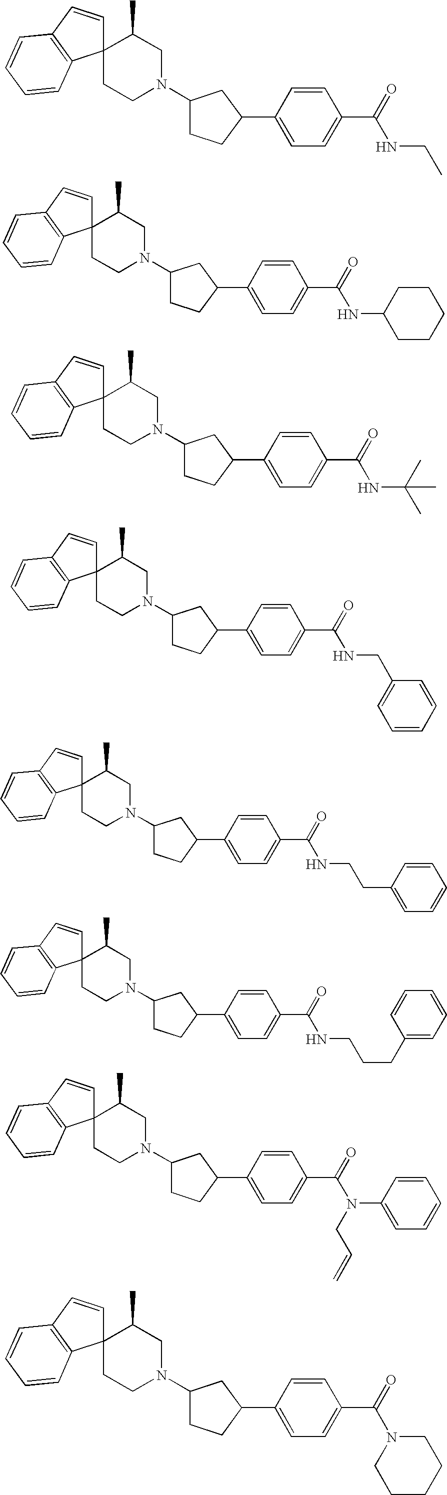 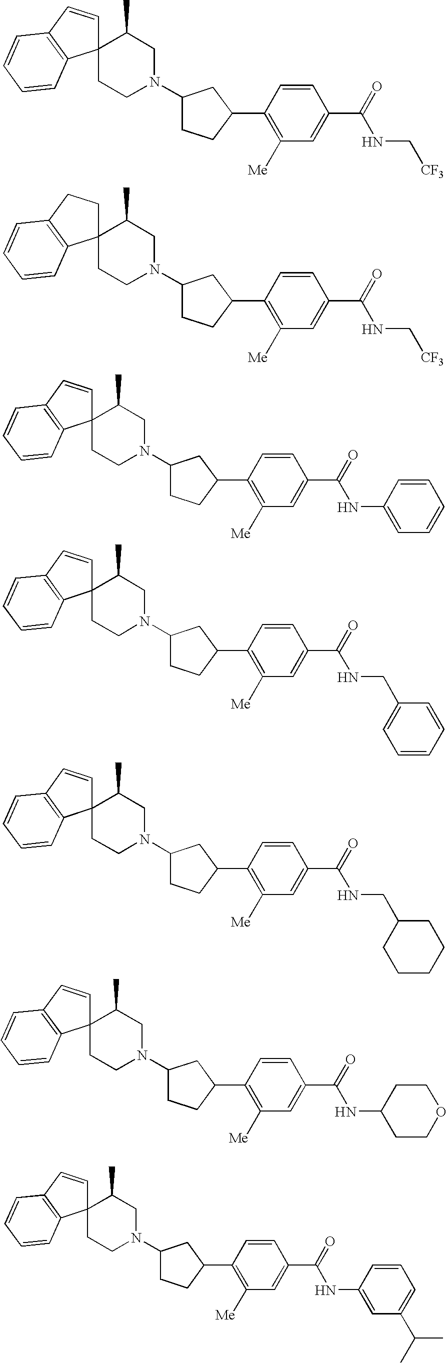 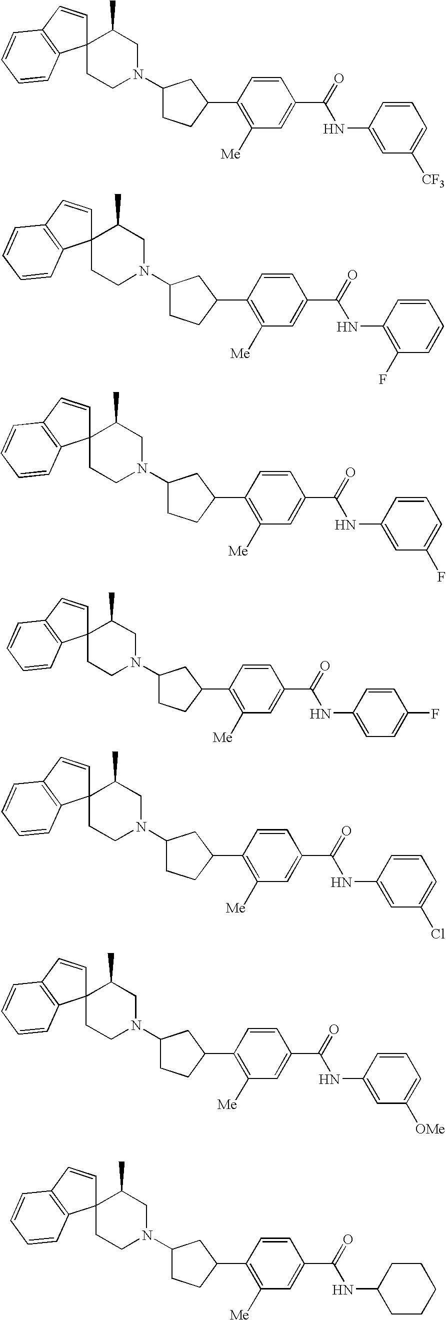 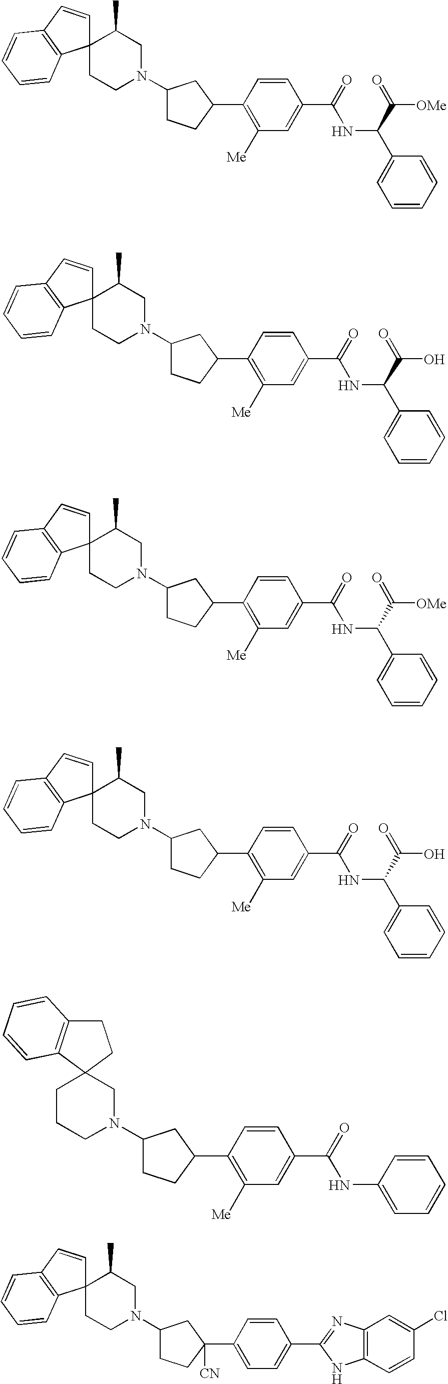 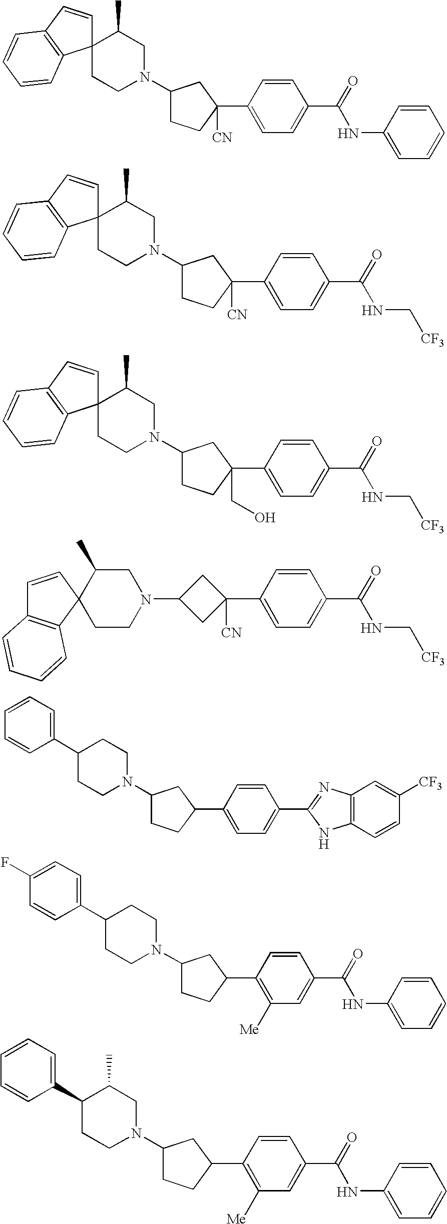 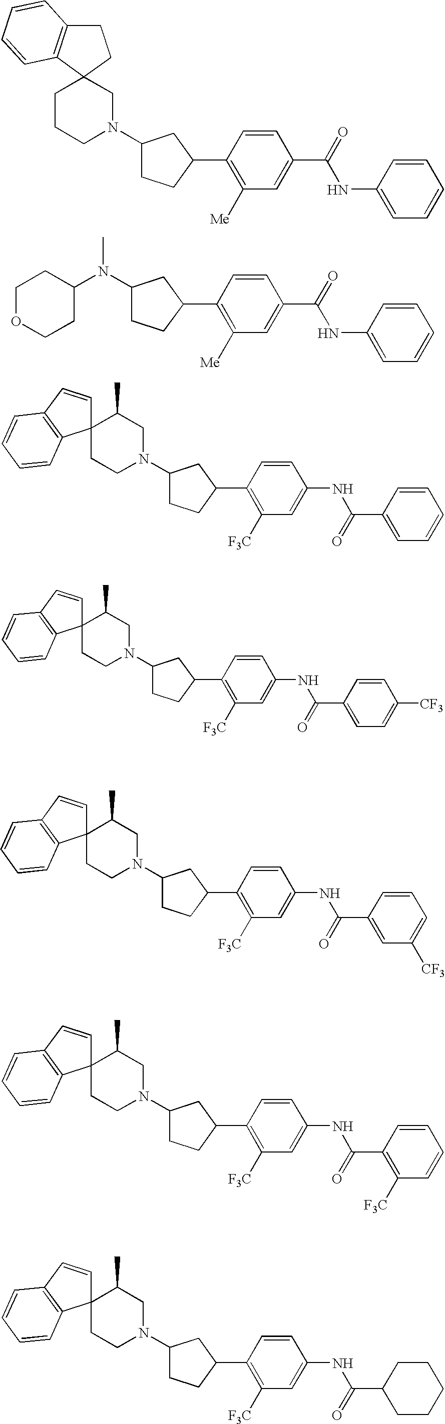 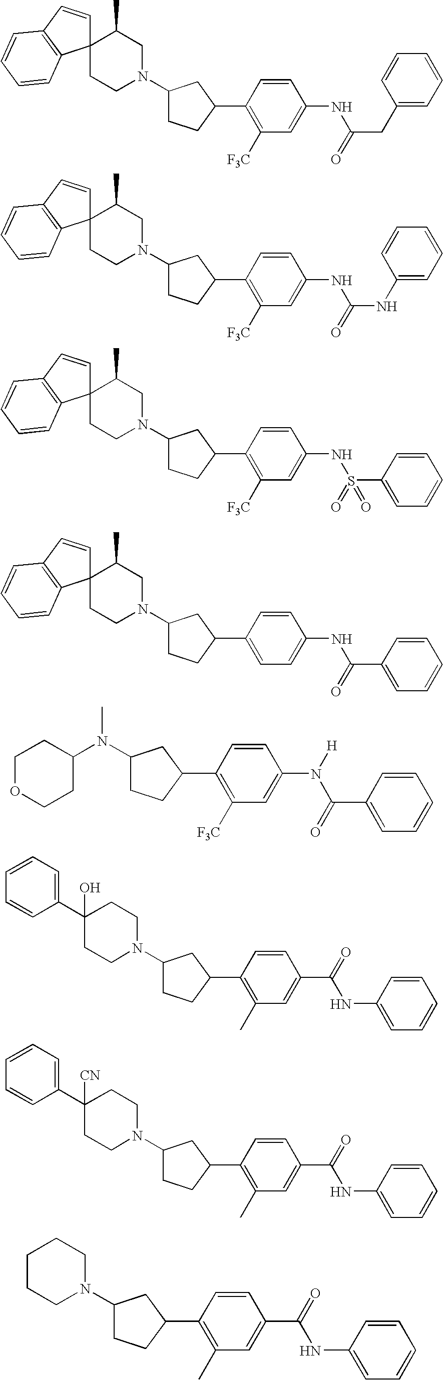 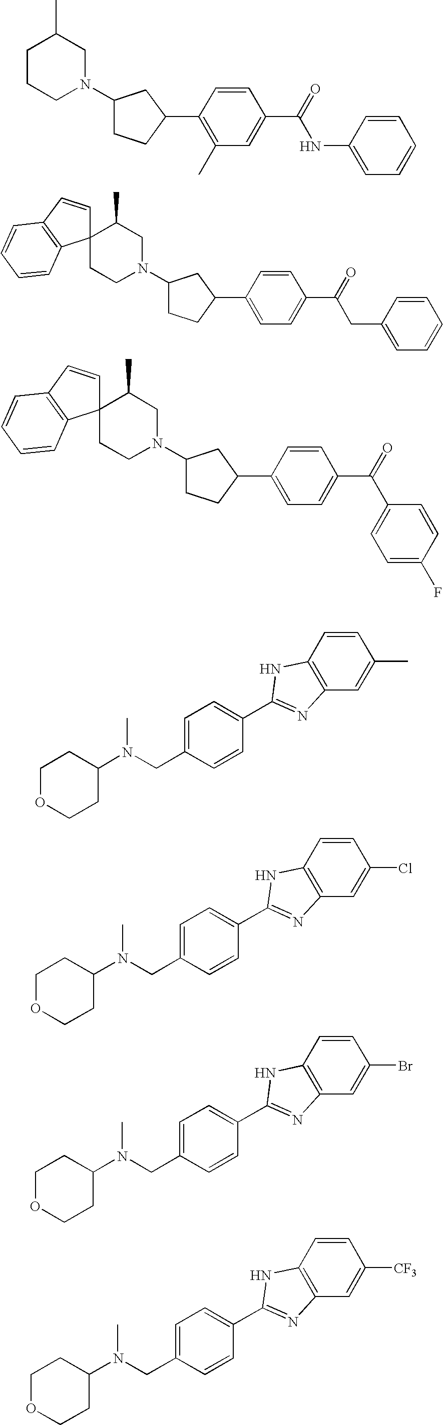 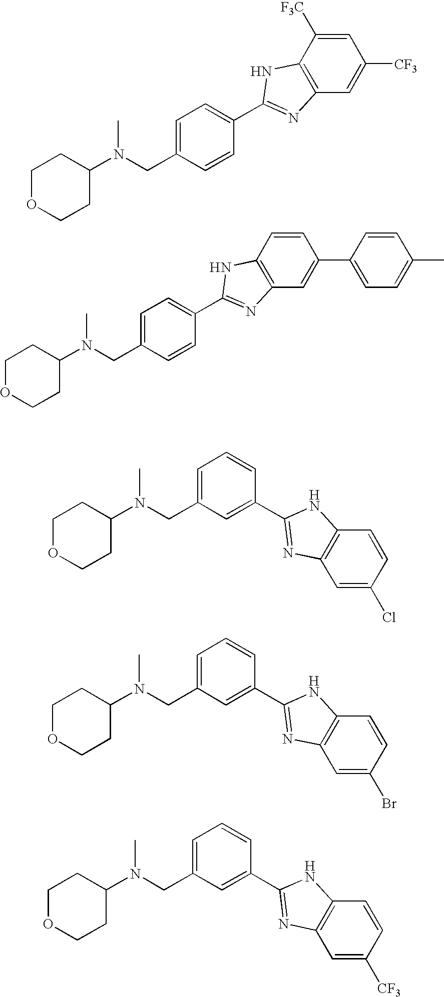
and pharmaceutically acceptable salts thereof and individual diastereomers and enantiomers thereof.

32. A pharmaceutical composition which comprises an inert carrier and the compound of claim 1.

33. The use of the compound of claim 1 for the preparation of a medicament useful in the treatment of an inflammatory and immunoregulatory disorder or disease.

34. The use according to claim 14 wherein said disorder or disease is rheumatoid arthritis.
